(19) United States
(12) Patent Application Publication
(10) Pub. No.: US 2010/0087424 A1
(43) Pub. Date: Apr. 8, 2010

(54) TRICYCLIC NITROGEN CONTAINING HETEROCYCLES AS ANTIBACTERIAL AGENTS

(75) Inventors: Pamela Brown, Hertfordshire (GB); Steven Dabbs, Hertfordshire (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: Glaxo Group Limited

(21) Appl. No.: 12/596,691

(22) PCT Filed: Apr. 17, 2008
(86) PCT No.: PCT/EP08/54655

§ 371 Date: Oct. 20, 2009

(30) Foreign Application Priority Data
Apr. 20, 2007 (GB) 0707708.4

(51) Int. Cl. A61K 31/4745; C07D 471/06; C07D 491/056; C07D 498/16; C07D 513/04; A61P 31/04; A61K 31/5025; A61K 31/5383; A61K 31/542

(52) U.S. Cl. 514/224.2; 546/94; 544/235; 544/101; 544/48; 514/294; 514/248; 514/230.2

(57) ABSTRACT

Tricyclic nitrogen containing compounds and their use as antibacterials.

This invention relates to novel compounds, compositions containing them and their use as antibacterials including use in the treatment of tuberculosis.

WO02/08224, WO02/50061, WO02/56882, WO02/96907, WO2003087098, WO2003010138, WO2003064421, WO2003064431, WO2004002992, WO2004002490, WO2004014361, WO2004041210, WO2004096982, WO2002050036, WO2004058144, WO2004087145, WO06002047, WO06014580, WO06010040, WO06017326, WO06012396, WO06017468, WO06020561, WO06081179, WO06081264, WO06081289, WO06081178, WO06081182, WO01/25227, WO02/40474, WO02/07572, WO04024712, WO04024713, WO04087647, WO2005016916, WO2005097781, WO06010831, WO04035569, WO04089947, WO06021448, WO06032466, WO06038172, WO06046552, WO06099884, WO06105289, WO06126171, WO06125974, WO06134378, WO06137485, WO07016610, WO07081597, WO07071936, WO07115947, WO07118130, WO07122258, WO08006648, WO08003690 and WO08009700 disclose quinoline, naphthyridine, morpholine, cyclohexane, piperidine and piperazine derivatives having antibacterial activity.

This invention provides a compound of formula (I) or a pharmaceutically acceptable salt and/or N-oxide thereof:

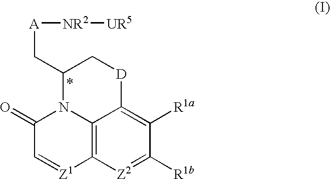

wherein:

### D is O, S or CH₂;

one of Z¹ and Z² is N or CR^{1c} and the other is independently CR^{1c};
R^{1a}, R^{1b} and R^{1c} are independently selected from hydrogen; halogen; cyano; (C₁₋₆)alkyl; (C₁₋₆)alkylthio; trifluoromethyl; trifluoromethoxy; carboxy; hydroxy optionally substituted with (C₁₋₆)alkyl or (C₁₋₆)alkoxy-substituted(C₁₋₆)alkyl; (C₁₋₆)alkoxy-substituted(C₁₋₆)alkyl; hydroxy (C₁₋₆)alkyl; an amino group optionally N-substituted by one or two (C₁₋₆)alkyl, formyl, (C₁₋₆)alkylcarbonyl or (C₁₋₆)alkylsulphonyl groups; and aminocarbonyl wherein the amino group is optionally substituted by (C₁₋₄)alkyl;
or when one of Z¹ and Z² is CR^{1c}, R^{1c} may instead be:
(C₃₋₆)cycloalkyl; (C₃₋₆)cycloalkoxy; (C₂₋₆)alkenyl optionally substituted by carboxy, (C₁₋₆)alkoxycarbonyl or aminocarbonyl wherein the amino group is optionally substituted by one or two (C₁₋₄)alkyl; (C₁₋₆)alkylcarbonyl; (C₁₋₆)alkylcarbonyl oxime; (C₁₋₄)alkyloxycarbonyl(C₁₋₆)alkyloxy; (C₁₋₄)alkylaminocarbonyl(C₁₋₆)alkyloxy; amino substituted by (C₁₋₄)alkylaminocarbonyl; aminocarbonyl wherein the amino group is substituted by (C₁₋₄)alkoxysulphonyl, hydroxy(C₁₋₄)alkyl, (C₁₋₄)alkoxy-substituted(C₁₋)alkyl, (C₃₋₆)cycloalkyl, phenyl, benzyl, monocyclic heteroaryl or monocyclic heteroaryl-methyl; benzyloxy; phenyl; benzyl; monocyclic heteroaryl; or monocyclic heteroaryl-methyl;
wherein heteroaryl is a 5 or 6 membered ring containing up to four hetero-atoms selected from oxygen, nitrogen and sulphur, and wherein a heteroaryl or phenyl ring in R^{1b} may be optionally C-substituted by up to three groups selected from (C₁₋₄)alkylthio; halo; carboxy(C₁₋₄)alkyl; halo(C₁₋₄)alkoxy; halo(C₁₋₄)alkyl; (C₁₋₄)alkyl; (C₂₋₄)alkenyl; (C₁₋₄)alkoxycarbonyl; formyl; (C₁₋₄)alkylcarbonyl; (C₂₋₄)alkenyloxycarbonyl; (C₂₋₄)alkenylcarbonyl; (C₁₋₄)alkylcarbonyloxy; (C₁₋₄)alkoxycarbonyl(C₁₋₄)alkyl; hydroxy; hydroxy(C₁₋₄)alkyl; mercapto(C₁₋₄)alkyl; (C₁₋₄)alkoxy; nitro; cyano; carboxy; amino or aminocarbonyl optionally substituted by one or two (C₁₋₄)alkyl; (C₁₋₄)alkylsulphonyl; (C₂₋₄)alkenylsulphonyl; or aminosulphonyl wherein the amino group is optionally substituted by (C₁₋₄)alkyl or (C₂₋₄)alkenyl;
R² is hydrogen, or (C₁₋₄)alkyl, or together with R⁶ forms Y as defined below;
A is a group (i):

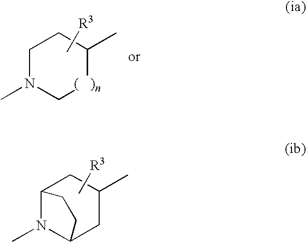

in which: R³ is as defined for R^{1a} or R^{1b}, provided that R³ in the 4-position is other than carboxy, or R³ is oxo and n is 1 or 2:
or A is a group (ii)

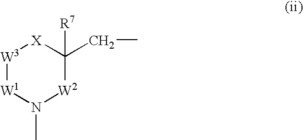

W¹, W² and W³ are CR⁴R⁸;

or W² and W³ are CR⁴R⁸ and W¹ represents a bond between W³ and N;

X is O, CR⁴R⁸, or NR⁶;

one R⁴ is as defined for R^{1a} and R^{1b} and the remainder and R⁸ are hydrogen or one R⁴ and R⁸ are together oxo and the remainder are hydrogen;

R⁶ is hydrogen or (C₁₋₆)alkyl; or together with R² forms Y;

R⁷ is hydrogen; halogen; hydroxy optionally substituted with (C₁₋₆)alkyl; or (C₁₋₆)alkyl;

Y is CR⁴R⁸CH₂; CH₂CR⁴R⁸; (C═O); CR⁴R⁸; CR⁴R⁸(C═O); or (C═O)CR⁴R⁸;

or when X is CR⁴R⁸, R⁸ and R⁷ together represent a bond;

U is selected from CO and CH₂ and
R⁵ is an optionally substituted bicyclic carbocyclic or heterocyclic ring system (B):

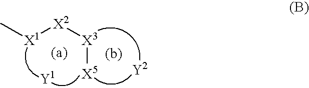

containing up to four heteroatoms in each ring in which

at least one of rings (a) and (b) is aromatic;

X¹ is C or N when part of an aromatic ring, or CR¹⁴ when part of a non-aromatic ring;

X² is N, NR¹³, O, S(O)_{X}, CO or CR¹⁴ when part of an aromatic or non-aromatic ring or may in addition be CR¹⁴R¹⁵ when part of a non aromatic ring;

X³ and X⁵ are independently N or C;

Y¹ is a 0 to 4 atom linker group each atom of which is independently selected from N, NR¹³, O, S(O)_{X}, CO and CR¹⁴ when part of an aromatic or non-aromatic ring or may additionally be CR¹⁴R¹⁵ when part of a non aromatic ring;

Y² is a 2 to 6 atom linker group, each atom of Y² being independently selected from N, NR¹³, O, S(O)_{X}, CO, CR¹⁴ when part of an aromatic or non-aromatic ring or may additionally be CR¹⁴R¹⁵ when part of a non aromatic ring;

each of R¹⁴ and R¹⁵ is independently selected from: H; (C₁₋₄)alkylthio; halo; carboxy(C₁₋₄)alkyl; (C₁₋₄)alkyl; (C₁₋₄)alkoxycarbonyl; (C₁₋₄)alkylcarbonyl; (C₁₋₄)alkoxy (C₁₋₄)alkyl; hydroxy; hydroxy(C₁₋₄)alkyl; (C₁₋₄)alkoxy; nitro; cyano; carboxy; amino or aminocarbonyl optionally mono- or di-substituted by (C₁₋₄)alkyl; or

R¹⁴ and R¹⁵ may together represent oxo;

each R¹³ is independently H; trifluoromethyl; (C₁₋₄)alkyl optionally substituted by hydroxy, (C₁₋₆)alkoxy, (C₁₋₆)alkylthio, halo or trifluoromethyl; (C₂₋₄)alkenyl; (C₁₋₄)alkoxycarbonyl; (C₁₋₄)alkylcarbonyl; (C₁₋₆)alkylsulphonyl; aminocarbonyl wherein the amino group is optionally mono or disubstituted by (C₁₋₄)alkyl; and

each x is independently 0, 1 or 2.

This invention also provides a method of treatment of bacterial infections including tuberculosis in mammals, particularly in man, which method comprises the administration to a mammal in need of such treatment an effective amount of a compound of formula (I), or a pharmaceutically acceptable salt and/or N-oxide thereof.

The invention also provides the use of a compound of formula (I), or a pharmaceutically acceptable salt and/or N-oxide thereof, in the manufacture of a medicament for use in the treatment of bacterial infections including tuberculosis in mammals.

The invention also provides a pharmaceutical composition comprising a compound of formula (I), or a pharmaceutically acceptable salt and/or N-oxide thereof, and a pharmaceutically acceptable carrier.

In a particular aspect when D is O or S, the stereochemistry at the carbon atom marked * is (R). In another particular aspect, when D is CH₂, the stereochemistry at the carbon atom marked * has the same absolute configuration as Example 16 3-({4-[(2,3-Dihydro[1,4]dioxino[2,3-c]pyridin-7-ylmethyl)amino]-1-piperidinyl}methyl)-10-fluoro-2,3-dihydro-5H-[1,4]oxazino[2,3,4-ij]quinolin-5-one Enantiomer 1.

In a particular aspect each R^{1a} and R^{1b} is independently hydrogen, (C₁₋₄)alkoxy, (C₁₋₄)alkylthio, (C₁₋₄)alkyl, cyano, carboxy, hydroxymethyl or halogen; more particularly hydrogen, methoxy, methyl, cyano, or halogen such as fluoro, chloro or bromo.

In certain embodiments only one group R^{1a}, R^{1b} and R^{1c} is other than hydrogen.

In particular embodiments Z¹ and Z² are both CH, R^{1a} is fluoro and R^{1b} is hydrogen.

In further embodiments Z¹ is CH and Z² is CR^{1c} where R^{1c} is other than hydrogen, in particular R^{1c} is cyano, —CH═CHCO₂H or —CH═CHCO₂C₂H₅, R^{1a} is fluoro and R^{1b} is hydrogen.

In other embodiments Z¹ is CH and Z² is N, R^{1a} is fluoro, bromo, cyano or methoxy and R^{1b} is hydrogen

In further embodiments Z¹ is N and Z² is CH and R^{1a} is chloro.

In a particular aspect R² is hydrogen.

Particular examples of R³ include hydrogen; optionally substituted hydroxy; optionally substituted amino; halogen; (C₁₋₄) alkyl; 1-hydroxy-(C₁₋₄) alkyl; optionally substituted aminocarbonyl. More particular R³ groups are hydrogen; CONH₂; 1-hydroxyalkyl e.g. CH₂OH; optionally substituted hydroxy e.g. methoxy; optionally substituted amino; and halogen, in particular fluoro. Most particularly R³ is hydrogen, hydroxy or fluoro.

In a particular aspect, when A is (ia), n is 1. In a further aspect R³ is in the 3- or 4-position. In a more particular aspect, A is (ia), n is 1 and R³ is in the 3-position, and more particularly is cis to the NR² group.

In particular embodiments, A is a group (ia) in which n is 1 and R³ is hydrogen or hydroxy. More particularly, where A is 3-hydroxy-piperidin-4-yl the configuration is (3R,4S) or (3S,4R).

Alternatively and more particularly where A is 3-hydroxypiperidin-4-yl the configuration is (3R,4S).

In a particular aspect, when A is (ii), X is CR⁴R⁸, R⁸ is H and R⁴ is H or OH and more particularly OH is trans to R⁷. In a further aspect W¹ is a bond. In another aspect R⁷ is H. In an additional aspect W¹ is a bond, W² and W³ are both CH₂ and R⁷ is H. Where A is 4-hydroxypyrrolidin-3-ylmethyl, in a particular aspect the configuration is (3S,4S).

In a particular aspect, when A is (ii), X is O, R⁷ is H and W¹, W² and W³ are each CH₂.

In certain embodiments U is CH₂.

In certain embodiments R⁵ is an aromatic heterocyclic ring (B) having 8-11 ring atoms including 2-4 heteroatoms of which at least one is N or NR¹³ in which, in particular embodiments, Y² contains 2-3 heteroatoms, one of which is S and 1-2 are N, with one N bonded to X³.

In alternative embodiments the heterocyclic ring (B) has ring (a) aromatic selected from optionally substituted benzo, pyrido, pyridazino and pyrimidino and ring (b) non aromatic and Y² has 3-5 atoms, more particularly 4 atoms, including at least one heteroatom, with O, S, CH₂ or NR¹³ bonded to X⁵ where R¹³ is other than hydrogen, and either NHCO bonded via N to X³, or O, S, CH₂ or NH bonded to X³. In a particular aspect the ring (a) contains aromatic nitrogen, and more particularly ring (a) is pyridine or pyrazine. Examples of rings (B) include optionally substituted:

### (a) and (b) Aromatic

1H-pyrrolo[2,3-b]-pyridin-2-yl, 1H-pyrrolo[3,2-b]-pyridin-2-yl, 3H-imidazo[4,5-b]-pyrid-2-yl, 3H-quinazolin-4-one-2-yl, benzimidazol-2-yl, benzo[1,2,3]-thiadiazol-5-yl, benzo[1,2,5]-oxadiazol-5-yl, benzofur-2-yl, benzothiazol-2-yl, benzo[b]thiophen-2-yl, benzoxazol-2-yl, chromen-4-one-3-yl, imidazo[1,2-a]pyridin-2-yl, imidazo-[1,2-a]-pyrimidin-2-yl, indol-2-yl, indol-6-yl, isoquinolin-3-yl, [1,8]-naphthyridine-3-yl, oxazolo[4,5-b]-pyridin-2-yl, quinolin-2-yl, quinolin-3-yl, quinoxalin-2-yl, naphthalen-2-yl, 1,3-dioxo-isoindol-2-yl, 1H-benzotriazol-5-yl, 1H-indol-5-yl, 3H-benzooxazol-2-one-6-yl, 3H-benzooxazol-2-thione-6-yl, 3H-benzothiazol-2-one-5-yl, 3H-quinazolin-4-one-6-yl, pyrido[1,2-a]pyrimidin-4-one-3-yl (4-oxo-4H-pyrido[1,2-a]pyrimidin-3-yl), benzo[1,2,3]thiadiazol-6-yl, benzo[1,2,5]thiadiazol-5-yl, benzo[1,4]oxazin-2-one-3-yl, benzothiazol-5-yl, benzothiazol-6-yl, cinnolin-3-yl, imidazo[1,2-b]pyridazin-2-yl, pyrazolo[1,5-a]pyrazin-2-yl, pyrazolo[1,5-a]pyridin-2-yl, pyrazolo[1,5-a]pyrimidin-6-yl, pyrazolo[5,1-c][1,2,4]triazin-3-yl, pyrido[1,2-a]pyrimdin-4-one-2-yl (4-oxo-4H-pyrido[1,2-a]pyrimidin-2-yl), quinazolin-2-yl, quinoxalin-6-yl, thiazolo[3,2-a]pyrimidin-5-one-7-yl, thiazolo[5,4-b]pyridin-2-yl, thieno[3,2-b]pyridin-6-yl, thiazolo[5,4-b]pyridin-6-yl, thiazolo[4,5-b]pyridin-5-yl, [1,2,3]thiadiazolo[5,4-b]pyridin-6-yl, 2H-isoquinolin-1-one-3-yl (1-oxo-1,2-dihydro-isoquinolin-3-yl), [1,2,4]triazolo[1,5-a]pyridin-6-yl

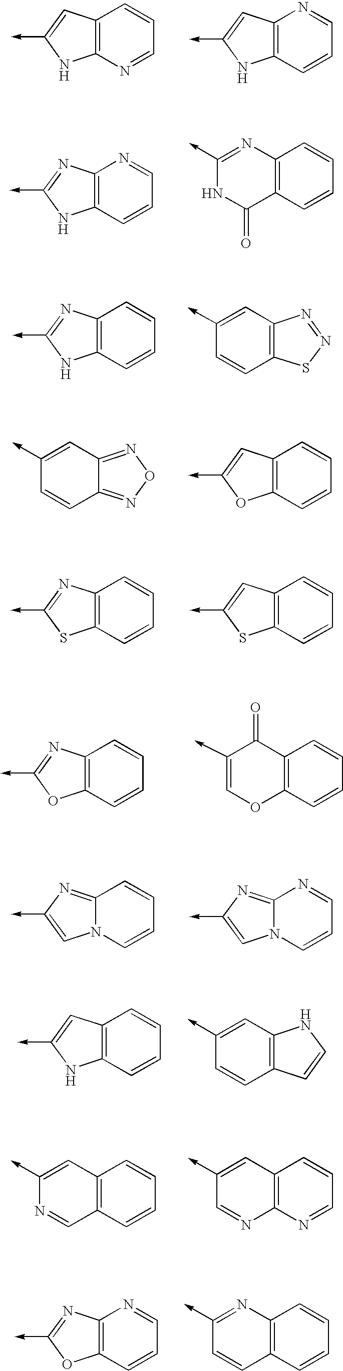

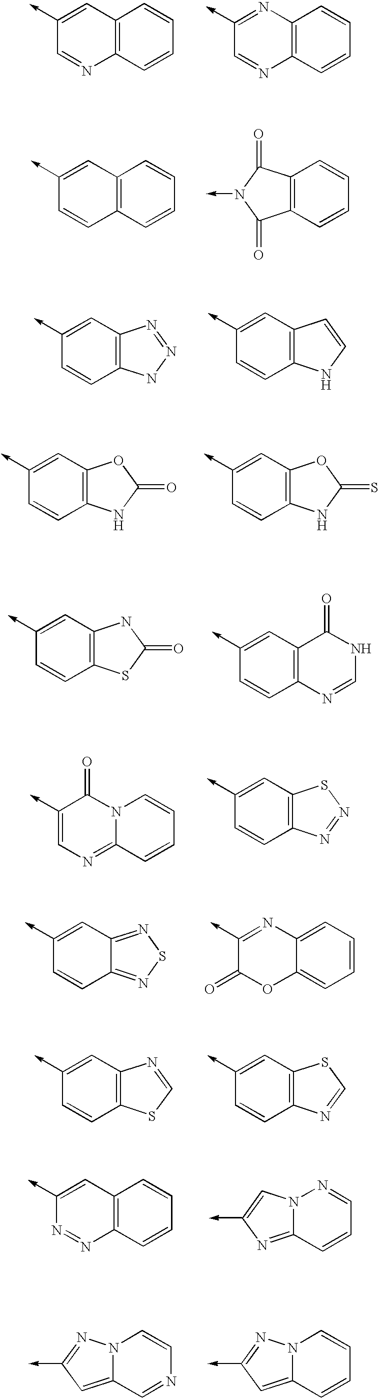

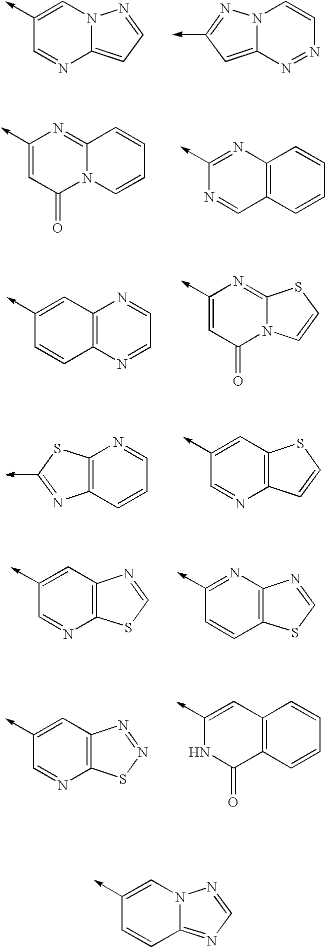

→is the point of attachment

### (a) is Non Aromatic

(2S)-2,3-dihydro-1H-indol-2-yl, (2S)-2,3-dihydro-benzo[1,4]dioxine-2-yl, 3-(R,S)-3,4-dihydro-2H-benzo[1,4]thiazin-3-yl, 3-(R)-2,3-dihydro-[1,4]dioxino[2,3-b]pyridin-3-yl, 3-(S)-2,3-dihydro-[1,4]dioxino[2,3-b]pyridin-3-yl, 2,3-dihydro-benzo[1,4]dioxan-2-yl, 3-substituted-3H-quinazolin-4-one-2-yl,

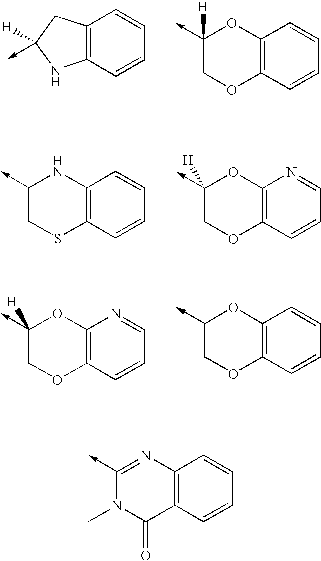

→is the point of attachment

### (b) is Non Aromatic

1,1,3-trioxo-1,2,3,4-tetrahydro 1 l⁶-benzo[1,4]thiazin-6-yl, benzo[1,3]dioxol-5-yl, 2,3-dihydro-benzo[1,4]dioxin-6-yl, 3-substituted-3H-benzooxazol-2-one-6-yl, 3-substituted-3H-benzooxazole-2-thione-6-yl, 3-substituted-3H-benzothiazol-2-one-6-yl, 4H-benzo[1,4]oxazin-3-one-6-yl (3-oxo-3,4-dihydro-2H-benzo[1,4]oxazin-6-yl), 4H-benzo[1,4]thiazin-3-one-6-yl (3-oxo-3,4-dihydro-2H-benzo[1,4]thiazin-6-yl), 4H-benzo[1,4]oxazin-3-one-7-yl, 4-oxo-2,3,4,5-tetrahydro-benzo[b][1,4]thiazepine-7-yl, 5-oxo-2,3-dihydro-5H-thiazolo[3,2-a]pyrimidin-6-yl, 1H-pyrido[2,3-b][1,4]thiazin-2-one-7-yl (2-oxo-2,3-dihydro-1H-pyrido[2,3-b]thiazin-7-yl), 2,3-dihydro-1H-pyrido[2,3-b][1,4]thiazin-7-yl, 2-oxo-2,3-dihydro-1H-pyrido[3,4-b]thiazin-7-yl, 2,3-dihydro-[1,4]dioxino[2,3-b]pyridin-6-yl, 2,3-dihydro-[1,4]dioxino[2,3-c]pyridin-7-yl, 2,3-dihydro-[1,4]dioxino[2,3-b]pyridin-7-yl, 3,4-dihydro-2H-benzo[1,4]oxazin-6-yl, 3,4-dihydro-2H-benzo[1,4]thiazin-6-yl, 3-oxo-3,4-dihydro-2H-pyrido[3,2-b][1,4]oxazin-6-yl, 3,4-dihydro-2H-pyrido[3,2-b][1,4]thiazin-6-yl, 3-oxo-3,4-dihydro-2H-pyrido[3,2-b][1,4]thiazin-6-yl, 3,4-dihydro-1H-quinolin-2-one-7-yl, 3,4-dihydro-1H-quinoxalin-2-one-7-yl, 6,7-dihydro-4H-pyrazolo[1,5-a]pyrimidin-5-one-2-yl, 5,6,7,8-tetrahydro-[1,8]naphthyridin-2-yl (1,2,3,4-tetrahydro-[1,8]naphthyridin-7-yl), 2-oxo-3,4-dihydro-1H-[1,8]naphthyridin-6-yl, 6-oxo-6,7-dihydro-5H-pyridazino[3,4-b][1,4]thiazin-3-yl (6-oxo-6,7-dihydro-5H-8-thia-1,2,5-triaza-naphthalen-3-yl), 2-oxo-2,3-dihydro-1H-pyrido[3,4-b][1,4]oxazin-7-yl, 2-oxo-2,3-dihydro-1H-pyrido[2,3-b][1,4]oxazin-7-yl, 6,7-dihydro-[1,4]dioxino[2,3-d]pyrimidin-2-yl, [1,3]oxathiolo[5,4-c]pyridin-6-yl, 3,4-dihydro-2H-pyrano[2,3-c]pyridine-6-yl, 2,3-dihydro[1,4]oxathiino[2,3-c]pyridine-7-yl, 6,7-dihydro[1,4]oxathiino[2,3-c]pyridazin-3-yl, 6,7-dihydro[1,4]dioxino[2,3-c]pyridazin-3-yl, 2,3-dihydrofuro[2,3-c]pyridine-5-yl, 5,6,7,8-tetrahydro-3-cinnolinyl, 6,7-dihydro-5H-cyclopenta[c]pyridazine-3-yl, 7,8-dihydro-5H-pyrano[4,3-c]pyridazine-3-yl, 6,7-dihydro-5H-pyrano[2,3-c]pyridazin-3-yl, 5,6-dihydrofuro[2,3-c]pyridazin-3-yl, 2,3-dihydrofuro[2,3-c]pyridin-5-yl
2-substituted 1H-pyrimido[5,4-b][1,4]oxazin-7(6H)-one, 2-substituted 5,6-dihydropyrido[2,3-d]pyrimidin-7(1H)-one, 7-substituted 2H-chromen-2-one, 7-substituted 2H-pyrano[2,3-b]pyridin-2-one, 2-substituted 6,7-dihydro-5H-pyrano[2,3-d]pyrimidine, 8-substituted 2H-pyrido[1,2-a]pyrimidin-2-one, 2,3-dihydro-1-benzofuran-5-yl, 2,3-dihydro-1H-pyrido[3,4-b][1,4]oxazin-7-yl

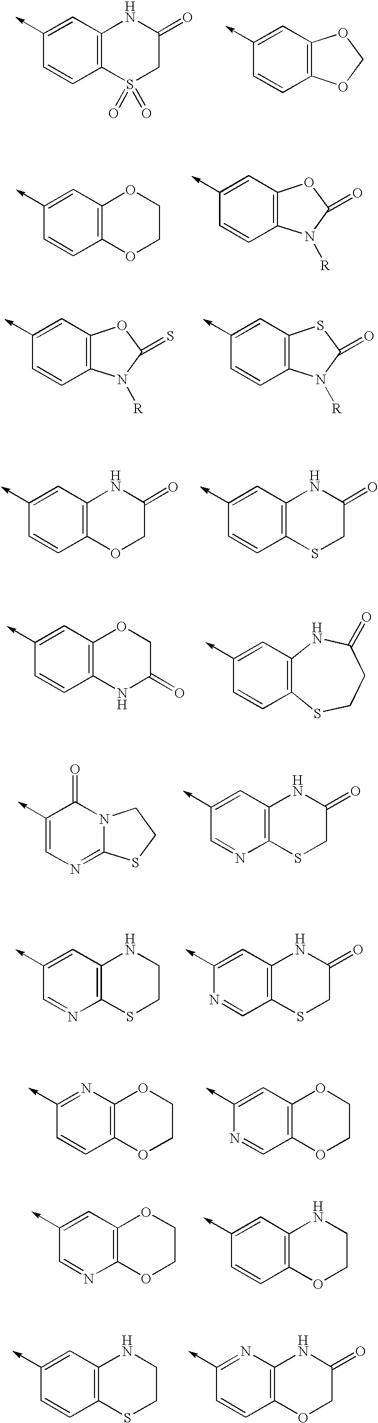

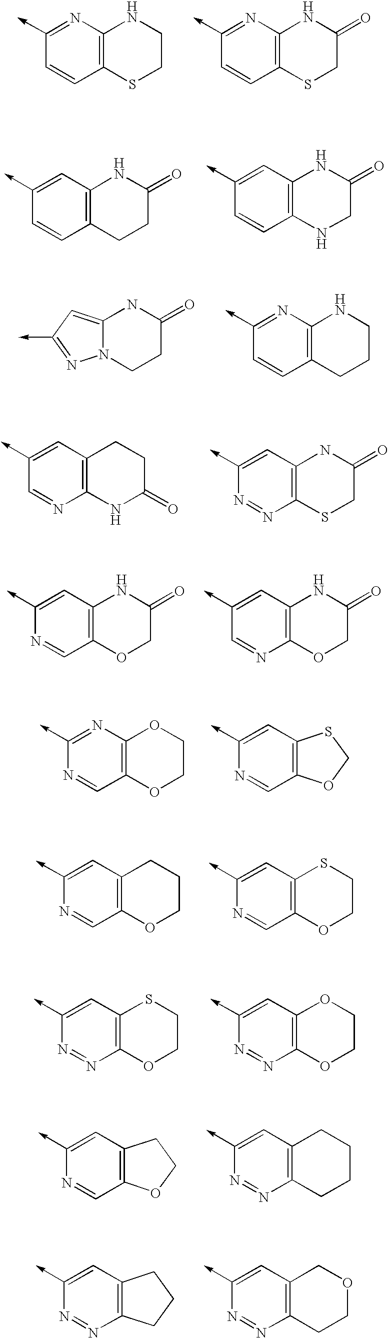

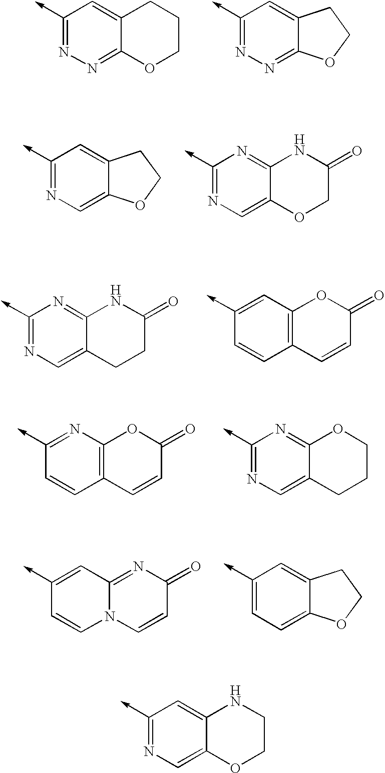

where R is an optional substituent
→is the point of attachment

In some embodiments R¹³ is H if in ring (a) or in addition (C₁₋₄)alkyl such as methyl or isopropyl when in ring (b). More particularly, in ring (b) R¹³ is H when NR¹³ is bonded to X³ and (C₁₋₄)alkyl when NR¹³ is bonded to X⁵.

In further embodiments R¹⁴ and R¹⁵ are independently selected from hydrogen, halo, hydroxy, (C₁₋₄) alkyl, (C₁₋₄)alkoxy, nitro and cyano. More particularly R¹⁵ is hydrogen.

More particularly each R¹⁴ is selected from hydrogen, chloro, fluoro, hydroxy, methyl, methoxy, nitro and cyano. Still more particularly R¹⁴ is selected from hydrogen, fluorine or nitro.

Most particularly R¹⁴ and R¹⁵ are each H.

Particular groups R⁵ include:
[1,2,3]thiadiazolo[5,4-b]pyridin-6-yl
1H-pyrrolo[2,3-b]pyridin-2-yl
2,3-dihydro-[1,4]dioxino[2,3-b]pyridin-6-yl
2,3-dihydro-[1,4]dioxino[2,3-b]pyridin-7-yl
2,3-dihydro-[1,4]dioxino[2,3-c]pyridin-7-yl
2,3-dihydro-benzo[1,4]dioxin-6-yl
2-oxo-2,3-dihydro-1H-pyrido[2,3-b][1,4]oxazin-7-yl
2-oxo-2,3-dihydro-1H-pyrido[2,3-b][1,4]thiazin-7-yl
3,4-dihydro-2H-benzo[1,4]oxazin-6-yl
3-methyl-2-oxo-2,3-dihydro-benzooxazol-6-yl
3-oxo-3,4-dihydro-2H-benzo[1,4]oxazin-6-yl
3-oxo-3,4-dihydro-2H-pyrido[3,2-b][1,4]oxazin-6-yl
3-oxo-3,4-dihydro-2H-benzo[1,4]thiazin-6-yl (4H-benzo[1,4]thiazin-3-one-6-yl)
4-oxo-4H-pyrido[1,2-a]pyrimidin-2-yl
6-nitro-benzo[1,3]dioxol-5-yl
7-fluoro-3-oxo-3,4-dihydro-2H-benzo[1,4]oxazin-6-yl
8-hydroxy-1-oxo-1,2-dihydro-isoquinolin-3-yl
8-hydroxyquinolin-2-yl
benzo[1,2,3]thiadiazol-5-yl
benzo[1,2,5]thiadiazol-5-yl
benzothiazol-5-yl
thiazolo-[5,4-b]pyridin-6-yl
3-oxo-3,4-dihydro-2H-pyrido[3,2-b][1,4]thiazin-6-yl
7-chloro-3-oxo-3,4-dihydro-2H-pyrido[3,2-b][1,4]thiazin-6-yl
7-chloro-3-oxo-3,4-dihydro-2H-pyrido[3,2-b][1,4]oxazin-6-yl
7-fluoro-3-oxo-3,4-dihydro-2H-pyrido[3,2-b][1,4]thiazin-6-yl
2-oxo-2,3-dihydro-1H-pyrido[3,4-b][1,4]thiazin-7-yl
[1,3]oxathiolo[5,4-c]pyridin-6-yl
3,4-dihydro-2H-pyrano[2,3-c]pyridin-6-yl
5-carbonitro-2,3-dihydro-1,4-benzodioxin-7-yl
2,3-dihydro[1,4]oxathiino[2,3-c]pyridin-7-yl
6,7-dihydro[1,4]oxathiino[2,3-c]pyridazin-3-yl
6,7-dihydro[1,4]dioxino[2,3-c]pyridazin-3-yl
2,3-Dihydrofuro[2,3-c]pyridine-5-yl
5,6,7,8-tetrahydro-3-cinnolinyl
6,7-dihydro-5H-cyclopenta[c]pyridazine-3-yl
7,8-dihydro-5H-pyrano[4,3-c]pyridazine-3-yl
[1,2,4]triazolo[1,5-a]pyridin-6-yl
6-fluoro-2,3-dihydro-1,4-benzodioxin-7-yl
2,3-dihydro-1-benzofuran-5-yl
6,7-dihydro-5H-pyrano[2,3-c]pyridazin-3-yl
2-substituted 1H-pyrimido[5,4-b][1,4]oxazin-7(6H)-one
2-substituted 4-chloro-1H-pyrimido[5,4-b][1,4]oxazin-7(6H)-one
2-substituted 5,6-dihydropyrido[2,3-d]pyrimidin-7(1H)-one
2-substituted 4-chloro-5,6-dihydropyrido[2,3-d]pyrimidin-7(1H)-one
2-substituted 4-methyl-5,6-dihydropyrido[2,3-d]pyrimidin-7(1H)-one
2-substituted 4-methyloxy-5,6-dihydropyrido[2,3-d]pyrimidin-7(1H)-one
7-substituted 2H-chromen-2-one
7-substituted 2H-pyrano[2,3-b]pyridin-2-one
4-chloro-6,7-dihydro-5H-pyrano[2,3-d]pyrimidin-2-yl
8-substituted 2H-pyrido[1,2-a]pyrimidin-2-one
6,7-dihydro-5H-pyrano[2,3-d]pyrimidin-2-yl)
5-chloro-1-benzothiophen-2-yl
6-chloro-1-benzothiophen-2-yl
1-benzothiophen-5-yl
1-methyl-1H-1,2,3-benzotriazol-6-yl
imidazo[2,1-b][1,3]thiazol-6-yl
4-methyl-3,4-dihydro-2H-1,4-benzoxazin-7-yl
1-methyl-1H-indol-2-yl
2,3-dihydro-1H-pyrido[3,4-b][1,4]oxazin-7-yl

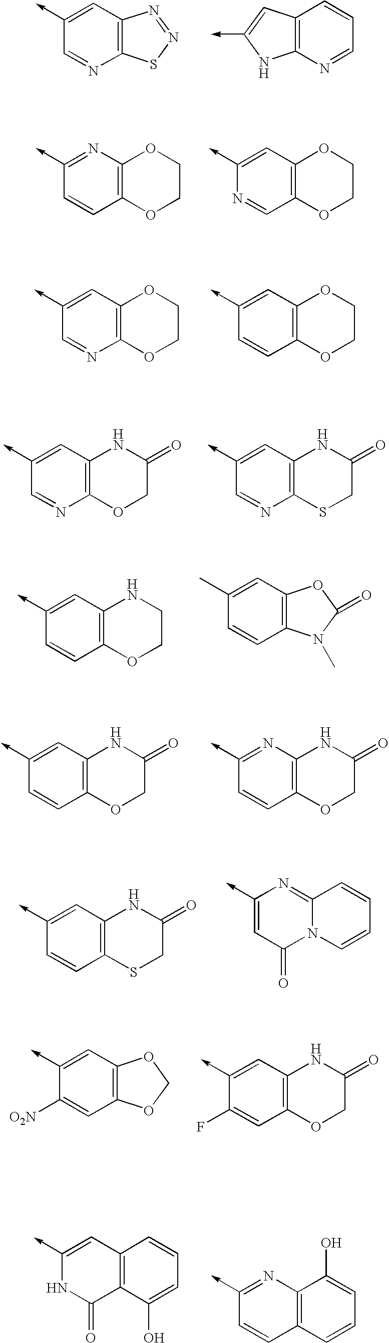

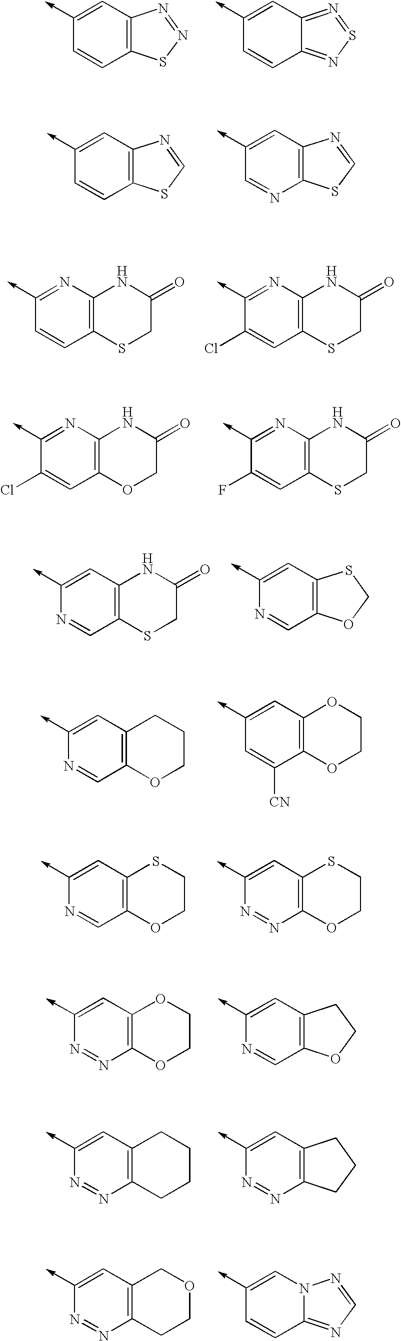

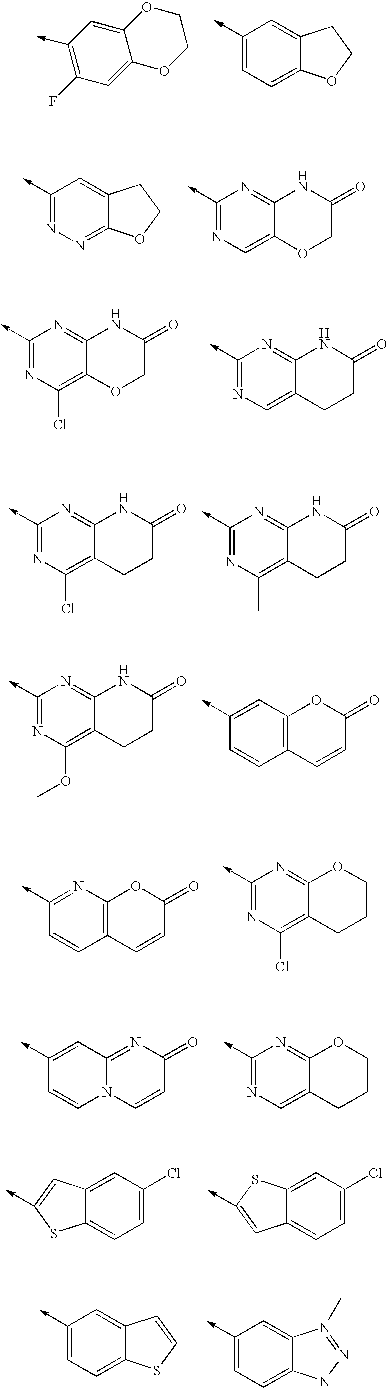

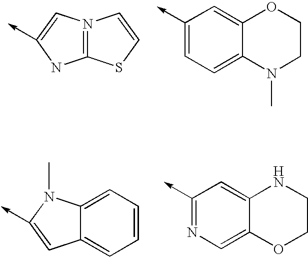

→is the point of attachment
especially
3-oxo-3,4-dihydro-2H-pyrido[3,2-b][1,4]oxazin-6-yl
3-oxo-3,4-dihydro-2H-pyrido[3,2-b][1,4]thiazin-6-yl
6,7-dihydro[1,4]dioxino[2,3-c]pyridazin-3-yl
6,7-dihydro[1,4]oxathiino[2,3-c]pyridazin-3-yl
2,3-dihydro-[1,4]dioxino[2,3-c]pyridin-7-yl
[1,3]oxathiolo[5,4-c]pyridin-6-yl
3,4-dihydro-2H-pyrano[2,3-c]pyridin-6-yl
2,3-dihydro[1,4]oxathiino[2,3-c]pyridin-7-yl

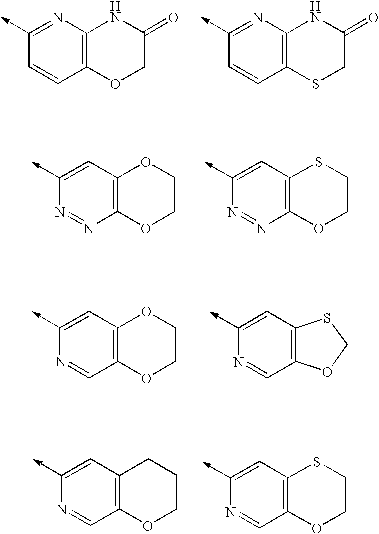

→is the point of attachment

When used herein, the term “alkyl” includes groups having straight and branched chains, for instance, and as appropriate, methyl, ethyl, n-propyl, iso-propyl, n-butyl, iso-butyl, sec-butyl, t-butyl, pentyl and hexyl. The term ‘alkenyl’ should be interpreted accordingly.

Halo or halogen includes fluoro, chloro, bromo and iodo.

Haloalkyl moieties include 1-3 halogen atoms.

Compounds within the invention contain a heterocyclyl group and may occur in two or more tautomeric forms depending on the nature of the heterocyclyl group; all such tautomeric forms are included within the scope of the invention.

Some of the compounds of this invention may be crystallised or recrystallised from solvents such as aqueous and organic solvents. In such cases solvates may be formed. This invention includes within its scope stoichiometric solvates including hydrates as well as compounds containing variable amounts of water that may be produced by processes such as lyophilisation.

Furthermore, it will be understood that phrases such as “a compound of formula (I) or a pharmaceutically acceptable salt or N-oxide thereof” are intended to encompass the compound of formula (I), an N-oxide of formula (I), a pharmaceutically acceptable salt of the compound of formula (I) or any pharmaceutically acceptable combination of these. By way of non-limiting example used here for illustrative purpose, “a compound of formula (I) or a pharmaceutically acceptable salt thereof” may include a pharmaceutically acceptable salt of a compound of formula (I) that is further present as a solvate.

Since the compounds of formula (I) are intended for use in pharmaceutical compositions it will readily be understood that in particular embodiments they are provided in substantially pure form, for example at least 60% pure, more suitably at least 75% pure and particularly at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and more particularly from 10% of a compound of the formula (I) or pharmaceutically acceptable salt and/or N-oxide thereof.

Particular compounds according to the invention include those mentioned in the examples and their pharmaceutically acceptable salts or N-oxides.

Pharmaceutically acceptable salts of the above-mentioned compounds of formula (I) include the acid addition or quaternary ammonium salts, for example their salts with mineral acids e.g. hydrochloric, hydrobromic, sulphuric nitric or phosphoric acids, or organic acids, e.g. acetic, fumaric, succinic, maleic, citric, benzoic, p-toluenesulphonic, methanesulphonic, naphthalenesulphonic acid or tartaric acids. Compounds of formula (I) may also be prepared as the N-oxide. The invention extends to all such derivatives.

Certain of the compounds of formula (I) may exist in the form of optical isomers, e.g. diastereoisomers and mixtures of isomers in all ratios, e.g. racemic mixtures. The invention includes all such forms, in particular the pure isomeric forms. For example the invention includes enantiomers and diastereoisomers at the attachment point of NR² and R³. The different isomeric forms may be separated or resolved one from the other by conventional methods, or any given isomer may be obtained by conventional synthetic methods or by stereospecific or asymmetric syntheses. Certain compounds of formula (I) may also exist in polymorphic forms and the invention includes such polymorphic forms.

In a further aspect of the invention there is provided a process for preparing compounds of formula (I), and pharmaceutically acceptable salt and/or N-oxide thereof, which process comprises reacting a compound of formula (IIA) with a compound of formula (III):

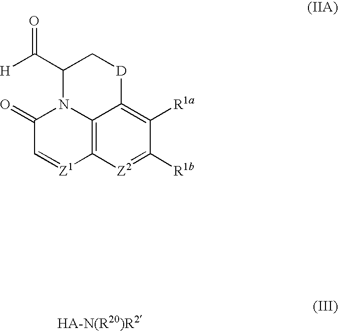

in which:
R²⁰ is UR⁵ or a group convertible thereto and R^{2′} is R² or a group convertible thereto, wherein Z¹ and Z², A, R^{1a}, R^{1b}, R², U and R⁵ are as defined in formula (I), and thereafter optionally or as necessary converting R²⁰ and R^{2′} to UR⁵ and R², interconverting any variable groups, and/or forming a pharmaceutically acceptable salt or N-oxide thereof.

The reaction is a reductive alkylation (see for examples Smith, M. B.; March, J. M. Advanced Organic Chemistry, Wiley-Interscience 2001) with a suitable reducing agent such as sodium cyanoborohydride (in methanol/chloroform/acetic acid), triacetoxyborohydride or (polystyrylmethyl)trimethylammonium cyanoborohydride. If the amine is present as a hydrochloride salt it is preferable to have an excess of sodium acetate present to buffer the reaction. 3 A Molecular sieves may also be used to help formation of the initial imine intermediate. The compound of formula (IIA) may be presented as a hemiacetal.

In a further aspect of the invention there is provided a process for preparing compounds of formula (I), and pharmaceutically acceptable salt and/or N-oxide thereof, which process comprises reacting a compound of formula (IIB) with a compound of formula (III):

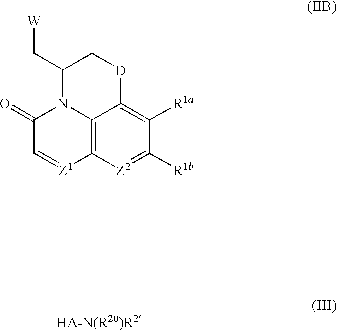

W is a leaving group, R²⁰ is UR⁵ or a group convertible thereto and R^{2′} is R² or a group convertible thereto, wherein Z¹ and Z², A, R^{1a}, R^{1b}, R², U and R⁵ are as defined in formula (I), and thereafter optionally or as necessary converting R²⁰ and R^{2′} to UR⁵ and R², interconverting any variable groups, and/or forming a pharmaceutically acceptable salt or N-oxide thereof.

The leaving group W may be any conventional group such as methanesulfonyl or methylbenzenesulfonyl. The reaction is carried out under conventional conditions for amine coupling such as reacting together in the presence of a suitable base, such as pyridine or triethylamine, in a suitable solvent such as acetonitrile, ethanol or N,N-dimethylformamide at temperatures between ambient and 80° C.

The reaction of (IIA) or (IIB) and (III) gives a compound of formula (IIC):

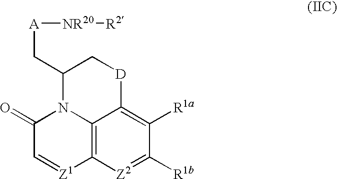

The invention further provides compounds of formula (IIC) in which R²⁰ is hydrogen.

In the reactions above, conveniently one of R²⁰ and R^{2′} is an N-protecting group, such as such as t-butoxycarbonyl, benzyloxycarbonyl, 9-fluorenylmethyloxycarbonyl or trifluoroacetyl. This may be removed by several methods well known to those skilled in the art (for examples see “Protective Groups in Organic Synthesis, T. W. Greene and P. G. M. Wuts, Wiley-Interscience, 1999), for example conventional acid hydrolysis (e.g. trifluoroacetic acid/dichloromethane, hydrochloric acid/dichloromethane/methanol), or potassium carbonate/methanol.

The free amine of formula (IIC) in which R²⁰ is hydrogen may be converted to NR²UR⁵ by conventional means such as amide formation with an acyl derivative R⁵COW, for compounds where U is CO or, where U is CH₂, by alkylation with an alkyl halide R⁵CH₂-halide in the presence of base, acylation/reduction with an acyl derivative R⁵COW or reductive alkylation with an aldehyde R⁵CHO under conventional conditions (see for examples Smith, M. B.; March, J. M. Advanced Organic Chemistry, Wiley-Interscience 2001). Suitable conditions include sodium cyanoborohydride (in methanol/chloroform/acetic acid) or (polystyrylmethyl)trimethylammonium cyanoborohydride. If the amine (III) is a hydrochloride salt then sodium acetate may be added to buffer the reaction. Sodium triacetoxyborohydride is an alternative reducing agent.

The appropriate reagents containing the required R⁵ group are known compounds or may be prepared analogously to known compounds, see for example WO02/08224, WO02/50061, WO02/56882, WO02/96907, WO2003087098, WO2003010138, WO2003064421, WO2003064431, WO2004002992, WO2004002490, WO2004014361, WO2004041210, WO2004096982, WO2002050036, WO2004058144, WO2004087145, WO2006014580, WO2004/035569, WO2004/089947, WO2003082835, WO06002047, WO06014580, WO06010040, WO06017326, WO06012396, WO06017468, WO06020561, WO2002026723, WO06132739, WO06134378, WO06137485, WO06081179, WO06081264, WO06081289, WO06081178, WO06081182, WO07016610, WO07081597, WO07071936, WO07115947, WO07118130, WO07122258, WO08006648, WO08003690, WO08009700, WO2007067511 and EP0559285.

Where R⁵ contains an NH group, this may be protected with a suitable N-protecting group such as t-butoxycarbonyl, benzyloxycarbonyl or 9-fluorenylmethyloxycarbonyl during the coupling of the R⁵ derivative with the free amine of formula (IIB). The protecting group may be removed by conventional methods, such as by treatment with trifluoroacetic acid.

Compounds of formula (IIA) may be prepared from compounds of formula (IIB) where W is OH by oxidation under conventional conditions, (see for examples Smith, M. B.; March, J. M. Advanced Organic Chemistry, Wiley-Interscience 2001). Suitable conditions include oxidants based on DMSO and oxalyl chloride, or hypervalent iodine reagents, such as Dess-Martin periodinane (1,1,1-tris(acetyloxy)-1,1-dihydro-1,2-benziodoxol-3-(1H)-one.

Compounds of formula (IIB) where W is OH may be prepared by treatment of compounds of formula (IV):

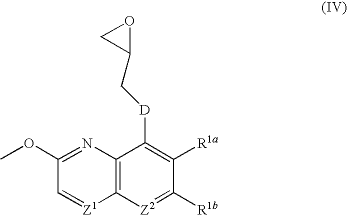

with Lewis acid such as aluminium trichloride, or ytterbium triflate in a suitable solvent such as dichloromethane, THF, or mixtures thereof. The resulting alcohol may be activated by treatment with methansulfonic acid or other activating agent. Compounds of formula (IV) where D is O or S can be prepared as follows:

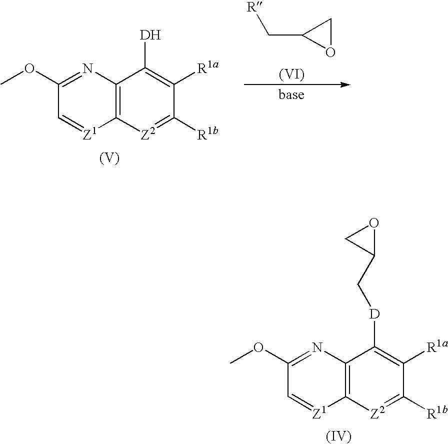

by reacting alcohol or thiol (V) with epoxide (VI) where R″ is a leaving group e.g. halo, 4-methylbenzenesulfonate or 3-nitrobenzene sulfonate under conventional conditions (see for examples Smith, M. B.; March, J. M. Advanced Organic Chemistry, Wiley-Interscience 2001). Suitable conditions include the presence of a base, for example sodium hydride, or potassium carbonate, in a solvent such as DMF. In some instances when both Z¹ and Z² ares CH, alkylation of (V) with (VI) proceeds through (IV) without isolation of (IV) directly to (IIB). The epoxide (VI) can be racemic or optically active. When (VI) is optically active this gives optically active (IV) and hence (IIB).

Compounds of formula (IV) where D is CH₂ can be prepared as follows:

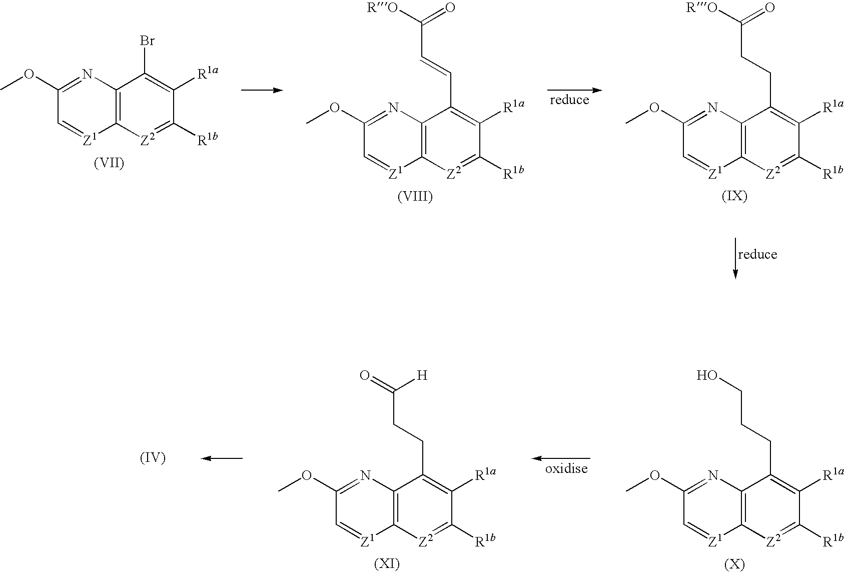

Compounds of formula (VII) can be converted to compounds of formula (VIII) where R′″ is an alkyl group under conventional palladium-catalysed methods of arylation of alkenes (see for examples Smith, M. B.; March, J. M. Advanced Organic Chemistry, Wiley-Interscience 2001). Suitable reagents for the reaction of (VII) with an R′″ ester of acrylic acid include the palladium catalysts tris(dibenzylideneacetone)dipalladium(0) and bis(triphenylphosphine)palladium(0) in a suitable solvent such as 1,4-dioxane in the presence of base such as dicyclohexylmethylamine at elevated temperature, suitably 70° C. Compounds of formula (VIII) can be converted to compounds of formula (IX) by reduction under conventional conditions (see for examples Smith, M. B.; March, J. M. Advanced Organic Chemistry, Wiley-Interscience 2001). Suitable conditions include hydrogenation at atmospheric pressure over a catalyst such as 5% Pd/C in a suitable solvent such as ethanol, methanol or 1,4-dioxane. Compounds of formula (IX) can be converted to compounds of formula (X) by reduction under conventional conditions (see for examples Smith, M. B.; March, J. M. Advanced Organic Chemistry, Wiley-Interscience 2001). Suitable conditions include hydride reducing agents such as lithium aluminium hydride in a suitable solvent such as diethyl ether or THF. Compounds of formula (X) can be oxidised to compounds of formula (XI) by oxidation under conventional conditions (see for examples Smith, M. B.; March, J. M. Advanced Organic Chemistry, Wiley-Interscience 2001). Suitable conditions include oxidants based on DMSO and oxalyl chloride, or hypervalent iodine reagents such as Dess-Martin periodinane (1,1,1-tris(acetyloxy)-1,1-dihydro-1,2-benziodoxol-3-(1H)-one), or the oxidising agent reagent known as IBX (cyclic tautomer of 2-iodoxy benzoic acid) (D. B. Dess and J. C. Martin, J. Org. Chem. 1983, 48, 4155-4156). Compounds of formula (XI) may be converted into compounds of formula (IV) (where D=CH₂) on treatment with dimethylsulfoxonium methylide according to standard methods (for example see A. W, Beck et al, J. Chem Soc Perkin 1, 1990, 689).

In an alternative synthesis of compounds of formula (IIC), compounds of formula (IV) may be converted to compounds of formula (XII) by heating with an amine of formula (III) in a solvent such as acetonitrile, or DMF at temperatures from 50-130° C. Compounds of formula (XII) may be converted to compounds of formula (IIC) by treatment with methane sulfonic anhydride in the presence of base such as di-isopropyl ethylamine in a suitable solvent such as dichloromethane, chloroform, or 1,2-dichloroethane at temperatures between 40-100° C.

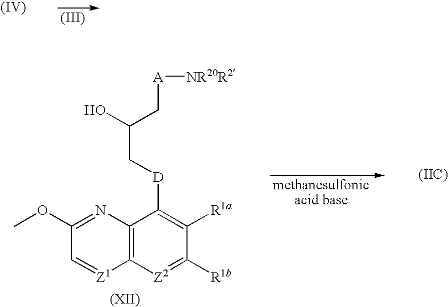

Interconversions of Z¹, Z², R^{1a}, R^{1b}, R^{1c}, R², A and R⁵ are conventional. In compounds which contain an optionally protected hydroxy group, suitable conventional hydroxy protecting groups which may be removed without disrupting the remainder of the molecule include acyl and alkylsilyl groups. N-protecting groups are removed by conventional methods.

Interconversion of R^{1a}, R^{1b} and R^{1c} groups may be carried out conventionally, on compounds of formula (I), (IIA), (IIB) or (IIC). For example R^{1a} or R^{1b} methoxy is convertible to R^{1a} or R^{1b} hydroxy by treatment with lithium and diphenylphosphine (general method described in Ireland et al, J. Amer. Chem. Soc., 1973, 7829) or HBr. Alkylation of the hydroxy group with a suitable alkyl derivative bearing a leaving group such as halide, yields R^{1a} or R^{1b} substituted alkoxy. R^{1a} halogen is convertible to other R^{1a} by conventional means, for example to hydroxy, alkylthiol (via thiol) and amino using metal catalysed coupling reactions, for example using copper as reviewed in Synlett (2003), 15, 2428-2439 and Angewandte Chemie, International Edition, 2003, 42(44), 5400-5449. R^{1a} fluoro may be converted to methoxy by treatment with sodium methoxide in a suitable solvent such as methanol and optionally dichloromethane. R^{1a} or R^{1b} halo such as bromo may be converted to cyano by treatment with copper (I) cyanide in N,N-dimethylformamide. R^{1a} or R^{1b} carboxy may be obtained by conventional hydrolysis of R^{1a} or R^{1b} cyano, and the carboxy converted to hydroxymethyl by conventional reduction.

Compounds of formulae (V) and (VII) are known compounds or can be prepared analogously, see for example WO2004/035569, WO2004/089947, WO02/08224, WO02040474, WO02072572, WO02/50061, WO02/56882, WO02/96907, WO2003087098, WO2003010138, WO2003064421, WO2003064431, WO2004002992, WO2004002490, WO2004014361, WO2004041210, WO2004096982, WO2002050036, WO2004058144, WO2004087145, WO2003082835, WO2002026723, WO06002047, WO06014580, WO06002047, WO06014580, WO2006021448, WO06134378 and WO06137485. Further details for the preparation of compounds of formula (V) and (VII) are found in the examples.

Where D is O, Z¹ is CH and Z² is N, compounds of formula (V) can be prepared as follows:

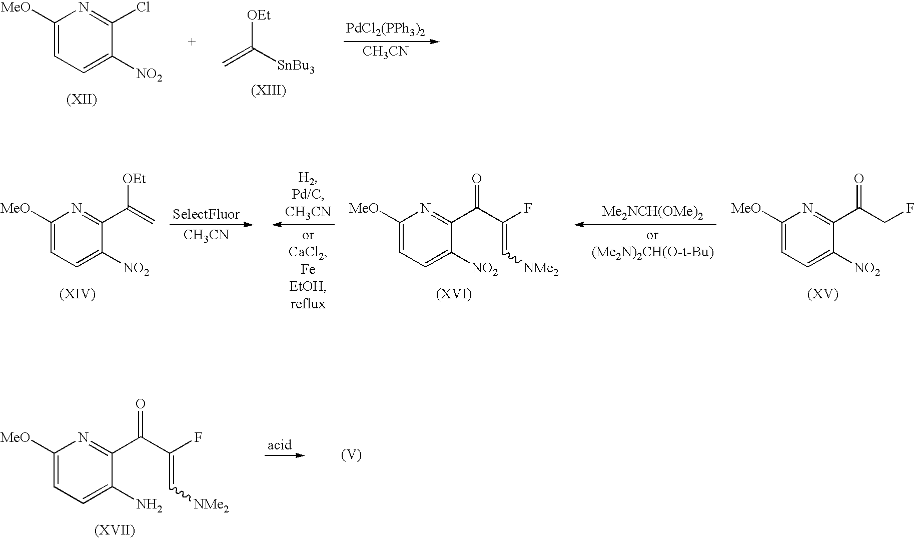

Enol-ethers (XIV) can be synthesised from aryl-halides (XII) via Stille coupling with a suitable alkoxyvinylstannane such as (XIII) (see Dhar et al, Org. Lett., 2002, 4, 2091) or via Heck coupling (see Xu et al, J. Mol. Catalysis. A: Chemical, 2002, 187, 189). Treatment of (XIV) with an electrophilic fluorinating agent such as SelectFluor® [1-(chloromethyl)-4-fluoro-1,4-diazoniabicyclo[2.2.2]octane bis(tetrafluoroborate] gives fluoroketone (XV). Conversion of ketone (XV) to enamino-ketone (XVI) can be accomplished by reagents such as Bredereck's or Emmons reagents amongst others, (for an example see Wasserman et al, J. Org. Chem. 1985, 50, 3573). Reduction of nitro ketone (XVI) using iron results in the corresponding amine (XVII), which on treatment with acid cyclises to give the hydroxynaphthyridine (V). Many other methods are known to reduce nitro groups (see for a review, Rylander P. N. Hydrogenation methods; Academic Press: NY:1985. For other methods see Smith, M. B.; March, J. M. Advanced Organic Chemistry, Wiley-Interscience, 2001, 1552).

Hydroxy derivatives (V) may be converted to bromo derivatives (VII) by treatment with a brominating agent such as PBr₃ in a suitable solvent such as DMF.

Where Z¹ and Z² are both C, bromoquinoline of formula (VII) may be prepared as follows:

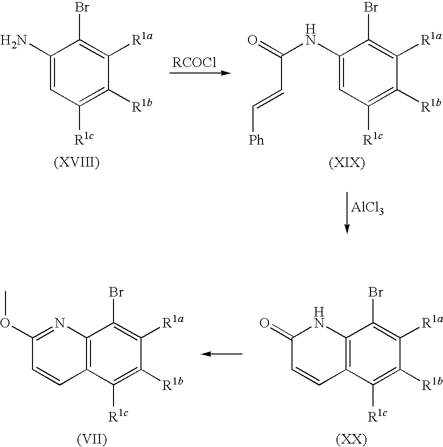

The aniline (XVIII) may be converted to the cinnamide (XIX), which can be cyclised with aluminium chloride (with loss of the phenyl moiety—See M. C. Elliot et al. J. Med. Chem. 47 (22), 5405-5417 (2004), S. R. Inglis et al. Synlett, 5, 898-900 (2004) or Cottet, F.; Marull, M.; Lefebvre, O.; Schlosser, M European Journal of Organic Chemistry (2003), 8, 1559) to give (XX). The quinolone (XX) is converted to (VII) by alkylation under conventional conditions, for example using diazomethane, trimethylsilyl diazomethane or iodomethane in the presence of base such as potassium carbonate.

Compounds of formula (III) HA-N(R²⁰)R^{2′} are known compounds or may be prepared analogously to known compounds, see for example WO2004/035569, WO2004/089947, WO02/08224, WO02/50061, WO02/56882, WO02/96907, WO2003087098, WO2003010138, WO2003064421, WO2003064431, WO2004002992, WO2004002490, WO2004014361, WO2004041210, WO2004096982, WO2002050036, WO2004058144, WO2004087145, WO2003082835, WO2002026723, WO06002047, WO06014580, WO06002047, WO06014580, WO2006021448, WO06134378, WO06137485, WO07016610, WO07081597, WO07071936, WO07115947, WO07118130, WO07122258, WO08006648, WO08003690 and WO08009700.

Further details for the preparation of compounds of formula (I) are found in the examples.

The antibacterial compounds according to the invention may be formulated for administration in any convenient way for use in human or veterinary medicine, by analogy with other antibacterials/antitubercular compounds.

The pharmaceutical compositions of the invention may be formulated for administration by any route and include those in a form adapted for oral, topical or parenteral use and may be used for the treatment of bacterial infection including tuberculosis in mammals including humans.

The composition may be in the form of tablets, capsules, powders, granules, lozenges, suppositories, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilizing the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1% by weight, preferably from 10-60% by weight, of the active material, depending on the method of administration. Where the compositions comprise dosage units, each unit will preferably contain from 50-1000 mg of the active ingredient. The dosage as employed for adult human treatment will preferably range from 100 to 3000 mg per day, for instance 1500 mg per day depending on the route and frequency of administration. Such a dosage corresponds to about 1.5 to about 30 mg/kg per day. Suitably the dosage is from 5 to 20 mg/kg per day.

The compound of formula (I) may be the sole therapeutic agent in the compositions of the invention or a combination with other antibacterials including antitubercular compounds. If the other antibacterial is a β-lactam then a β-lactamase inhibitor may also be employed.

Compounds of formula (I) may be used in the treatment of bacterial infections caused by a wide range of organisms including both Gram-negative and Gram-positive organisms, such as upper and/or lower respiratory tract infections, skin and soft tissue infections and/or urinary tract infections. Compounds of formula (I) may be also used in the treatment of tuberculosis caused by Mycobacterium tuberculosis. Some compounds of formula (I) may be active against more than one organism. This may be determined by the methods described herein.

The following examples illustrate the preparation of certain compounds of formula (I) and the activity of certain compounds of formula (I) against various bacterial organisms including Mycobacterium tuberculosis.

### Biological Activity

### Antimicrobial Activity Assay:

Whole-cell antimicrobial activity was determined by broth microdilution using the Clinical and Laboratory Standards Institute (CLSI) recommended procedure, Document M7-A7, “Methods for Dilution Susceptibility Tests for Bacteria that Grow Aerobically”. The compounds were tested in serial two-fold dilutions ranging from 0.016 to 16 mcg/mL.

Compounds were evaluated against Gram-positive organisms, including Staphylococcus aureus, Streptococcus pneumoniae, Streptococcus pyogenes, Enterococcus faecalis and Enterococcus faecium.

In addition, compounds were evaluated against Gram-negative organisms including Haemophilus influenzae, Moraxella catarrhalis, Escherichia coli, Pseudomonas aeruginosa, Proteus mirabilis, Legionella pneumophila, Enterobacter cloacae, Enterobacter aerogenes, Klebsiella pneumoniae and Stenotrophomonas maltophilia.

The L. pneumophila isolates were tested using a modified CLSI procedure for broth microdilution. For this assay, compounds were tested in serial doubling dilutions over a concentration range of 0.03 to 32 mcg/mL. An inoculum of each test isolate was prepared in buffered yeast broth and adjusted to a density equivalent to a 0.5 McFarland standard. After inoculation, the microtitre plates were incubated at 37° C. for 72 hours.

The minimum inhibitory concentration (MIC) was determined as the lowest concentration of compound that inhibited visible growth. A mirror reader was used to assist in determining the MIC endpoint.

Each of the listed Examples, as identified in the present application, were tested in at least one exemplified salt or free base form. Unless otherwise noted, the listed Examples had a MIC ≦2 μg/ml against a strain of at least one of the organisms listed above with the exception of Examples 88 and 97 which showed no activity against the listed organisms at ≦16 μg/ml. For at least one strain of every organism listed above, at least one Example had a MIC ≦2 μg/ml with the exception of strains of Proteus mirabilis for which at least one Example had a MIC ≦8 μg/ml and strains of Pseudomonas aeruginosa, for which at least one Example had a MIC ≦16 μg/ml.
Mycobacterium tuberculosis H37Rv Inhibition Assay

The measurement of the minimum inhibitory concentration (MIC) for each tested compound was performed in 96 wells flat-bottom, polystyrene microtiter plates. Ten two-fold drug dilutions in neat DMSO starting at 400 μM were performed. Five μl of these drug solutions were added to 95 μl of Middlebrook 7H9 medium. (Lines A-H, rows 1-10 of the plate layout). Isoniazid was used as a positive control, 8 two-fold dilution of Isoniazid starting at 160 μgml⁻¹ was prepared and 5 μl of this control curve was added to 95 μl of Middlebrook 7H9 (Difco catalogue Ref. 271310)+ADC medium (Becton Dickinson Catalogue Ref 211887). (Row 11, lines A-H). Five μl of neat DMSO were added to row 12 (growth and Blank controls).

The inoculum was standardised to approximately 1×10⁷ cfu/ml and diluted 1 in 100 in Middlebrook 7H9+ADC medium and 0.025% Tween 80 (Sigma P4780), to produce the final inoculum of H37Rv strain (ATCC25618). One hundred μl of this inoculum was added to the entire plate but G-12 and H-12 wells (Blank controls). All plates were placed in a sealed box to prevent drying out of the peripheral wells and they were incubated at 37° C. without shaking for six days. A resazurin solution was prepared by dissolving one tablet of resazurin (Resazurin Tablets for Milk Testing; Ref 330884Y VWR International Ltd) in 30 ml sterile PBS (phosphate buffered saline). 25 μl of this solution was added to each well. Fluorescence was measured (Spectramax M5 Molecular Devices, Excitation 530 nm, Emission 590 nm) after 48 hours to determine the MIC value.

Examples 2, 7, 10, 20, 22, 28, 30, 31, 37, 43, 48, 50, 52, 54, 56, 57, 67, 82 and 94-96 were tested in the Mycobacterium tuberculosis H37Rv inhibition assay. Examples 20, 28, 30, 37, 43, 48, 50, 52, 54, 67 and 82 showed an MIC value of 2.8 μg/ml or lower. Examples 28, 37, 43, 52, 54, 67 and 82 showed an MIC value of 1.0 μg/ml or lower.

### EXAMPLES AND EXPERIMENTAL

### General

### Abbreviations in the Examples:

RT=room temperature
S.T.P=standard temperature and pressure
ES=Electrospray mass spec.
LC-MS=Liquid chromatography mass spec.
HPLC=High Performance Liquid Chromatography (Rt refers to retention time) Certain reagents are also abbreviated herein. DCM refers to dichloromethane, DMSO refers to dimethylsulfoxide, DMF refers to N,N-dimethylformamide, TFA refers to trifluoroacetic acid, THF refers to tetrahydrofuran, TEA refers to triethylamine, Pd/C refers to palladium on carbon catalyst. BOC refers to t-butoxycarbonyl. Glycidyl nosylate refers to glycidyl-3-nitrobenzene sulfonate. NBS refers to N-bromosuccinnimide. IBX refers to the cyclic tautomer of 2-iodoxybenzoic acid. DMF-DMA refers to N,N′dimethylformamide dimethyl acetal. DME refers to 1,2,-dimethoxyethane. (S)-(−)-tol-BINAP refers to S-(−)-2,2′-bis(di-p-tolylphosphino)-1,1′-binaphthyl. MeCN refers to acetonitrile, Selectfluor™ (Air Products and Chemicals Ltd) is 1-chloromethyl-4-fluoro-1,4-diazoniabicyclo[2.2.2]octane bis(tetrafluoroborate)
Proton nuclear magnetic resonance (¹H NMR) spectra were recorded at 400 or 250 MHz, and chemical shifts are reported in parts per million (δ) downfield from the internal standard tetramethylsilane (TMS). Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz. CDCl₃ is deuteriochloroform and DMSO-d₆ is hexadeuteriodimethylsulfoxide. Mass spectra were obtained using electrospray (ES) ionization techniques. All temperatures are reported in degrees Celsius.
Chiralpak AD and AD-H columns comprise silica for preparative columns (Sum particle size AD-H, 21×250 mm; 20 um particle size AD, 101.6×250 mm) coated with Amylose tris (3,5-dimethylphenylcarbamate; 20 um particle size AS-H, 20×250 mm coated with amylose tris [(S)-alpha-methylbenzylcarbamate] (Chiral Technologies USA). Measured retention times are dependent on the precise conditions of the chromatographic procedures. Where quoted below in the Examples they are indicative of the order of elution.
SPE refers to a column of silica gel for Chromatography, manufactured by Isolute, UK
SCX Cartridge is an ion exchange column containing strong cation exchange resin (benzene sulfonic acid) supplied by Varian, USA.
Reactions involving metal hydrides including lithium hydride, lithium aluminium hydride, di-isobutylaluminium hydride, sodium hydride, sodium borohydride, are carried out under argon or other inert gas.
As will be understood by the skilled chemist, references to preparations carried out in a similar manner to, or by the general method of, other preparations, may encompass variations in routine parameters such as time, temperature, workup conditions, minor changes in reagent amounts etc.

### Example 1

### 3-({4-[(2,3-Dihydro[1,4]dioxino[2,3-c]pyridin-7-ylmethyl)amino]-1-piperidinyl}methyl)-10-fluoro-2,3-dihydro-1H,5H-pyrido[3,2,1-ij]quinolin-5-one dihydrochloride

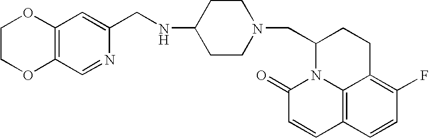

### (a) N-(2-Bromo-3-fluorophenyl)-3-phenyl-2-propenamide

To a solution of 2-bromo-3-fluoroaniline (5.29 g, 27.8 mmol) in acetone (12 ml) was added potassium carbonate (5.75 g, 41.8 mmol) followed by water (15 ml) and the stirred mixture cooled to 0° C. Cinnamoyl chloride (4.63 g, 27.8 mmol) was added portionwise over 15 min then the mixture stirred for a further 2 h. After this time the reaction mixture was poured onto ice/water and the resulting precipitate collected and dried to yield a white solid (8.1 g).

MS (ES+) m/z 320/322 (MH)⁺.

### (b) 8-Bromo-7-fluoro-2(1H)-quinolinone

To a stirred suspension of N-(2-bromo-3-fluorophenyl)-3-phenyl-2-propenamide (8.0 g, 25 mmol) in chlorobenzene (40 ml) was added aluminium chloride (20.0 g, 150 mmol) portionwise over about 5 min. The mixture was then heated to 120° C. for 1.5 h cooled to ˜50° C. and added slowly onto ice. A pink oil/solid separated out and the mixture was allowed to stand overnight. The mixture was extracted with ethyl acetate and the organics dried and concentrated to a reddish solid (4.9 g) which was largely the title compound. The solids were washed with hexane and dried to yield a pink solid (2.35 g).

MS (ES+) m/z 242/244 (MH)⁺.

### (c) 8-Bromo-7-fluoro-2-(methyloxy)quinoline

To a solution of 8-bromo-7-fluoro-2(1H)-quinolinone (2.0 g, 8.3 mmol) in DMF (30 ml) was added potassium carbonate (2.28 g, 16.6 mmol) followed by methyl iodide (1.0 ml, 9.9 mmol) and the mixture stirred for 3 h. The mixture was separated between ethyl acetate and water and the organics isolated, dried and concentrated to provide a dark solid (2.1 g).

MS (ES+) m/z 257 (MH)⁺.

### (d) Butyl (2E)-3-[7-fluoro-2-(methyloxy)-8-quinolinyl]-2-propenoate

8-Bromo-7-fluoro-2-(methyloxy)quinoline (5 g, 19.5 mmole) was treated with n-butyl acrylate (2.8 mL, 19.5 mmole) and dicyclohexylmethylamine (4.6 mL, 21.5 mmole) in 1,4-dioxane (50 mL) and de-gassed twice by placing under house vacuum then blanketing with argon. Tris(dibenzylidineacetone)dipalladium (0) (0.18 g, 0.2 mmole) (Strem Chemicals UK) and bis(tri-t-butylphosphine)palladium (0) (0.2 g, 0.4 mmole) (Strem Chemicals UK) were then added and the mixture heated at 70° C. for 18 h. The mixture was evaporated to dryness and the residue partitioned between water and ethyl acetate (2×150 mL). The combined organic layers were dried over sodium sulphate, filtered then evaporated to dryness. The residue was chromatographed on silica gel eluting with a gradient of 0-30% ethyl acetate in 40-60 petroleum ether. This gave the title compound as an oil (4.15 g).

¹H NMR (400 MHz, CDCl₃): δ 0.97 (3H, t, J 7.6 Hz), 1.39-1.51 (2H, m), 1.66-1.74 (2H, m), 4.12 (3H, s), 4.24 (2H, t, J 6.4 Hz), 6.89 (1H, d, J 9 Hz), 7.15-7.22 (2H, m), 7.66-7.71 (1H, m), 7.95 (1H, d, J 9 Hz), 8.68 (1H, d, J 16 Hz).

### (e) Butyl 3-[7-fluoro-2-(methyloxy)-8-quinolinyl]propanoate

Butyl (2E)-3-[7-fluoro-2-(methyloxy)-8-quinolinyl]-2-propenoate (3.8 g, 12.5 mmole) was dissolved in ethanol (200 mL) and hydrogenated at RT and one atmosphere of hydrogen over 5% palladium on carbon paste (5 g) for 6 h. The catalyst was then removed by filtration and the filtrate evaporated to dryness to give the title compound as an oil (3.15 g).

¹H NMR (250 MHz, CDCl₃): δ 0.89 (3H, t, J 7.6 Hz), 1.21-1.39 (2H, m), 1.47-1.62 (2H, m), 2.68-2.78 (2H, m), 3.44-3.53 (2H, m), 4.01-4.09 (5H, m), 6.84 (1H, d, J 9 Hz), 7.15 (1H, t, J 8 Hz), 7.49-7.59 (1H, m), 7.91 (1H, d, J 9 Hz).

### (f) 3-[7-Fluoro-2-(methyloxy)-8-quinolinyl]-1-propanol

A solution of butyl 3-[7-fluoro-2-(methyloxy)-8-quinolinyl]propanoate (3.15 g, 10 mmole) in THF (50 mL) was added to a solution of 1.0M lithium aluminium hydride in THF (11.4 mL, 11.4 mmole) in THF (100 mL) dropwise at 0-5° C. over 10 min. The mixture was allowed to warm to RT then stirred for 2 days. A mixture of water (6.4 mL) and 2.0M sodium hydroxide (3.8 mL) was added dropwise followed by diethyl ether (100 mL) and sodium sulphate. The mixture was then filtered and the solid washed with more diethyl ether (2×50 mL). The combined filtrates were evaporated to dryness to give the title compound as an oil (2.5 g).

¹H NMR (250 MHz, CDCl₃): δ 1.92-2.04 (2H, m), 3.28-3.38 (2H, m), 3.39-3.45 (2H, m), 6.83 (1H, d, J 9 Hz), 7.15 (1H, t, J 8 Hz), 7.51-7.59 (1H, m), 7.95 (1H, d, J 9 Hz).

### (g) 3-[7-Fluoro-2-(methyloxy)-8-quinolinyl]propanal

3-[7-Fluoro-2-(methyloxy)-8-quinolinyl]-1-propanol (2.5 g, 10 mmole) was dissolved in acetone (100 mL) and treated with IBX (D. B. Dess and J. C. Martin, J. Org. Chem. 1983, 48, 4155-4156) (4.2 g, 15 mmole) then stirred at RT for 18 h. The mixture was then heated at 40° C. for 6 h then left at RT for a further 18 h. The mixture was then filtered and the filtrate evaporated to dryness. Chromatography of the residue on silica gel eluting with 25% ethyl acetate/40-60 petroleum ether gave the title compound as an oil (2.4 g).

¹H NMR (250 MHz, CDCl₃): δ 2.79-2.89 (2H, m), 3.43-3.52 (2H, m), 4.04 (3H, s), 6.83 (1H, d, J 9 Hz), 7.15 (1H, t, J 8 Hz), 7.52-7.61 (1H, m), 7.92 (1H, d, J 9 Hz).

### (h) 7-Fluoro-2-(methyloxy)-8-[2-(2-oxiranyl)ethyl]quinoline

To a mixture of trimethylsulphoxonium iodide (2.72 g, 12 mmole) in DMF (50 mL) at 0-5° C. was added sodium hydride (60% dispersion in oil) (0.5 g, 12.5 mmole) and stirred for 30 min. A solution of 3-[7-fluoro-2-(methyloxy)-8-quinolinyl]propanal (2.4 g, 10 mmole) in DMF (20 mL) was then added and the mixture stirred for 3 h allowing to warm to RT. The mixture was then partitioned between water (100 mL) and ethyl acetate (150 mL) and the organic layer separated then washed with more water (2×100 mL). The mixture was then dried over sodium sulphate. Chromatography on silica gel eluting with 25% ethyl acetate/40-60 petroleum ether gave the title compound as an oil (1.04 g).

¹H NMR (250 MHz, CDCl₃): δ 1.83-2.05 (2H, m), 2.48-2.57 (1H, m), 2.65-2.73 (1H, m), 2.96-3.04 (1H, m), 3.28-3.38 (2H, m), 6.83 (1H, d, J 9 Hz), 7.15 (1H, t, J 8 Hz), 7.51-7.61 (1H, m), 7.92 (1H, d, J 9 Hz).

### (i) 10-Fluoro-3-(hydroxymethyl)-2,3-dihydro-1H,5H-pyrido[3,2,1-ij]quinolin-5-one

7-Fluoro-2-(methyloxy)-8-[2-(2-oxiranyl)ethyl]quinoline (5.76 g, 23 mmole) was dissolved in 1:1 DCM/THF (200 mL) and treated with ytterbium(III) triflate (1.45 g, 2.3 mmole) and stirred at RT for 3 h then heated at 40° C. for 1.5 h. More ytterbium(III) triflate (2.9 g, 4.6 mmole) was added and the mixture stirred at RT for 22 h. Saturated sodium bicarbonate solution (250 mL) was then added and the mixture stirred for 30 min. The mixture was then extracted with 10% methanol/DCM (3×200 mL) and the combined organic layers separated and dried over sodium sulphate, filtered then evaporated to dryness. Chromatography on silica gel eluting with a gradient of 0-10% methanol/DCM gave the title compound as a complex with ytterbium (4.0 g). (3.5 g). This material was dissolved in ethanol (150 mL) and treated with 2M sodium hydroxide solution (35 mL) and stirred at RT for 1 h. This mixture was concentrated to a small volume and water (100 mL) was added and the mixture extracted with 10% methanol/DCM (3×150 mL). The combined organic layers were dried over sodium sulphate filtered and evaporated to dryness to give the title compound as an off-white solid (2.2 g).

¹H NMR (250 MHz, CDCl₃): δ 1.79-1.97 (1H, m), 2.38-2.48 (1H, m), 2.65-2.83 (1H, m), 3.01-3.18 (2H, m), 3.72-3.91 (2H, m), 5.29-5.39 (1H, m), 6.75 (1H, d, J 9.5 Hz), 6.95 (1H, t, J 8 Hz), 7.35-7.45 (1H, m), 7.69 (1H, d, J 9.5 Hz).

### (j) 10-Fluoro-5-oxo-2,3-dihydro-1H,5H-pyrido[3,2,1-ij]quinoline-3-carbaldehyde

10-Fluoro-3-(hydroxymethyl)-2,3-dihydro-1H,5H-pyrido[3,2,1-ij]quinolin-5-one (2.2 g, 9 mmole) was dissolved in DCM (100 mL) and treated with Dess-Martin Periodinane (4.74 g, 11 mmole) then stirred at RT for 18 h. Diethyl ether (120 mL) and 1M sodium hydroxide solution (120 mL) were added and the mixture transferred to a separating funnel and extracted with 10% methanol/DCM (3×150 mL). The combined extracts were dried over sodium sulphate filtered then evaporated to dryness to give a mixture containing the title compound as a yellow oil (2.2 g).

MS (ES+), m/z 232 (MH⁺, 100%).

### (k) 1,1-Dimethylethyl {1-[(10-fluoro-5-oxo-2,3-dihydro-1H,5H-pyrido[3,2,1-ij]quinolin-3-yl)methyl]-4-piperidinyl}carbamate

A mixture of 10-fluoro-5-oxo-2,3-dihydro-1H,5H-pyrido[3,2,1-ij]quinoline-3-carbaldehyde (0.265 g, 1.2 mmole) and 1,1-dimethylethyl 4-piperidinylcarbamate (0.24 g, 1.2 mmole) in chloroform (10 mL), methanol (10 mL) and acetic acid (4 drops) was treated with (polystyrylmethyl)trimethylammonium cyanoborohydride 4.0 mmoles/gm (1.2 g, 4.8 mmoles) and stirred at RT for 18 h. More acetic acid (5 drops) and (polystyrylmethyl)trimethylammonium cyanoborohydride 4.0 mmoles/gm (1.2 g, 4.8 mmoles) were added and the mixture heated at 50° C. for 18 h. The mixture was then filtered and the filtrate evaporated to dryness. Chromatography of the residue on silica gel eluting with a gradient of 0-10% 2M ammonia/methanol in DCM gave a mixture containing the title compound as an oil (0.39 g).

MS (ES+), m/z 416 (MH⁺, 100%).

### (l) 3-[(4-Amino-1-piperidinyl)methyl]-10-fluoro-2,3-dihydro-1H,5H-pyrido[3,2,1-ij]quinolin-5-one

1,1-Dimethylethyl {1-[(10-fluoro-5-oxo-2,3-dihydro-1H,5H-pyrido[3,2,1-ij]quinolin-3-yl)methyl]-4-piperidinyl}carbamate (0.39 g, 0.9 mmole) in DCM (10 mL) was treated with TFA (10 mL), and stirred at RT for 2 h. The mixture was evaporated to dryness and the residue partitioned between saturated potassium carbonate solution and 10% methanol/DCM (3×30 mL). The combined organics were dried over sodium sulphate. Chromatography on silica gel eluting with a gradient of 0-20% 2M ammonia/methanol in DCM gave the title compound as a pale yellow oil (0.179 g).

MS (ES+), m/z 316 (MH⁺, 85%).

### (m) Title Compound

A mixture of 3-[(4-amino-1-piperidinyl)methyl]-10-fluoro-2,3-dihydro-1H,5H-pyrido[3,2,1-ij]quinolin-5-one (0.04 g, 0.13 mmole) and 2,3-dihydro[1,4]dioxino[2,3-c]pyridine-7-carbaldehyde (for a synthesis see WO2004058144, Example 2(c) or WO2003087098 Example 19(d) (0.021 g, 0.13 mmole) in chloroform (5 mL), methanol (5 mL) and acetic acid (5 drops) was treated with (polystyrylmethyl)trimethylammonium cyanoborohydride 4.0 mmoles/gm (0.27 g, 1.1 mmoles) and stirred at RT for 2.5 days. The mixture was then filtered and the resin washed with 1:1 DCM/methanol (2×20 mL) and the combined filtrate and washings evaporated to dryness. Chromatography on silica gel eluting with a gradient of 0-12% 2M ammonia/methanol in DCM gave the title compound as a free base (0.052 g).

¹H NMR (250 MHz, CDCl₃): δ 1.35-2.88 (14H, m), 2.93-3.21 (2H, m), 3.81 (2H, s), 4.22-4.38 (4H, m), 5.22-5.33 (1H, m), 6.61 (1H, d, J 9.5 Hz), 6.79 (1H, s), 6.89 (1H, t, J 8 Hz), 7.29-7.39 (1H, m), 7.59 (1H, d, J 9.5 Hz), 8.12 (1H, s).

MS (ES+), m/z 465 (MH⁺, 100%).

The free base in methanol was converted to the dihydrochloride salt by addition of 4.0M hydrogen chloride in 1,4-dioxane then evaporating to dryness to give a white solid.

### Example 2

### 10-Fluoro-3-({4-[([1,3]oxathiolo[5,4-c]pyridin-6-ylmethyl)amino]-1-piperidinyl}methyl)-2,3-dihydro-1H,5H-pyrido[3,2,1-ij]quinolin-5-one dihydrochloride salt

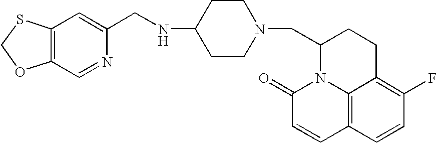

A mixture of 3-[(4-amino-1-piperidinyl)methyl]-10-fluoro-2,3-dihydro-1H,5H-pyrido[3,2,1-ij]quinolin-5-one (0.04 g, 0.13 mmole) and [1,3]oxathiolo[5,4-c]pyridine-6-carbaldehyde (for a synthesis see WO2004058144 Example 61) (0.021 g, 0.13 mmole) in chloroform (5 mL), methanol (5 mL) and acetic acid (5 drops) was treated with (polystyrylmethyl)trimethylammonium cyanoborohydride 4.0 mmoles/gm (0.27 g, 1.1 mmoles) and stirred at RT for 2.5 days. The mixture was then filtered and the resin washed with 1:1 DCM/methanol (2×20 mL) and the combined filtrate and washings evaporated to dryness. Chromatography on silica gel eluting with a gradient of 0-12% 2M ammonia/methanol in DCM gave the title compound as a free base (0.051 g).

¹H NMR (250 MHz, CDCl₃): δ 1.33-2.87 (14H, m), 2.93-3.21 (2H, m), 3.83 (2H, s), 5.19-5.31 (1H, m), 5.72 (2H, s), 6.61 (1H, d, J 9.5 Hz), 6.91 (1H, t, J 8 Hz), 7.20 (1H, s) 7.29-7.39 (1H, m), 7.59 (1H, d, J 9.5 Hz), 8.00 (1H, s).

MS (ES+), m/z 467 (MH⁺, 100%).

The free base in methanol was converted to the dihydrochloride salt by addition of 4.0M hydrogen chloride in 1,4-dioxane then evaporating to dryness to give a white solid.

### Example 3

### 3-({4-[(6,7-Dihydro[1,4]dioxino[2,3-c]pyridazin-3-ylmethyl)amino]-1-piperidinyl}methyl)-10-fluoro-2,3-dihydro-1H,5H-pyrido[3,2,1-ij]quinolin-5-one dihydrochloride salt

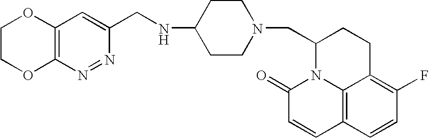

### (a) 3,4,6-Trichloropyridazine

This was prepared by a slight variation on the method of Kasnar et al, Nucleosides & Nucleotides (1994), 13(1-3), 459-79.

Hydrazine sulphate salt (51 g) was suspended in water (250 ml), heated to reflux and bromomaleic anhydride (90.38 g) was added dropwise. The mixture was heated at reflux for 4 hours then cooled to room temperature. The reaction was repeated with 29 g hydrazine sulphate, 53 g bromomaleic anhydride and 130 ml water. The precipitates were collected by filtration, washed with water and acetone and dried as a combined batch in vacuo to afford 4-bromo-1,2-dihydro-3,6-pyridazinedione as a white solid (113 g).

The solid in two batches was treated with phosphorus oxychloride (2×200 ml) and heated to reflux for 3.5 hours. The mixture was cooled, evaporated and azeotroped with toluene. The residue was partitioned between dichloromethane and saturated aqueous sodium bicarbonate solution and extracted with DCM twice more. The organic extracts were dried and evaporated. This residue was re-dissolved in dichloromethane, and chromatographed on silica gel (300 g) (DCM as eluent) to give a white solid (101.5 g, 87%).

(LC/MS analysis showed ca 20-30% impurity, isomers of bromo-dichloropyridazine).

MS (+ve ion electrospray) m/z 184/185/186 (MH+), trichloropyridazine.

MS (+ve ion electrospray) m/z 228/229/231 (MH+), bromo-dichloropyridazine.

### (b) 2-[(3,6-Dichloro-4-pyridazinyl)oxy]ethanol

A solution of ethylene glycol (55 ml) in tetrahydrofuran (200 ml) was treated at around 0° C. (ice bath cooling) with sodium hydride (60% dispersion in oil, 5.9 g) over 40 minutes. After the addition was complete, 3,4,6-trichloropyridazine (27 g) containing isomers of bromo-dichloropyridazine as impurity was added portionwise and washed in with more dry THF (50 ml) and the mixture was stirred at 0° C. for 1 hour and then at room temperature overnight. The mixture was concentrated (to 1/3 volume) then diluted with aqueous sodium bicarbonate solution and extracted with chloroform (5×) and ethyl acetate (3×). The combined organic extracts were washed with water, dried over sodium sulphate and evaporated and the solids filtered off and washed with CHCl3 (×3) and dried in a vacuum oven overnight at 40° C. affording a white solid (25.5 g, 83%), containing some bromo-derivative (10-15%).

MS (+ve ion electrospray) m/z 209/211 (MH+).

MS (+ve ion electrospray) m/z 255/7 (MH+), bromo-derivative.

### (c) 3-Chloro-6,7-dihydro[1,4]dioxino[2,3-c]pyridazine

A solution of 2-[(3,6-dichloro-4-pyridazinyl)oxy]ethanol containing some bromo-derivative (15.46 g; 0.0703 mol) in dry 1,4-dioxane (1.2 L) was treated with lithium hydride (2.3 g; 0.28 mol) in portions and stirred at room temperature for 1 hour under argon, then heated at 110° C. overnight. The reaction mixture was quenched with wet 1,4-dioxane, then iced-water. The solution was evaporated to half volume, taken to pH 8 with 5M hydrochloric acid and evaporated to dryness. Water was added and the residue was extracted 5× with chloroform, dried (sodium sulphate) and evaporated to afford a white solid (12.4 g, ca. 77%) (containing ca. 15% of a bromo species).

MS (+ve ion electrospray) m/z 173/5 (Cl MH+); 217/9 (Br MH+)

### (d) 3-Ethenyl-6,7-dihydro[1,4]dioxino[2,3-c]pyridazine

A solution of 3-chloro-6,7-dihydro[1,4]dioxino[2,3-c]pyridazine (13.6 g, 0.079 mol) containing ca. 15% of a bromo species in dimethoxyethane (400 ml) was degassed under argon for 10 min then tetrakis(triphenylphosphine)palladium (0) (2 g), potassium carbonate (10.33 g), 2,4,6-trivinylcyclotriboroxane pyridine complex (11.32 g) and water (55 ml) were added. The mixture was heated at 95° C. for 48 hours and cooled and evaporated to dryness. The mixture was treated with aqueous sodium bicarbonate solution and extracted (5×) with DCM. Extracts were dried (sodium sulphate), evaporated and the residue chromatographed on silica gel (500 g), eluting with 0-100% ethyl acetate-hexane, affording the product (6.43 g, 50%); [also some impure fractions (1.8 g)].

MS (+ve ion electrospray) m/z 165 (MH+).

### (e) 6,7-Dihydro[1,4]dioxino[2,3-c]pyridazine-3-carbaldehyde

A solution of 3-ethenyl-6,7-dihydro[1,4]dioxino[2,3-c]pyridazine (11.58 g) in 1,4-dioxane/water (600 ml/180 ml), cooled in ice, was treated with an aqueous solution of osmium tetroxide (4% w/v, 25 ml) and sodium periodate (43 g). This mixture was allowed to warm to room temperature and after 7 hours under stirring the mixture was evaporated to dryness and azeotroped with 1,4-dioxane. Silica gel, 1,4-dioxane and chloroform were added and the mixture was evaporated to dryness overnight, then added to a silica column (400 g) and chromatographed, eluting with chloroform then 0-100% ethyl acetate in hexane, to afford a white solid (7.55 g, 64%).

MS (+ve ion electrospray) m/z 167 (MH+).

### (f) Title Compound

A mixture of 3-[(4-amino-1-piperidinyl)methyl]-10-fluoro-2,3-dihydro-1H,5H-pyrido[3,2,1-ij]quinolin-5-one (0.04 g, 0.13 mmole) and 6,7-dihydro[1,4]dioxino[2,3-c]pyridazine-3-carbaldehyde (0.021 g, 0.13 mmole) in chloroform (5 mL), methanol (5 mL) and acetic acid (5 drops) was treated with (polystyrylmethyl)trimethylammonium cyanoborohydride 4.0 mmoles/gm (0.27 g, 1.1 mmoles) and stirred at RT for 2.5 days. The mixture was then filtered and the resin washed with 1:1 DCM/methanol (2×20 mL) and the combined filtrate and washings evaporated to dryness. Chromatography on silica gel eluting with a gradient of 0-12% 2M ammonia/methanol in DCM gave the title compound as a free base (0.045 g).

¹H NMR (250 MHz, CDCl₃): δ 1.31-1.54 (2H, m), 1.64-2.83 (12H, m), 2.93-3.19 (2H, m), 3.98 (2H, s), 4.31-4.39 (2H, m), 4.44-4.52 (2H, m), 5.21-5.32 (1H, m), 5.72 (2H, s), 6.61 (1H, d, J 9.5 Hz), 6.93 (1H, t, J 8 Hz), 7.02 (1H, s) 7.31-7.40 (1H, m), 7.60 (1H, d, J 9.5 Hz).

MS (ES+), m/z 466 (MH⁺, 60%).

The free base in methanol was converted to the dihydrochloride salt by addition of 4.0M hydrogen chloride in 1,4-dioxane then evaporating to dryness to give a white solid.

### Example 4

### 3-({4-[(6,7-Dihydro[1,4]oxathiino[2,3-c]pyridazin-3-ylmethyl)amino]-1-piperidinyl}methyl)-10-fluoro-2,3-dihydro-1H,5H-pyrido[3,2,1-ij]quinolin-5-one dihydrochloride salt

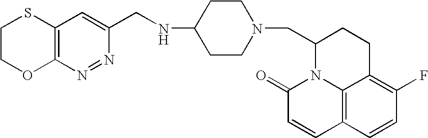

### (a) 2-[(3,6-Dichloro-4-pyridazinyl)thio]ethanol

A solution of 3,4,6-trichloropyridazine (25 g) in tetrahydrofuran (200 ml) and triethylamine (19 ml) was treated at 0° C. (ice bath cooling) with 2-mercaptoethanol (8.33 ml) over 5 minutes. After the addition was complete, the mixture was stirred at room temperature for 72 hours. The mixture was stirred with aqueous sodium bicarbonate solution and dichloromethane and the solid was collected, washed with water, ether and pentane and dried in vacuo, giving (22.9 g). The combined aqueous and organic fraction was evaporated to half volume giving further solid, which was washed and dried as above (5.0 g). The total yield of solid (27.9 g; 91%) contained some bromo-analogue (5-10%) by NMR.

### (b) 3-Chloro-6,7-dihydro[1,4]oxathiino[2,3-c]pyridazine

A solution of 2-[(3,6-dichloro-4-pyridazinyl)thio]ethanol (13 g) (previously dried at 50° C. in vacuo) in dry 1,4-dioxane (250 ml) was treated with lithium hydride (3 g) in portions and heated at 105-110° C. for 24 hours. The reaction mixture was cooled and quenched with iced-water. The solution was taken to pH 10-11 with 5M hydrochloric acid and evaporated. Water was added and the mixture was extracted 4× with dichloromethane, dried (sodium sulphate), evaporated, and chromatographed on silica gel, eluting with 0-100% ethyl acetate-hexane, to afford a white solid (1.61 g) (containing ca. 10% of the bromo species).

MS (+ve ion electrospray) m/z 189/91 (Cl MH+); 233/5 (Br MH+).

δH (CDCl3, 400 MHz) 3.23 (2H, m), 4.67 (2H, m), 7.26 (1H, s) (for major chloro-compound).

### (c) 3-Ethenyl-6,7-dihydro[1,4]oxathiino[2,3-c]pyridazine

A solution of 3-chloro-6,7-dihydro[1,4]oxathiino[2,3-c]pyridazine (1.0 g) in dimethoxyethane (25 ml) was degassed under argon then tetrakis(triphenylphosphine)palladium (0) (135 mg), potassium carbonate (0.695 g), 2,4,6-trivinylcyclotriboroxane pyridine complex (0.8 g) and water (3.7 ml) were added. The mixture was heated at 105° C., overnight. More 2,4,6-trivinylcyclotriboroxane pyridine complex (0.4 g) and tetrakis(triphenylphosphine)palladium (0) (30 mg) were added and heating was continued for 24 hours. The mixture was cooled, treated with aqueous sodium bicarbonate solution, extracted (4×) with DCM, dried (sodium sulphate), evaporated and chromatographed on silica gel (70 g), eluting with 0-100% ethyl acetate-hexane, affording a solid (0.56 g) (87% pure by LC-MS).

MS (+ve ion electrospray) m/z 181 (MH+).

### (d) 6,7-Dihydro[1,4]oxathiino[2,3-c]pyridazine-3-carbaldehyde

A solution of 3-ethenyl-6,7-dihydro[1,4]oxathiino[2,3-c]pyridazine (320 mg) in 1,4-dioxane/water (20 ml/5 ml) was treated with an aqueous solution of osmium tetroxide (4% w/v, 2 ml) and sodium periodate (1.08 g), initially stirred in an ice-bath, then allowed to warm to room temperature. After 2.5 hours the mixture was evaporated to dryness and dissolved in 1,4-dioxane and chloroform. Silica gel was added and the mixture was evaporated to dryness, added to a silica column (50 g) and chromatographed, eluting with 0-100% ethyl acetate in hexane, to afford a white solid (116 mg, 36%).

MS (+ve ion electrospray) m/z 183 (MH+).

### (e) Title Compound

A mixture of 3-[(4-amino-1-piperidinyl)methyl]-10-fluoro-2,3-dihydro-1H,5H-pyrido[3,2,1-ij]quinolin-5-one (0.04 g, 0.13 mmole) and 6,7-dihydro[1,4]oxathiino[2,3-c]pyridazine-3-carbaldehyde (0.023 g, 0.13 mmole) in chloroform (5 mL), methanol (5 mL) and acetic acid (5 drops) was treated with (polystyrylmethyl)trimethylammonium cyanoborohydride 4.0 mmoles/gm (0.27 g, 1.1 mmoles) and stirred at RT for 2.5 days. The mixture was then filtered and the resin washed with 1:1 DCM/methanol (2×20 mL) and the combined filtrate and washings evaporated to dryness. Chromatography on silica gel eluting with a gradient of 0-12% 2M ammonia/methanol in DCM gave the title compound as a free base (0.033 g).

¹H NMR (400 MHz, CDCl₃) δ 1.35-1.49 (2H, m), 1.69-1.79 (1H, m), 1.81-1.95 (2H, m), 2.02-2.29 (3H, m), 2.33-2.41 (2H, m), 2.45-2.63 (2H, m), 2.71-2.87 (2H, m), 2.97-3.05 (1H, m), 3.11-3.19 (1H, m), 3.20-3.25 (2H, m), 3.95 (2H, s), 4.61-4.66 (2H, m), 5.21-5.32 (1H, m), 6.61 (1H, d, J 9.5 Hz), 6.93 (1H, t, J 8 Hz), 7.29 (1H, s) 7.30-7.39 (1H, m), 7.60 (1H, d, J 9.5 Hz).

MS (ES+), m/z 482 (MH⁺, 70%).

The free base in methanol was converted to the dihydrochloride salt by addition of 4.0M hydrogen chloride in 1,4-dioxane then evaporating to dryness to give a white solid.

### Example 5

### 3-({4-[(6,7-Dihydro[1,4]dioxino[2,3-c]pyridazin-3-ylmethyl)amino]-1-piperidinyl}methyl)-10-fluoro-2,3-dihydro-1H,5H-pyrido[3,2,1-ij]quinolin-5-one Enantiomer 1 monobenzoate salt and

### Example 6

### 3-({4-[(6,7-Dihydro[1,4]dioxino[2,3-e]pyridazin-3-ylmethyl)amino]-1-piperidinyl}methyl)-10-fluoro-2,3-dihydro-1H,5H-pyrido[3,2,1-ij]quinolin-5-one Enantiomer 2 monobenzoate salt

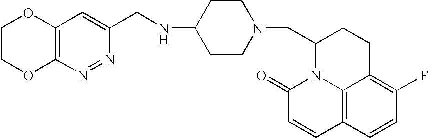

3-({4-[(6,7-Dihydro[1,4]dioxino[2,3-c]pyridazin-3-ylmethyl)amino]-1-piperidinyl}methyl)-10-fluoro-2,3-dihydro-1H,5H-pyrido[3,2,1-ij]quinolin-5-one (1.93 g) was resolved into its separate enantiomers using preparative HPLC (multiple injections) on a Chiralpac AS-H column (20×250 mm) and 0.1% isopropylamine in methanol mobile phase. This gave after evaporation of eluents enantiomer 1 as a pale yellow foam (0.83 g) with a retention time of 10.8 mins and enantiomer 2 as a light coloured foam (0.84 g) with a retention time of 14 mins. These were then converted into their monobenzoate salts to give the title compounds.

### Example 7

### (6R)-3-Bromo-6-({4-[(2,3-dihydro[1,4]dioxino[2,3-e]pyridin-7-ylmethyl)amino]-1-piperidinyl}methyl)-5,6-dihydro-8H-[1,4]oxazino[2,3,4-de]-1,5-naphthyridin-8-one dihydrochloride

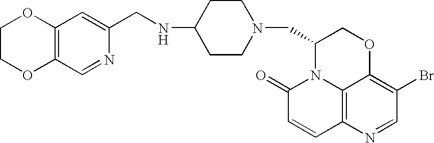

### (a) 3-Bromo-6-methoxy-1,5-naphthyridin-4-ol

6-Methoxy-1,5-naphthyridin-4-ol (for method of preparation see GB patent 1147760, 1965) (17.6 g, 10 mmol) was dissolved in glacial acetic acid (500 ml) and treated with N-bromosuccinimide (17.8 g, 10 mmol) over a period of 10 min. A yellow precipitate was observed, and the reaction mixture stirred at room temperature for a further 3 h. The mixture was concentrated under reduced pressure to a volume of 20 ml. This mixture was neutralised with half-saturated aqueous sodium hydrogen carbonate solution (strong effervescence). The resultant precipitate was collected by filtration, washed with water, and dried in vacuo at room temperature for 18 h, followed by 4 h at 50° C. The product was obtained as a buff-coloured solid (23.02 g, 90%).

MS (ES+), m/z 257, 259 (MH⁺, 100%).

### (b) 7-Bromo-2-methoxy-8-{[(2S)-2-oxiranylmethyl]oxy}-1,5-naphthyridine

3-Bromo-6-methoxy-1,5-naphthyridin-4-ol (7.68 g, 30 mmol) was dissolved in dry DMF (200 ml) and treated with potassium carbonate (4.14 g) followed by S-(+)-glycidyl nosylate (9.3 g, 30 mmol), and the mixture stirred at room temperature overnight. The solvent was evaporated under reduced pressure and the residue partitioned between water (150 ml) and ethyl acetate (150 ml). The aqueous phase was extracted with ethyl acetate (2×150 ml) and the combined aqueous phases dried (MgSO4) and evaporated. The residue was chromatographed on silica gel eluting with a gradient of 0-50% 2M ethyl acetate in 40-60 petroleum ether to give the title compound as a white solid (2.92 g, 32%).

MS (ES+), m/z 311, 313 (MH⁺, 100%).

### (c) (6R)-3-Bromo-6-(hydroxymethyl)-5,6-dihydro-8H-[1,4]oxazino[2,3,4-de]-1,5-naphthyridin-8-one

7-Bromo-2-methoxy-8-{[(2S)-2-oxiranylmethyl]oxy}-1,5-naphthyridine (2.94 g, 9.5 mmol) was dissolved in dichloromethane (75 ml) and THF (75 ml). The solution was treated with ytterbium (III) trifluoromethanesulphonate (5.78 g, 9.3 mmol) and the mixture stirred at room temperature under argon overnight. Saturated sodium hydrogen carbonate solution (50 ml) was added and the mixture stirred for 30 min, then concentrated under reduced pressure. The residue was partitioned between dichloromethane (100 ml) and water (100 ml) and the aqueous phase extracted with dichloromethane (3×100 ml). The combined organic extracts were dried (MgSO4) and concentrated to low volume (30 ml). A white precipitate formed which was collected by filtration and dried in vacuo to give the desired product as a white solid (1.65 g, 58%). The filtrate was applied to a column of silica gel and eluted with 0-10% methanol in dichloromethane to afford a further crop of the desired product (0.61 g, 22%).

MS (ES+), m/z 297, 299 (MH⁺, 100%). The material was crystallised from hot methanol. X-ray crystallography confirmed the stereocentre to have the (R) configuration.

### (d) [(6S)-3-Bromo-8-oxo-5,6-dihydro-8H-[1,4]oxazino[2,3,4-de]-1,5-naphthyridin-6-yl]methyl methanesulfonate

(6R)-3-Bromo-6-(hydroxymethyl)-5,6-dihydro-8H-[1,4]oxazino[2,3,4-de]-1,5-naphthyridin-8-one (545 mg, 1.83 mmol) was suspended in dichloromethane (25 ml), treated with triethylamine (0.32 ml, 2.2 mmol) and cooled in an ice-water bath. Methanesulfonyl chloride (0.17 ml, 2.2 mmol) was added and the cooling bath removed. The reaction mixture was stirred at room temperature for 1 h then diluted with dichloromethane and washed with water, saturated aqueous sodium hydrogen carbonate solution and brine. A white precipitate which did not dissolve in either layer was collected by filtration, and dried in vacuo to give the desired mesylate as a white solid (265 mg, 38%).

MS (ES+), m/z 375, 377 (MH⁺, 100%). A further crop was obtained by drying (MgSO4) and evaporating the organic phase.

### (e) 1,1-dimethylethyl (1-{[(6R)-3-bromo-8-oxo-5,6-dihydro-8H-[1,4]oxazino[2,3,4-de]-1,5-naphthyridin-6-yl]methyl}-4-piperidinyl)carbamate

[(6S)-3-bromo-8-oxo-5,6-dihydro-8H-[1,4]oxazino[2,3,4-de]-1,5-naphthyridin-6-yl]methyl methanesulfonate (232 mg, 0.62 mmol) was dissolved in acetonitrile (7 ml) and treated with dry pyridine (0.06 ml, 2.0 equiv) and 1,1-dimethylethyl 4-piperidinylcarbamate (260 mg, 2.0 equiv). The mixture was heated to 70° C. for 18 h. The mixture was cooled, diluted with ethyl acetate (20 ml), washed with saturated aqueous sodium hydrogen carbonate solution (10 ml), water (10 ml) and brine (10 ml) dried (MgSO4) and evaporated. The residue was chromatographed on silica gel eluting with 0-7% 2M methanolic ammonia in dichloromethane. Product-containing fractions were combined and evaporated to a white solid (134 mg, 45%).

MS (ES+), m/z 479, 481 (MH⁺, 100%).

### (f) Title Compound

1,1-dimethylethyl (1-{[(6R)-3-bromo-8-oxo-5,6-dihydro-8H-[1,4]oxazino[2,3,4-de]-1,5-naphthyridin-6-yl]methyl}-4-piperidinyl)carbamate (134 mg, 0.28 mmol) was dissolved in dichloromethane (5 ml) and treated with a solution of 4M HCl in 1,4-dioxane (1.0 ml, 4.0 mmol). The reaction mixture was stirred at room temperature for 30 mins then evaporated under reduced pressure to give a white solid. (147 mg, assumed to be the hydrated di-hydrochloride). This was dissolved in methanol (5 mL), and acetic acid (0.05 ml) and treated with 2,3-dihydro[1,4]dioxino[2,3-c]pyridine-7-carbaldehyde (for a synthesis see WO2004058144, Example 2(c) or WO2003087098 Example 19(d)) (0.046 g, 0.28 mmole). After stirring at room temperature for 40 min, (polystyrylmethyl)trimethylammonium cyanoborohydride 4.0 mmoles/gm (0.3 g, 1.2 mmol) was added and the mixture stirred at RT overnight. The mixture was then filtered and the filtrate evaporated to dryness. Chromatography on silica gel eluting with a gradient of 0-15% 2M ammonia/methanol in DCM gave the title compound as a free base as a colourless oil (100 mg, 67%).

¹H NMR (400 MHz, CDCl₃) δ 1.35-1.49 (2H, m), 1.79-1.95 (2H, m), 2.13-2.20 (1H, m), 2.28 (1H, dt, J 2.4, 11.2 Hz), 2.40 (1H, dd, J 2.8, 12.4 Hz), 2.45-2.53 (2H, m), 2.85 (1H, d, J 11.2 Hz), 3.15 (1H, d, J 11.6 Hz), 3.79 (2H, s), 4.16 (1H, d, J 10.8 Hz), 4.26-4.28 (2H, m) 4.31-4.34 (2H, m), 4.90-4.96 (1H, m), 5.10 (1H, dd, J 10.8 Hz), 6.81 (1H, s), 6.91 (1H, d, J 10.0 Hz), 7.86 (1H, d, J 10.0 Hz), 8.10 (1H, s), 8.50 (1H, s).

MS (ES+), m/z 530, 532 (MH⁺, 60%).

The free base of the title compound (100 mg, 0.19 mmol) was dissolved in methanol and treayed with 1M HCl in diethyl ether (0.8 ml, 0.8 mmol). After 10 min that resulting mixture was evaporated to dryness to afford the di-HCl salt as a yellow solid.

MS (ES+), m/z 530, 532 (MH⁺, 40%).

### Example 8

### (6R)-6-({4-[(2,3-Dihydro[1,4]dioxino[2,3-c]pyridin-7-ylmethyl)amino]-1-piperidinyl}methyl)-3-fluoro-5,6-dihydro-8H-[1,4]oxazino[2,3,4-de]-1,5-naphthyridin-8-one

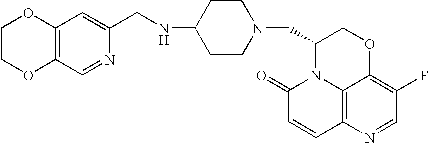

### (a) 2-[1-(Ethyloxy)ethenyl]-6-(methyloxy)-3-nitropyridine

Under N₂ was charged 2-chloro-6-methoxy-3-nitropyridine (600 g, 3.18 mol), MeCN (4200 ml, 7 vol) and PdCl₂(PPh₃)₂ (33.5 g, 477 mmol, 0.015 eq.). The yellow coloured suspension was heated to 65° C. and (1-ethoxyvinyl)-tributyl-stannane (1182 ml, 1264 g, 3.50 mol, 1.1 eq.) was added dropwise over 2 hours, maintaining the temperature at ˜65° C. by the rate of addition. The resulting brown coloured suspension was left to stir at 65° C. for 4 hours then left to cool to room temperature, with stirring, overnight. The reaction mixture was quenched with 10% KF aq. solution (3600 ml, 6 vol) with vigorous stirring and left to stir for 1 hour. The resulting solid was removed by vacuum filtration and washed with MeCN (7×1000 ml). The layers were separated and the organic layer was evaporated to 5 volumes (3000 ml). This was filtered through glass microfibre filter paper and the small amount of brown solid removed was washed with MeCN (1800 ml, 3 vol). EtOAc (3600 ml, 6 vol) was added and the volume reduced to 3 volumes (1800 ml). EtOAc (3600 ml) was added and the volume reduced to 3 volumes (1800 ml). Cyclohexane (3600 ml, 6 vol) was added and the volume reduced to 5 volumes (3000 ml). Cyclohexane (2400 ml, 4 vol) and silica gel (600 g, 1 wt) were added and allowed to stir at r.t. for 1.5 h. The solid was removed by vacuum filtration and washed with EtOAc:cyclohexane, 1:8 (4200 ml, 7 vol). The filtrate was reduced to 3 volumes (1800 ml). Cyclohexane (2400 ml, 4 vol) was added and the volume reduced to 3 volumes (1800 ml). Cyclohexane (3600 ml, 6 vol) and EtOAc (600 ml, 1 vol) and silica gel (600 g, 1 wt) were added and left to stir for 1.5 h. The solid was removed by vacuum filtration and washed with EtOAc:cyclohexane, 1:8 (4200 ml, 7 vol). The solvents were evaporated to dryness. MeCN (2000 ml) was added and evaporated to give the title compound as an orange coloured oil (93% yield)

### (b) 2-Fluoro-1-[6-(methyloxy)-3-nitro-2-pyridinyl]ethanone

Under N₂ was charged Selectfluor (1286.4 g, 3.63 mol), MeCN (2060 ml) and water (820 ml). A solution of 2-[1-(Ethyloxy)ethenyl]-6-(methyloxy)-3-nitropyridine (708.0 g, 3.16 mol) in MeCN (1416 ml) was added dropwise, to the white suspension, over 1.5 h, maintaining the temperature <15° C. using an ice/water bath. The resulting yellow coloured solution was left to stir at r.t. overnight. TLC (EtOAc:hexane, 1:4) indicated all the starting material had been consumed. The reaction mixture was quenched with sat. aq. NaHCO₃ (2140 ml) and left to stir for 30 minutes. The volume was reduced by rotary evaporation to 3250 ml. To the resulting yellow suspension was added EtOAc (4400 ml) and water (720 ml) and allowed to stir for 15 min. The layers were separated and the aqueous extracted with EtOAc (2×1000 ml). The organic layers were combined and washed with water (1000 ml) and sat. brine (1000 ml). The organic layer was dried over MgSO₄ (400 g), filtered and evaporated. MeCN (1000 ml) was added and evaporated to give the title compound as an orange oil which slowly solidified on standing. (97.2%)

### (c) 3-(Dimethylamino)-2-fluoro-1-[6-(methyloxy)-3-nitro-2-pyridinyl]-2-propen-1-one

To a solution of 2-fluoro-1-[6-(methyloxy)-3-nitro-2-pyridinyl]ethanone (657.0 g, 3.07 mol) in toluene (2700 ml) was added N,N-dimethylformamide dimethylacetal (1550 ml). The reaction mixture was heated to 50° C. and left to stir for 4 hours, under N₂. Product precipitated out after ˜1 h. Cyclohexane (2000 ml) was added and the reaction mixture left to cool slowly over 1 h, then to <5° C. using an ice/water bath. The solid was collected by vacuum filtration and washed with EtOAc:cyclohexane, 1:1 (3×1000 ml). The title compound was obtained as a yellow solid which was dried in the oven, under vacuum at 40° C. overnight. (yield 69.8%)

### (d) 3-Fluoro-6-(methyloxy)-1,5-naphthyridin-4-ol (Onyx EW412, EW434)

5% Pd/C, 58.2% water wet (274.4 g, 114.7 g active input, 10 wt %) was slurried in DMF (1000 ml) and charged to the 20 L bomb which had been pre-heated to 40° C. 3-(Dimethylamino)-2-fluoro-1-[6-(methyloxy)-3-nitro-2-pyridinyl]-2-propen-1-one (1146.7 g, 4.26 mol), which was dissolved in DMF (10500 ml) by warming to 40° C., was charged and the reaction mixture was stirred under H₂ at 15 psi for 3 h, maintaining the temperature between 45-50° C. HPLC indicated complete consumption of the starting material. The reaction mixture was warmed to 60° C. DMF (1800 ml) was warmed to 50° C. and charged to the pressurised filter. The hot reaction mixture was nitrogen transferred through the pressurised filter, at 15 psi, to remove the catalyst. The vessel was rinsed out with hot DMF (2×1500 ml), bringing the total volume of DMF to 16 volumes. The product, 1-[3-amino-6-(methyloxy)-2-pyridinyl]-3-(dimethylamino)-2-fluoro-2-propen-1-one was not isolated and used directly in the next stage.

(2Z)-1-[3-amino-6-(methyloxy)-2-pyridinyl]-3-(dimethylamino)-2-fluoro-2-propen-1-one in DMF (5500 ml, contains 264.6 g amine, 1.10 mol) was cooled to 5° C. using an ice/water bath. 6N HCl (184 ml, 1.10 mol, 1 eq.) was added dropwise to the reaction mixture over 30 minutes, slowly exothermic, temperature maintained <10° C. The reactions were allowed to warm to room temperature and left to stir overnight. The volume of each batch was reduced to 2000 ml by rotary evaporation at 50° C. The yellow suspension was charged back into the flange flask and cooled to 10° C. using an ice/water bath. Water (4000 ml) was added slowly over 30 minutes, temperature maintained <15° C. The reaction mixture was stirred vigorously for 1 h. The solid was collected by vacuum filtration and washed with water (3000 ml) then EtOAc:cyclohexane, 1:1 (3×2000 ml). The title compound was obtained as a pale brown solid was dried in the oven, under vacuum at 50° C. for 4 days. (76.9% yield).

### (e) 7-Fluoro-2-(methyloxy)-8-{[(2S)-2-oxiranylmethyl]oxy}-1,5-naphthyridine

3-Fluoro-6-(methyloxy)-1,5-naphthyridin-4-ol (5.82 g, 30 mmol) was reacted with S-(+) glycidyl nosylate (9.3 g, 30 mmol) under the general conditions described for Example 7 (b). After work-up and chromatography under the same general conditions, the desired compound was obtained as a white solid (4.58 g, 65%).

MS (ES+), m/z 251 (MH⁺, 100%).

### (f) (6R)-3-Fluoro-6-(hydroxymethyl)-5,6-dihydro-8H-[1,4]oxazino[2,3,4-de]-1,5-naphthyridin-8-one

7-Fluoro-2-(methyloxy)-8-{[(2S)-2-oxiranylmethyl]oxy}-1,5-naphthyridine (495 mg, 1.98 mmol) was treated with ytterbium (III) trifluoromethanesulphonate (1.2 g, 1.93 mmol) under the general conditions described for Example 7 (c). After stirring at room temperature overnight, the reaction mixture was treated with sodium hydrogen carbonate solution (20 ml) and the mixture stirred for 20 min. The layers were separated and sodium hydroxide solution (1M, 20 ml) added to the aqueous phase. The aqueous phase was then extracted three times with 10% methanol in dichloromethane (total volume 100 ml). The combined organic extracts were dried (MgSO₄) and evaporated. The residue was applied to a column of silica gel and eluted with 0-10% methanol in dichloromethane to afford the desired product as a white solid (323 mg, 69%).

MS (ES+), m/z 237 (MH⁺, 100%).

### (g) [(6S)-3-Fluoro-8-oxo-5,6-dihydro-8H-[1,4]oxazino[2,3,4-de]-1,5-naphthyridin-6-yl]methyl methanesulfonate

(6R)-3-fluoro-6-(hydroxymethyl)-5,6-dihydro-8H-[1,4]oxazino[2,3,4-de]-1,5-naphthyridin-8-one (236 mg, 1.0 mmol) was converted to the mesylate under the general conditions described for Example 7(d). The product was obtained as a white solid and used without further purification.

MS (ES+), m/z 315 (MH⁺, 100%).

### (h) 1,1-Dimethylethyl (1-{[(6R)-3-fluoro-8-oxo-5,6-dihydro-8H-[1,4]oxazino[2,3,4-de]-1,5-naphthyridin-6-yl]methyl}-4-piperidinyl)carbamate

[(6S)-3-Fluoro-8-oxo-5,6-dihydro-8H-[1,4]oxazino[2,3,4-de]-1,5-naphthyridin-6-yl]methyl methanesulfonate (320 mg, containing 1.0 mmol) was reacted with 1,1-dimethylethyl 4-piperidinylcarbamate (400 mg, 2.0 equiv) under the general conditions described for Example 7(e). After work-up, the residue was chromatographed on silica gel eluting with 0-10% 2M methanolic ammonia in dichloromethane. Product-containing fractions were combined and evaporated to an off-white solid (261 mg, 63%).

MS (ES+), m/z 419 (MH⁺, 100%).

### (e) (6R)-6-[(4-Amino-1-piperidinyl)methyl]-3-fluoro-5,6-dihydro-8H-[1,4]oxazino[2,3,4-de]-1,5-naphthyridin-8-one dihydrochloride

1,1-Dimethylethyl (1-{[(6R)-3-fluoro-8-oxo-5,6-dihydro-8H-[1,4]oxazino[2,3,4-de]-1,5-naphthyridin-6-yl]methyl}-4-piperidinyl)carbamate (1.51 g, 3.6 mmol) was dissolved in dichloromethane (30 ml) and treated dropwise with a solution of 4M HCl in 1,4-dioxane (10 ml, 40 mmol over a period of 10 min. After stirring at room temperature for 30 min, the mixture was evaporated under reduced pressure to yield a white free-flowing solid (1.88 g >100%). This was assumed to be the hydrated dihydrochloride salt.

MS (ES+), m/z 319 (MH⁺, 100%).

### (i) Title Compound

(6R)-6-[(4-Amino-1-piperidinyl)methyl]-3-fluoro-5,6-dihydro-8H-[1,4]oxazino[2,3,4-de]-1,5-naphthyridin-8-one dihydrochloride (2.98 g, containing 5.56 mmol of assumed di-hydrochloride) was dissolved in methanol (20 ml) containing acetic acid (0.2 ml) and treated with sodium acetate (1.36 g, 3.0 equiv) followed by 2,3-dihydro[1,4]dioxino[2,3-c]pyridine-7-carbaldehyde (918 mg, 5.56 mmol) (for a synthesis see WO2004058144, Example 2(c) or WO2003087098 Example 19(d)). After stirring at room temperature for 40 min, (polystyrylmethyl)trimethylammonium cyanoborohydride 4.0 mmoles/gm (5.56 g, 4.0 equiv) was added and the mixture stirred at RT overnight. The mixture was then filtered and the filtrate evaporated to dryness. Chromatography on silica gel eluting with a gradient of 0-15% 2M ammonia/methanol in DCM gave the title compound as a free base as a white foam (1.69 g, 65%).

¹H NMR (400 MHz, CDCl₃) δ 1.47-1.56 (2H, m), 1.88-1.95 (2H, m), 2.02-2.10 (1H, m), 2.13-2.20 (1H, m), 2.29 (1H, dt, J 2.4, 11.6 Hz), 2.42-2.62 (2H, m), 2.85 (1H, d, J 11.2 Hz), 3.15 (1H, d, J 11.6 Hz), 3.84 (2H, s), 4.13 (1H, dd, J 0.8, 11.2 Hz), 4.27-4.29 (2H, m) 4.33-4.35 (2H, m), 4.92-4.96 (1H, m), 5.07 (1H, d, J 10.4 Hz), 6.82 (1H, s), 6.83 (1H, d, J 10.0 Hz), 7.86 (1H, d, J 10.0 Hz), 8.10 (1H, s), 8.37 (1H, d, J 2.0 Hz).

### Example 9

### (6R)-6-({4-[(2,3-Dihydro[1,4]dioxino[2,3-c]pyridin-7-ylmethyl)amino]-1-piperidinyl}methyl)-3-fluoro-5,6-dihydro-8H-[1,4]oxazino[2,3,4-de]-1,5-naphthyridin-8-one hydrochloride

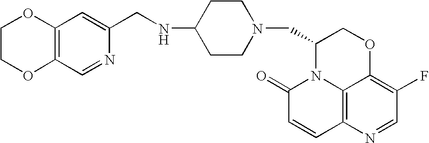

(6R)-6-({4-[(2,3-Dihydro[1,4]dioxino[2,3-c]pyridin-7-ylmethyl)amino]-1-piperidinyl}methyl)-3-fluoro-5,6-dihydro-8H-[1,4]oxazino[2,3,4-de]-1,5-naphthyridin-8-one free base (1.69 g) was dissolved in methanol (30 ml) and treated with 1M HCl in diethyl ether (3.6 ml, 1.0 equiv). The resultant mixture was evaporated to dryness, then evaporated twice from diethyl ether to give the hydrochloride salt as a light pink solid (1.47 g)

MS (ES+), m/z 468 (MH⁺, 40%).

Chiral HPLC (Chiralpak AD 4.6×250 mm, 10 um, eluting with 100% ethanol) showed >98% ee.

### Example 10

### (6R)-6-({4-[(2,3-Dihydro[1,4]dioxino[2,3-c]pyridin-7-ylmethyl)amino]-1-piperidinyl}methyl)-3-fluoro-5,6-dihydro-8H-[1,4]oxazino[2,3,4-de]-1,5-naphthyridin-8-one dihydrochloride

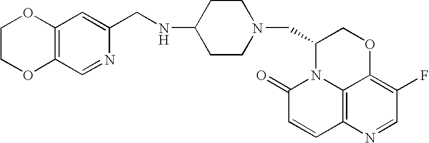

(6R)-6-({4-[(2,3-Dihydro[1,4]dioxino[2,3-c]pyridin-7-ylmethyl)amino]-1-piperidinyl}methyl)-3-fluoro-5,6-dihydro-8H-[1,4]oxazino[2,3,4-de]-1,5-naphthyridin-8-one free base (64 mg) was dissolved in methanol (2 ml) and treated with 1M HCl in diethyl ether (2 equivalents). The solution was evaporated to give the di-hydrochloride as an off-white solid.

MS (ES+), m/z 468 (MH⁺, 40%).

Chiral HPLC (ChiralpakAD 4.6×250 mm, 10 um, eluting with 100% ethanol) showed >99.8% ee.

### Example 11

### (6R)-6-({4-[(2,3-Dihydro[1,4]dioxino[2,3-c]pyridin-7-ylmethyl)amino]-1-piperidinyl}methyl)-3-fluoro-5,6-dihydro-8H-[1,4]oxazino[2,3,4-de]-1,5-naphthyridin-8-one benzoate

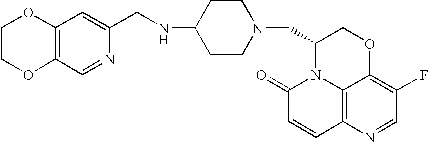

(6R)-6-({4-[(2,3-Dihydro[1,4]dioxino[2,3-c]pyridin-7-ylmethyl)amino]-1-piperidinyl}methyl)-3-fluoro-5,6-dihydro-8H-[1,4]oxazino[2,3,4-de]-1,5-naphthyridin-8-one free base (980 mg, 2.1 mmol) was dissolved in methanol (10 ml) and treated with a solution of benzoic acid (256 mg, 2.1 mmol) in methanol (2 ml). The solvent was evaporated under reduced pressure to give the benzoate salt as a white foam (1.2 g).

MS (ES+), m/z 468 (MH⁺, 100%).

### Example 12

### (3R)-3-({4-[(2,3-Dihydro[1,4]dioxino[2,3-c]pyridin-7-ylmethyl)amino]-1-piperidinyl}methyl)-10-fluoro-2,3-dihydro-5H-[1,4]oxazino[2,3,4-ij]quinolin-5-one dihydrochloride

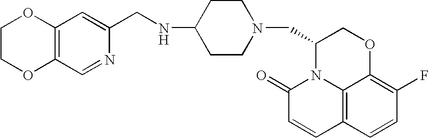

### (a) 7-Fluoro-2-methoxy-8-quinolinol

A solution of 8-bromo-7-fluoro-2-methoxyquinoline (for a preparation see Example 1(c)) (0.514 g, 2.0 mmol) in diethyl ether (30 ml) was cooled to −78° C. under argon. A solution of n-butyl lithium (2.5M in hexanes, 1.76 ml, 2.2 equiv) was added dropwise via syringe. After 5 mins the reaction mixture was treated dropwise with tri-isopropyl borate (1.02 ml, 2.2 equiv) and the mixture allowed to warm to room temperature over 30 mins. The mixture was then re-cooled to 0° C. and treated with aqueous hydrogen peroxide solution (35% w/w, 1.4 ml 5 equiv) The mixture was stirred at 0° C. for 15 min than at room temperature for 15 min. The reaction mixture was quenched with saturated aqueous ammonium chloride (20 ml), concentrated under reduced pressure, then extracted twice with dichloromethane. The combined organic extracts were washed with brine, dried (MgSO₄) and evaporate to an oil. This was chromatographed on silica eluting with 0-100% ethyl acetate in 40-60 petroleum ether. Product-containing fractions were combined and evaporated to a white solid (250 mg, 65%).

MS (ES+), m/z 194 (MH⁺, 100%).

### (b) (3R)-10-Fluoro-3-(hydroxymethyl)-2,3-dihydro-5H-[1,4]oxazino[2,3,4-ij]quinolin-5-one

7-Fluoro-2-methoxy-8-quinolinol (1.2 gms, 6.2 mmole) was dissolved in dry DMF (70 ml) and treated with NaH (248 mg, 6.2 mmole) and stirred at room temperature for 30 minutes. S-(+)-Glycidyl nosylate (1.93 g, 7.4 mmole) was added and the reaction stirred for 2 h. The reaction mixture was evaporated and the residue partioned between ethyl acetate and water. The organic phase was washed with saturated sodium bicarbonate, brine, and dried over magnesium sulphate, filtered then evaporated. Chromatography on silica gel eluting with a gradient of 0-100% ethyl acetate in hexane gave the title compound (0.56 g).

MS (ES+), m/z 236 (MH⁺, 100%).

### (c) [(3S)-10-Fluoro-5-oxo-2,3-dihydro-5H-[1,4]oxazino[2,3,4-ij]quinolin-3-yl]methyl methanesulfonate

(3R)-10-Fluoro-3-(hydroxymethyl)-2,3-dihydro-5H-[1,4]oxazino[2,3,4-ij]quinolin-5-one (1.36 g, 5.8 mmole) was dissolved in dry dichloromethane (100 ml), treated with triethylamine (0.97 ml) and cooled in an ice bath. Methanesulfonyl chloride (0.53 ml) was added and the ice bath removed and the reaction left to stir at room temperature for 20 min. The reaction mixture was washed with saturated sodium bicarbonate (100 ml), water (100 ml), and brine (100 ml). The organic phase was dried over magnesium sulphate, filtered and evaporated to dryness to give a pale yellow solid.

MS (ES+), m/z 314 (MH⁺, 100%).

### (d) 1,1-Dimethylethyl (1-{[(3R)-10-fluoro-5-oxo-2,3-dihydro-5H-[1,4]oxazino[2,3,4-ij]quinolin-3-yl]methyl}-4-piperidinyl)carbamate

[(3S)-10-Difluoro-5-oxo-2,3-dihydro-5H-[1,4]oxazino[2,3,4-ij]quinolin-3-yl]methyl methanesulfonate (1.8 g, 5.7 mmole) was dissolved in dry acetonitrile (100 ml), treated with pyridine (0.92 ml) followed by 1,1-dimethylethyl 4-piperidinylcarbamate (2.3 g, 11.4 mmole). The reaction mixture was heated at 70° C. under an argon atmosphere overnight. The reaction mixture was diluted with ethyl acetate (100 ml), the organic phase was washed with water and brine, dried over magnesium sulphate, filtered and evaporated. Chromatography on silica gel eluting with gradient 0-10% 2M methanolic ammonia in dichloromethane, gave the desired compound as a white solid.

MS (ES+), m/z 418 (MH⁺, 100%).

### (e) (3R)-3-[(4-Amino-1-piperidinyl)methyl]-10-fluoro-2,3-dihydro-5H-[1,4]oxazino[2,3,4-ij]quinolin-5-one

1,1-Dimethylethyl (1-{[(3R)-10-fluoro-5-oxo-2,3-dihydro-5H-[1,4]oxazino[2,3,4-ij]quinolin-3-yl]methyl}-4-piperidinyl)carbamate (2.1 g, 5.0 mmole) was dissolved in dry dichloromethane (40 ml). 4M HCl in 1,4-dioxane (3.75 ml. 15 mmole) was added and the reaction mixture stirred at room temperature for 10 min. A further 4 equivalents of 4M HCl in 1,4-dioxane (5 ml) were added and the reaction stirred for further 20 min. The excess acid was removed in vacuo and the solid dried over high vacuum. The free base was liberated by dissolving the HCl salt in minimum amount of dichloromethane. 2M methanolic ammonia in methanol (25 ml) was added. The solution was concentrated, then portioned between 10% methanol in dichloromethane and water, saturated with potassium carbonate and extracted with 10% methanol in dichloromethane. The combined organic phases were dried over magnesium sulphate, filtered and concentrated in vacuo to yield the title compound (1.3 g, 82%).

MS (ES+), m/z 318 (MH⁺, 100%).

### (f) Title Compound

(3R)-3-[(4-Amino-1-piperidinyl)methyl]-10-fluoro-2,3-dihydro-5H[1,4]oxazino[2,3,4-ij]quinolin-5-one (0.29 g, 0.92 mmole) and 2,3-dihydro[1,4]dioxino[2,3-c]pyridine-7-carbaldehyde ((0.152 g, 0.92 mmol) (for a synthesis see WO2004058144, Example 2(c) or WO2003087098 Example 19(d)) were dissolved in methanol (25 ml) and treated with acetic acid (0.25 ml). This was treated with. (Polystyrylmethyl) trimethylammonium cyanoborohydride 4.0 mmoles/gm (0.92 g, 3.7 mmol) was added and the mixture stirred at RT overnight. The mixture was then filtered and the filtrate evaporated to dryness. Chromatography on silica gel eluting with a gradient of 0-10% 2M ammonia/methanol in DCM gave the free base of the title compound as a pale yellow foam. (0.21, g).

¹H NMR (400 MHz, CDCl₃) δ 1.72-1.85 (3H, m), 1.87-1.93 (2H, m), 2.16-2.19 (1H, m), 2.25-2.31 (1H, m), 2.37-2.41 (1H, m), 2.48-2.55 (1H, m), 2.59-2.63 (1H, m), 2.84-2.87 (1H, m), 3.18-3.21 (1H, m), 3.78 (2H, s), 4.00-4.4.03 (1H, m), 4.28-4.34 (4H, m), 4.95-4.99 (2H, m), 6.61 (1H, d, J 8 Hz), 6.82 (1H, s), 6.99-7.09 (1H, m), 7.10-7.13 (1H, m), 7.64 (1H, d, J 8 Hz), 8.10 (1H, s).

MS (ES+) m/z 467 (MH+, 100%).

The free base was dissolved in methanol (5 ml), 1M HCl in diethyl ether (1.34 ml, 3 equiv) was added dropwise and the solution concentrated in vacuo to give the title compound as a pale yellow solid.

### Example 13

### Ethyl (2E)-3-[(3R)-3-({4-[(2,3-dihydro[1,4]dioxino[2,3-c]pyridin-7-ylmethyl)amino]-1-piperidinyl}methyl)-10-fluoro-5-oxo-2,3-dihydro-5H-[1,4]oxazino[2,3,4-ij]quinolin-8-yl]-2-propenoate

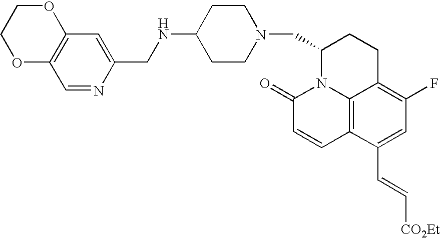

### (a) 5-Bromo-7-fluoro-2-(methyloxy)-8-quinolinol

To a solution of 7-fluoro-2-methoxy-8-quinolinol (0.23 g, 1.2 mmol) in THF (10 ml) was added NBS (0.23 g, 1.3 mmol) and the solution stirred for 1.5 h, then concentrated. Chromatography over silica (20 g SPE, eluting with 1:1 dichloromethane/hexane) provided the title compound as an off-white solid (0.28 g, 86%).

MS (ES+) m/z 272/274 [MH]⁺.

### (b) Ethyl (2E)-3-[7-fluoro-8-hydroxy-2-(methyloxy)-5-quinolinyl]-2-propenoate

To a solution of 5-bromo-7-fluoro-2-(methyloxy)-8-quinolinol (6.8 g, 25 mmol) in 1,4-dioxane (100 ml) was added ethyl acrylate (2.75 g, 27.5 mmol) followed by bis(tri-t-butylphosphine)palladium(0) (0.26 g), tris(dibenzylideneacetone)dipalladium (0.26 g) and dicyclohexylmethylamine (5.8 ml, 30 mmol). The mixture was degassed using argon then heated at 70° C. for 3 h, cooled and separated between ethyl acetate and brine. Chromatography over silica (100 g SPE, gradient elution using 1:1 dichloromethane/hexane to 100% dichloromethane) provided the title compound (4.8 g, 66%).

MS (ES+) m/z 292 [MH]⁺.

### (c) Ethyl (2E)-3-[(3R)-10-fluoro-3-(hydroxymethyl)-5-oxo-2,3-dihydro-5H-[1,4]oxazino[2,3,4-ij]quinolin-8-yl]-2-propenoate

To a solution of ethyl (2E)-3-[7-fluoro-8-hydroxy-2-(methyloxy)-5-quinolinyl]-2-propenoate (1.2 g, 4.1 mmol) in DMF (15 ml) was added sodium hydride (0.165 g, 4.1 mmol, 60% dispersal in paraffin). After 5 min (S)-glycidyl nosylate (1.07 g, 4.1 mmol) was added and the solution stirred for 28 h, then separated between ethyl acetate and water. The organics were isolated, washed with brine, dried and concentrated to an oil. Chromatography over silica (50 g SPE, gradient elution with dichloromethane to 1:1 dichloromethane/ethyl acetate) provided the title compound (0.65 g, 47%).

¹H NMR (400 MHz, CDCl₃) δ 1.36 (3H, t, J 7 Hz), 3.14 (1H, m), 3.77-3.84 (2H, m), 3.9 (1H, m), 4.13 (2H, q, J 7 Hz), 4.93 (1H, d, J 11.5 Hz), 5.14 (1H, m), 6.40 (1H, d, J 15.5 Hz), 6.75 (1H, d, J 10 Hz), 7.30 (1H, d, J 11 Hz), 8.10 (2H, m).

### (d) Ethyl (2E)-3-((3S)-10-fluoro-3-{[(methylsulfonyl)oxy]methyl}-5-oxo-2,3-dihydro-5H-[1,4]oxazino[2,3,4-ij]quinolin-8-yl)-2-propenoate

To a solution of ethyl (2E)-3-[(3R)-10-fluoro-3-(hydroxymethyl)-5-oxo-2,3-dihydro-5H-[1,4]oxazino[2,3,4-ij]quinolin-8-yl]-2-propenoate (0.63 g, 1.9 mmol) in dichloromethane (15 ml) was added triethylamine (0.27 ml, 1.9 mmol) followed by methanesulphonyl chloride (0.33 g, 1.9 mmol). The solution was stirred for 2 h then separated between dichloromethane and saturated sodium bicarbonate solution. The organics were isolated, dried and concentrated to provide the title compound as a cream solid (0.66 g, 85%).

MS (ES+) m/z 412 [MH]⁺.

### (e) Ethyl (2E)-3-((3R)-3-{[4-({[(1,1-dimethylethyl)oxy]carbonyl}amino)-1-piperidinyl]methyl}-10-fluoro-5-oxo-2,3-dihydro-5H-[1,4]oxazino[2,3,4-ij]quinolin-8-yl)-2-propenoate)

Ethyl (2E)-3-(3S)-10-fluoro-3-{[(methylsulfonyl)oxy]methyl}-5-oxo-2,3-dihydro-5H-[1,4]oxazino[2,3,4-ij]quinolin-8-yl)-2-propenoate (0.66 g, 1.6 mmol) was suspended in acetonitrile (20 ml) and 1,1-dimethylethyl-4-piperidinylcarbamate (0.32 g, 1.6 mmol) and potassium carbonate (0.66 g, 4.8 mmol) added. The mixture was heated at 70° C. for 18 h then cooled. The reaction mixture was diluted with ethyl acetate and washed with brine, dried and concentrated. Chromatography over silica (50 g SPE, gradient elution with dichloromethane/ethyl acetate 1:0 to 1:2) provided the title compound as a pale yellow oil (0.16 g, 19%).

MS (ES+) m/z 516 [MH]⁺.

### (f) Ethyl (2E)-3-{(3R)-3-[(4-amino-1-piperidinyl)methyl]-10-fluoro-5-oxo-2,3-dihydro-5H-[1,4]oxazino[2,3,4-ij]quinolin-8-yl}-2-propenoate dihydrochloride salt

Ethyl (2E)-3-(3R)-3-{[4-({[(1,1-dimethylethyl)oxy]carbonyl}amino)-1-piperidinyl]methyl}-10-fluoro-5-oxo-2,3-dihydro-5H-[1,4]oxazino[2,3,4-ij]quinolin-8-yl)-2-propenoate (0.16 g, 0.31 mmol) was dissolved in dichloromethane (3 ml) and a solution of hydrogen chloride in 1,4-dioxane (3 ml, 4M solution) added. The solution was stirred for 2 h then concentrated to provide the title compound as a white solid (0.15 g, 100%)

MS (ES+) m/z 384 [M-31]⁺.

### (g) Title Compound

Ethyl (2E)-3-{(3R)-3-[(4-amino-1-piperidinyl)methyl]-10-fluoro-5-oxo-2,3-dihydro-5H-[1,4]oxazino[2,3,4-ij]quinolin-8-yl}-2-propenoate dihydrochloride salt (0.15 g, 0.3 mmol) was suspended in dichloromethane (2 ml) and triethylamine (0.13 ml, 0.9 mmol) added. 2,3-Dihydro[1,4]dioxino[2,3-c]pyridine-7-carbaldehyde (0.051 g, 0.3 mmol) (for a synthesis see WO2004058144, Example 2(c) or WO2003087098 Example 19(d) was added to the resulting solution followed by sodium triacetoxyborohydride (0.32 g, 1.5 mmol) and stirred for 4 h, after which time further sodium triacetoxyborohydride (0.28 g, 1.3 mmol) was added and the solution stirred for 72 h. The solution was diluted with dichloromethane and washed with saturated sodium bicarbonate then brine. The organics were isolated, dried and concentrated to yield an oil. Chromatography over silica (5 g SPE, gradient elution with dichloromethane to dichloromethane/methanol 95:5) provided the title compound as an oil which on trituration under ether provided the product as a white solid (0.14 g, 81%).

MS (ES+) m/z 565 [MH]⁺.

### Example 14

### (2E)-3-[(3R)-3-({4-[(2,3-Dihydro[1,4]dioxino[2,3-c]pyridin-7-ylmethyl)amino]-1-piperidinyl}methyl)-10-fluoro-5-oxo-2,3-dihydro-5H-[1,4]oxazino[2,3,4-ij]quinolin-8-yl]-2-propenoic acid

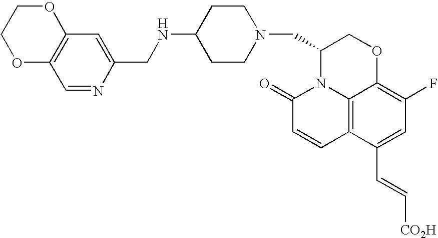

To a solution of ethyl (2E)-3-[(3R)-3-({4-[(2,3-dihydro[1,4]dioxino[2,3-c]pyridin-7-ylmethyl)amino]-1-piperidinyl}methyl)-10-fluoro-5-oxo-2,3-dihydro-5H-[1,4]oxazino[2,3,4-ij]quinolin-8-yl]-2-propenoate (0.12 g, 0.2 mmol) in 1,4-dioxane (2 ml) was added a solution of sodium hydroxide (0.034 g, 0.8 mmol) in water (2 ml). The solution was stirred for 4 h then concentrated and the residues suspended in water and acidified with hydrochloric acid (2M) to ˜pH6 whereupon a precipitate was formed and filtered off. The precipitate was washed with water then dried under vacuum to provide the title compound as a cream solid (0.076 g, 67%).

MS (ES+) m/z 537 [MH]⁺, m/z 535 [M-H].

### Example 15

### 6-({4-[(2,3-Dihydro[1,4]dioxino[2,3-c]pyridin-7-ylmethyl)amino]-1-piperidinyl}methyl)-3-fluoro-5,6-dihydro-4H,8H-pyrido[3,2,1-de]-1,5-naphthyridin-8-one dihydrochloride

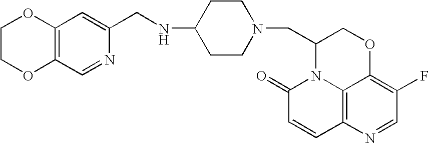

### (a) Butyl (2E)-3-[3-fluoro-6-(methoxy)-1,5-naphthyridin-4-yl]-2-propenoate

8-Bromo-7-fluoro-2-(methoxy)-1,5-naphthyridine (for a synthesis see WO2004058144 example 53(g)) (15.04 g 58.5 mmole) was dissolved in 1,4 dioxane (135 ml) and treated with tris(dibenzylidineacetone) dipalladium(0) (0.536 g 1 mole %) and bis(tri-t-butylphosphine)palladium (0) (0.598 g, 2 mole %). The reaction mixture was degassed by placing under vacuum and purging with argon several times. Butyl acrylate (8.39 ml 58.5 mmol) and dicyclohexylmethylamine (13.75 ml 64.35 mmol) were added and the reaction mixture was heated to 70° C. for 18 h. The reaction mixture was cooled, then treated with water (100 ml) and ethyl acetate (100 ml). The combined organic layers were dried over sodium sulphate, filtered, then evaporated to dryness. The residue was chromatographed on silica gel eluting with gradient of 5-10% ethyl acetate in hexane. This gave the title compound as a brown oil (13.58 g, 76%).

MS (ES+), m/z 305 (MH⁺, 100%).

¹H NMR (250 MHz, CDCl₃): δ 0.98-1.14 (3H, m), 1.39-1.52 (2H, m), 1.74-1.80 (2H, m), 4.14 (3H, s), 4.28-4.30 (2H, t, J 6.5 Hz), 7.11-7.15 (1H, d, J 10 Hz), 7.15-7.22 (1H, m), 8.17-8.23 (1H, m), 8.49-8.56 (1H, d, J 17 Hz), 8.71-8.72 (1H, m).

### (b) Butyl 3-[3-fluoro-6-(methyloxy)-1,5-naphthyridin-4-yl]propanoate

Butyl (2E)-3-[3-fluoro-6-(methyloxy)-1,5-naphthyridin-4-yl]-2-propenoate (42 g, 276 mmole) was split into two equal portions each of which was dissolved in ethanol (400 mL) and treated with 10% palladium on carbon paste (10 g) then hydrogenated at RT and one atmosphere of hydrogen for 24 h. The two portions were then re combined and filtered to remove catalyst. The filtrate was then evaporated to dryness to give the title compound.

### (c) 3-[3-Fluoro-6-(methyloxy)-1,5-naphthyridin-4-yl]-1-propanol

Butyl 3-[3-fluoro-6-(methyloxy)-1,5-naphthyridin-4-yl]propanoate (3.06 g, 10 mmol) was dissolved in dry THF (100 ml) and cooled to −78° C. under argon atmosphere. The solution was treated with a solution of lithium aluminium hydride (1.0 M solution in THF, 12 ml, 12 mmol) and stirred at −78° C. for 15 mins, warmed to 0° C. and stirred for a further 15 min. The mixture was cautiously treated with water (4 ml), 2M NaOH solution (7 ml) followed by water (8 ml) and stirred for a further 30 min Anhydrous sodium sulphate and diethyl ether (100 ml) were added and the mixture stirred for a further 30 min, filtered and evaporated. This was combined with the crude product from a reaction which had been prepared on a 1.0 mmole scale and purified by column chromatography eluting with a gradient of 0-100% ethyl acetate in 40-60 petroleum ether. This gave the title compound as an yellow solid (2.2 g, 85%).

MS (ES+), m/z 237 (MH⁺, 100%).

### (d) 3-[3-Fluoro-6-(methyloxy)-1,5-naphthyridin-4-yl]propanal

A solution of 3-[3-fluoro-6-(methyloxy)-1,5-naphthyridin-4-yl]-1-propanol (237 mg, 1.0 mmol) in dichloromethane (15 ml) was added to a solution of Dess-Martin periodinane 1.1 mmol, 0.47 g), and the reaction mixture stirred at room temperature for 20 min. Diethyl ether (50 ml) was added and the mixture poured into NaOH solution (1M, 15 ml) and stirred for 10 min. The layers were separated and the organic layer washed with 1M NaOH, water and brine, dried and evaporated to give the title compound as a white solid (215 mg, 91%).

MS (ES+), m/z 235 (MH⁺, 100%).

### (e) 7-Fluoro-2-(methyloxy)-8-[2-(2-oxiranyl)ethyl]-1,5-naphthyridine

3-[3-Fluoro-6-(methyloxy)-1,5-naphthyridin-4-yl]propanal (215 mg, 0.92 mmol) was converted to the title compound using the general conditions of Example 1(h). After chromatography on silica gel eluting with a gradient of 0-100% ethyl acetate in 40-60 petroleum ether, title compound was obtained as a colourless oil (75 mg, 33%).

MS (ES+), m/z 249 (MH⁺, 100%).

### (f) 3-Fluoro-6-(hydroxymethyl)-5,6-dihydro-4H,8H-pyrido[3,2,1-de]-1,5-naphthyridin-8-one

7-Fluoro-2-(methyloxy)-8-[2-(2-oxiranyl)ethyl]-1,5-naphthyridine (350 mg, 1.41 mmol) was dissolved in dichloromethane (25 ml) and treated with ytterbium(III) trifluoromethane sulphonate (300 mg, 0.35 equiv). The mixture was stirred at room temperature overnight then treated with a further 550 mg (0.65 mmol) of ytterbium(III) trifluoromethane sulphonate. Stirring was continued for a further 3 hours. The reaction mixture was worked up by the addition of water (10 ml) and saturated sodium bicarbonate solution (20 ml) and stirred for 40 min. The product was extracted by washing with dichloromethane (3×50 ml) followed by 10% methanol in dichloromethane (4×50 ml). The combined organic phases were dried (Mg SO₄) and evaporated. The residue was chromatographed on silica gel eluting with 0-10% methanol in dichloromethane to afford the title compound as a white solid (238 mg, 72%).

MS (ES+), m/z 235 (MH⁺, 100%).

### (g) 3-Fluoro-8-oxo-5,6-dihydro-4H,8H-pyrido[3,2,1-de]-1,5-naphthyridine-6-carbaldehyde

3-Fluoro-6-(hydroxymethyl)-5,6-dihydro-4H,8H-pyrido[3,2,1-de]-1,5-naphthyridin-8-one (238 mg, 1.0 mmol) was dissolved in dichloromethane (30 ml) and treated with Dess-Martin Periodinane (466 mg, 1.1 mmol). The mixture was stirred at room temperature for 20 min, then diluted with dichloromethane (50 ml) and washed with 10% aqueous sodium thiosulphate solution (2×50 ml), water and brine. The organic phase was dried (MgSO4) and chromatographed on silica eluting with 0-10% methanol in dichloromethane. The title compound was obtained as a colourless oil, contaminated with a small amount of Dess-Martin by-products (260 mg, >100%).

MS (ES+), m/z 265 ([M+MeOH]⁺, 100%), 232 (MH+, 30%).

### (h) 6-[(4-Amino-1-piperidinyl)methyl]-3-fluoro-5,6-dihydro-4H,8H-pyrido[3,2,1-de]-1,5-naphthyridin-8-one

3-Fluoro-8-oxo-5,6-dihydro-4H,8H-pyrido[3,2,1-de]-1,5-naphthyridine-6-carbaldehyde (260 mg, containing 1.0 mmol) was dissolved in methanol (40 ml) and reacted with 1,1-dimethylethyl 4-piperidinylcarbamate (0.2 g, 1.0 mmole) under the general conditions of Example 1(k). After work-up and chromatography, the BOC-protected amine was dissolved in dichloromethane (2 ml) and treated with TFA (2 ml). The reaction mixture was stirred at RT for 30 min, then evaporated. The residue was chromatographed on silica gel eluting with 0-20% 2M ammonia in methanol in DCM, to afford the title compound as a colourless oil (90 mg).

MS (ES+), m/z 317 (MH⁺ 100%).

### (i) Title Compound

6-[(4-Amino-1-piperidinyl)methyl]-3-fluoro-5,6-dihydro-4H,8H-pyrido[3,2,1-de]-1,5-naphthyridin-8-one (0.14 g, 0.44 mmole) and 2,3-dihydro[1,4]dioxino[2,3-c]pyridine-7-carbaldehyde (for a synthesis see WO2004058144, Example 2(c) or WO2003087098 Example 19(d)) (0.072 g, 0.44 mmole) were dissolved in methanol (20 mL) and acetic acid (0.02 ml) The reaction was stirred for 5 min and treated with (polystyrylmethyl)trimethylammonium cyanoborohydride 4.0 mmoles/gm (0.44 g, 1.76 mmoles) The reaction was stirred at RT overnight. The mixture was then filtered and the filtrate was evaporated to dryness.

Chromatography on silica gel eluting with a gradient of 0-10% 2M ammonia/methanol in DCM gave the free base of the title compound as a white foam (150 mg 73%).

¹H NMR (250 MHz, CDCl₃): δ 1.35-2.88 (14H, m), 3.13-3.21 (2H, m), 3.81 (2H, s), 4.25-4.38 (4H, m), 5.22-5.33 (1H, m), 6.80-6.85 (2H, m), 7.85 (1H, d, J 9.7 Hz), 8.10 (1H, s), 8.35 (1H, s).

MS (ES+), m/z 466 (MH⁺, 40%). 488 (MNa⁺, 10%).

The free base of the title compound (22 mg, 0.047 mmol) was dissolved in dichloromethane and treated with a solution of HCl in diethyl ether (0.2 ml, 0.2 mmol). A white precipitate formed which was collected by filtration and dried in vacuo, to give the title compound as a white solid (18 mg).

MS as for the free base.

### Example 16

### 6-({4-[(2,3-Dihydro[1,4]dioxino[2,3-c]pyridin-7-ylmethyl)amino]-1-piperidinyl}methyl)-3-fluoro-5,6-dihydro-4H,8H-pyrido[3,2,1-de]-1,5-naphthyridin-8-one Enantiomer 1 and

### Example 17

### 6-({4-[(2,3-Dihydro[1,4]dioxino[2,3-c]pyridin-7-ylmethyl)amino]-1-piperidinyl}methyl)-3-fluoro-5,6-dihydro-4H,8H-pyrido[3,2,1-de]-1,5-naphthyridin-8-one Enantiomer 2

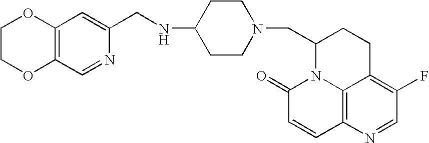

6-({4-[(2,3-dihydro[1,4]dioxino[2,3-c]pyridin-7-ylmethyl)amino]-1-piperidinyl}methyl)-3-fluoro-5,6-dihydro-4H,8H-pyrido[3,2,1-de]-1,5-naphthyridin-8-one (for a preparation see Example 15) (110 mg) was resolved into its separate enantiomers using preparative HPLC (multiple injections) on a Chiralpak AD-H column (21×250 mm) eluting with 50:50 acetonitrile:methanol containing 0.1% isopropylamine. This gave after evaporation of eluents Enantiomer 1 (39 mg) with a retention time of 7 mins and Enantiomer 2 (35 mg) with a retention time of 13 mins. Enantiomer 1 showed a negative peak on the HPLC polarimeter and 100% ee. Enantiomer 2 with a positive peak on the polarimeter also showed 100% ee.

### Example 18

### 6-({4-[(2,3-Dihydro[1,4]dioxino[2,3-c]pyridin-7-ylmethyl)amino]-1-piperidinyl}methyl)-3-fluoro-5,6-dihydro-4H,8H-pyrido[3,2,1-de]-1,5-naphthyridin-8-one Enantiomer 1 benzoate

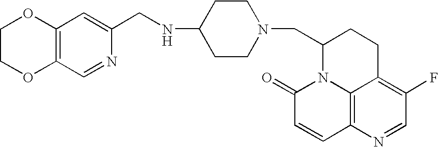

Racemic 6-({4-[(2,3-dihydro[1,4]dioxino[2,3-c]pyridin-7-ylmethyl)amino]-1-piperidinyl}methyl)-3-fluoro-5,6-dihydro-4H,8H-pyrido[3,2,1-de]-1,5-naphthyridin-8-one (for a preparation see Example 16) (253 mg) was resolved by chiral HPLC using the general conditions of Example 16. Enantiomer 1 (the first eluting isomer) was converted to the benzoate salt (134 mg).

### Example 19

### (6R)-6-({4-[(2,3-Dihydro[1,4]dioxino[2,3-c]pyridin-7-ylmethyl)amino]-1-piperidinyl}methyl)-8-oxo-5,6-dihydro-8H-[1,4]oxazino[2,3,4-de]-1,5-naphthyridine-3-carbonitrile diformate salt

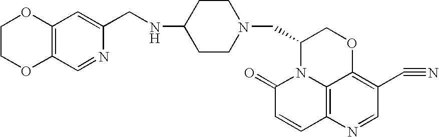

### (a) (6R)-6-(Hydroxymethyl)-8-oxo-5,6-dihydro-8H-[1,4]oxazino[2,3,4-de]-1,5-naphthyridine-3-carbonitrile

(6R)-3-Bromo-6-(hydroxymethyl)-5,6-dihydro-8H-[1,4]oxazino[2,3,4-de]-1,5-naphthyridin-8-one (for a synthesis see Example 7(c)) (150 mg, 0.5 mmol) was dissolved in dry DMF (5 ml), treated with copper (I) cyanide (56 mg, 0.6 mmol) and copper(I) iodide (42 mg, 0.21 mmol) and heated to 100° C. for 3 h. A further 6 equiv (280 mg) of copper (I) cyanide was added and the mixture heated to 120° C. for 18 h. The reaction mixture was cooled, evaporated and the residue partitioned between water (40 ml) and 15% methanol in dichloromethane (80 ml). The aqueous layer was further extracted with 15% methanol in dichloromethane (2×50 ml). The combined organic extracts were dried and evaporated to afford the title compound as a pale yellow solid (30 mg, 31%).

MS (ES+), m/z 244 (MH⁺ 100%).

### (b) [(6S)-3-Cyano-8-oxo-5,6-dihydro-8H-[1,4]oxazino[2,3,4-de]-1,5-naphthyridin-6-yl]methyl methanesulfonate

(6R)-6-(Hydroxymethyl)-8-oxo-5,6-dihydro-8H-[1,4]oxazino[2,3,4-de]-1,5-naphthyridine-3-carbonitrile (300 mg, 1.23 mmol) was suspended in dry dichloromethane (25 ml), treated with triethylamine (0.215 ml, 1.2 equiv) and cooled in an ice-water bath. Methane sulphonyl chloride (0.115 ml, 1.2 equiv) was added dropwise and the cooling bath removed. The reaction was stirred at room temperature for 1 h, diluted with dichloromethane, and washed with water, saturated sodium bicarbonate, and brine, dried (MgSO₄) and evaporated to afford the title compound as a solid (275 mg, 69%).

MS (ES+), m/z 322 (MH⁺ 100%).

### (c) 1,1-Dimethylethyl (1-{[(6R)-3-cyano-8-oxo-5,6-dihydro-8H-[1,4]oxazino[2,3,4-de]-1,5-naphthyridin-6-yl]methyl}-4-piperidinyl)carbamate

[(6S)-3-Cyano-8-oxo-5,6-dihydro-8H-[1,4]oxazino[2,3,4-de]-1,5-naphthyridin-6-yl]methyl methanesulfonate (271 mg, 0.84 mmol) was reacted with 1,1-dimethylethyl 4-piperidinylcarbamate (336 mg, 2.0 equiv) under the general conditions described in Example 7(e) to afford the title compound as a white solid after column chromatography (88 mg, 26%).

MS (ES+), m/z 426 (MH⁺ 100%).

### (d) Title Compound

1,1-Dimethylethyl (1-{[(6R)-3-cyano-8-oxo-5,6-dihydro-8H-[1,4]oxazino[2,3,4-de]-1,5-naphthyridin-6-yl]methyl}-4-piperidinyl)carbamate (86 mg, 0.2 mmol) was dissolved in dry dichloromethane (2 ml), treated with TFA (2 ml) and stirred at room temperature for 20 min. The solvent was evaporated, the residue taken up in dichloromethane and re-evaporated. The process was repeated three times to afford a white foam. This was dissolved in in methanol (2 mL), and acetic acid (0.02 ml) and treated with sodium acetate (anhydrous, 49 mg, 3.0 equiv) followed by 2,3-dihydro[1,4]dioxino[2,3-c]pyridine-7-carbaldehyde (for a synthesis see WO2004058144, Example 2(c) or WO2003087098 Example 19(d)) (0.046 g, 0.28 mmole). After stirring at room temperature for 40 min, (polystyrylmethyl)trimethylammonium cyanoborohydride 4.0 mmoles/gm (0.3 g, 1.2 mmol) was added and the mixture stirred at RT overnight. The mixture was then filtered and the filtrate evaporated to dryness. Chromatography on silica gel eluting with a gradient of 0-20% 2M ammonia/methanol in DCM gave the free base of the title compound as a colourless oil (40 mg). The presence of 20% of the corresponding 3-Bromo compound was noted as an impurity. The product was purified to >90% purity and converted to the di-formate salt by mass-directed preparative HPLC using 0.1% formic acid in acetonitrile/water as eluant to afford the title compound (15 mg).

¹H NMR (400 MHz, MeOD) δ 1.67-1.70 (2H, m), 2.06-2.30 (4H, m), 2.50-2.65 (2H, m), 3.01-3.20 (3H, m), 4.20 (2H, s), 4.31-4.33 (2H, m) 4.36-4.39 (2H, m), 4.44 (1H, dd, J 2.4, 11.6 Hz), 5.06-5.09 (2H, m), 7.00 (1H, s), 7.04 (1H, d, J 10.0 Hz), 8.01 (1H, d, J 10.0 Hz), 8.13 (1H, s), 8.25 (2H, br. s), 8.63 (1H, s).

MS (ES+), m/z 475 (MH⁺ 100%).

### Example 20

### (3R)-3-({(3R,4S)-4-[(2,3-Dihydro[1,4]dioxino[2,3-c]pyridin-7-ylmethyl)amino]-3-hydroxy-1-piperidinyl}methyl)-10-fluoro-2,3-dihydro-5H-[1,4]oxazino[2,3,4-ij]quinolin-5-one dihydrochloride

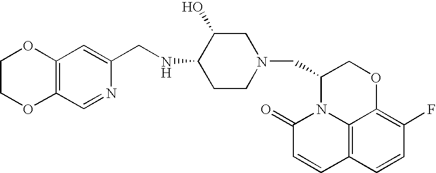

### (a) 1,1-Dimethylethyl ((3R,4S)-1-{[(3R)-10-fluoro-5-oxo-2,3-dihydro-5H-[1,4]oxazino[2,3,4-ij]quinolin-3-yl]methyl}-3-hydroxy-4-piperidinyl)carbamate

[(3S)-10-Fluoro-5-oxo-2,3,6,7-tetrahydro-5H-[1,4]oxazino[2,3,4-ij]quinolin-3-yl]methyl methanesulfonate (for a preparation see Example 12(c)) (1.0 gm, 3.2 mmol) was dissolved in dry acetonitrile (30 ml) and treated with dry pyridine (0.516 ml) and cis-(3-hydroxy-piperidin-4-yl)-carbamic acid tert-butyl ester Enantiomer 1 (for a synthesis see WO 2004/058144 Example 5(c)) (1.4 gm, 6.4 mmol). The reaction mixture was heated at 70° C. under an argon atmosphere overnight. The reaction mixture was diluted with ethyl acetate (100 ml); the organic phase was washed with water and brine, dried over magnesium sulphate, filtered and evaporated. Chromatography on silica gel eluting with gradient 0-10% 2M methanolic ammonia in dichloromethane, gave the desired compound as white foam (1.0 g).

MS (ES+), m/z 434 (MH⁺, 100%).

### (b) (3R)-3-{[(3R,4S)-4-Amino-3-hydroxy-1-piperidinyl]methyl}-10-fluoro-2,3-dihydro-5H-[1,4]oxazino[2,3,4-ij]quinolin-5-one

1,1-Dimethylethyl ((3R,4S)-1-{[(3R)-10-fluoro-5-oxo-2,3-dihydro-5H-[1,4]oxazino[2,3,4-ij]quinolin-3-yl]methyl}-3-hydroxy-4-piperidinyl)carbamate (1.0 g, 2.3 mmol) was dissolved in dry DCM (25 ml), treated with TFA (10 ml), and stirred at RT for 1 h. The excess solvent was removed in vacuo. Chromatography on silica gel eluting with a gradient of 0-20% 2M ammonia/methanol in DCM gave the title compound as white foam (0.7 g).

MS (ES+), m/z 334 (MH⁺, 100%).

### (c) Title Compound

(3R)-3-{[(3R,4S)-4-Amino-3-hydroxy-1-piperidinyl]methyl}-10-fluoro-2,3-dihydro-5H-[1,4]oxazino[2,3,4-ij]quinolin-5-one (0.05 g, 0.15 mmol) and 2,3-dihydro[1,4]dioxino[2,3-c]pyridine-7-carbaldehyde (for a synthesis see WO2004058144, Example 2(c) or WO2003087098 Example 19(d)) (0.025 g, 0.15 mmol) was dissolved in methanol (10 ml) and treated with acetic acid (0.1 ml). This was treated with. (polystyrylmethyl) trimethylammonium cyanoborohydride 4.0 mmoles/gm (0.2 g, 0.6 mmol) was added and the mixture stirred at RT overnight. The mixture was then filtered and the filtrate evaporated to dryness. Chromatography on silica gel eluting with a gradient of 0-10% 2M ammonia/methanol in DCM gave the title compound as a free base as a pale yellow oil. (Yield 0.05 g).

¹H NMR (400 MHz, CDCl₃) ∂ 2.04-2.13 (3H, m), 2.30-2.35 (2H, m), 2.45-2.49 (1H, m), 2.65-2.71 (1H, m), 2.83-2.85 (1H, m), 3.22-3.25 (1H, m), 3.78 (3H, s), 4.03-4.05 (2H, m), 4.25-4.35 (5H, m), 4.91-5.02 (2H, m), 6.62 (1H, d, J 12 Hz), 6.83 (1H, s), 6.95-7.00 (1H, m), 7.02-7.11 (1H, m), 7.64 (1H, d, J 12 Hz), 8.10 (1H, s).

MS (ES+), m/z 483 (MH⁺, 10%).

The free base of the title compound was dissolved in methanol (2 ml) 1M HCl in diethyl ether 3 equivalents. (0.35 ml) was added dropwise and the solution concentrated in vacuo to give the title compound as a solid. (Yield 0.06 g).

### Example 21

### (3R)-3-[((3S,4S)-3-{[(2,3-Dihydro[1,4]dioxino[2,3-c]pyridin-7-ylmethyl)amino]methyl}-4-hydroxy-1-pyrrolidinyl)methyl]-10-fluoro-2,3-dihydro-5H-[1,4]oxazino[2,3,4-ij]quinolin-5-one dihydrochloride

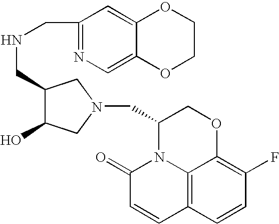

### (a) Phenylmethyl {[(3R,4S)-4-hydroxy-3-pyrrolidinyl]methyl}carbamate

To a stirred solution of 1,1-dimethylethyl (3S,4S)-3-hydroxy-4-[({[(phenylmethyl)oxy]carbonyl}amino)methyl]-1-pyrrolidinecarboxylate (for a synthesis see WO2006002047 preparation 24(c), (±)-1,1-dimethylethyl cis-3-hydroxy-4-[({[(phenylmethyl)oxy]carbonyl}amino)methyl]-1-pyrrolidinecarboxylate E1 isomer) (1 g, 2.85 mmol) in DCM (50 ml) was added trifluoracetic acid (50 ml) and stirred for 2 h. The reaction mixture was concentrated and placed under high vacuum for 3 h. To the TFA salt dissolved in 100 ml of 10:1 CHCl₃:MeOH was added MP-carbonate resin (4 g; 11.4 mmol) and stirred overnight. The reaction mixture was filtered and concentrated to provide the title compound as a pale yellow oil (840 mg, 100%)

MS (ES+) m/z 251.3 (MH+).

### (b) Phenylmethyl [((3S,4S)-1-{[(3R)-10-fluoro-5-oxo-2,3-dihydro-5H-[1,4]oxazino[2,3,4-ij]quinolin-3-yl]methyl}-4-hydroxy-3-pyrrolidinyl)methyl]carbamate

[(3S)-10-Fluoro-5-oxo-2,3,6,7-tetrahydro-5H-[1,4]oxazino[2,3,4-ij]quinolin-3-yl]methyl methanesulfonate (for a preparation see Example 12(c)) (0.5 gm, 1.6 mmol) was dissolved in dry acetonitrile (20 ml) and treated with dry pyridine (0.181 ml) and phenylmethyl {[(3R,4S)-4-hydroxy-3-pyrrolidinyl]methyl}carbamate (0.48 g, 1.92 mmol). The reaction mixture was heated at 60° C. under argon atmosphere overnight. The reaction mixture was diluted with ethyl acetate (100 ml); the organic phase was washed with water and brine, dried over magnesium sulphate, filtered and evaporated. Chromatography on silica gel eluting with gradient 0-10% 2M methanolic ammonia in dichloromethane, gave the desired compound as white foam (0.37 g).

MS (ES+), m/z 468 (MH⁺, 100%).

### (c) (3R)-3-{[(3S,4S)-3-(Aminomethyl)-4-hydroxy-1-pyrrolidinyl]methyl}-10-fluoro-2,3-dihydro-5H-[1,4]oxazino[2,3,4-ij]quinolin-5-one

Phenylmethyl [((3S,4S)-1-{[(3R)-10-fluoro-5-oxo-2,3-dihydro-5H-[1,4]oxazino[2,3,4-ij]quinolin-3-yl]methyl}-4-hydroxy-3-pyrrolidinyl)methyl]carbamate (0.36 g, 0.77 mmol) was dissolved in ethanol (30 ml) and treated with 10% palladium on carbon paste (0.3 g) then hydrogenated at RT and one atmosphere of hydrogen for 2 h. The reaction mixture was filtered to remove catalyst. The filtrate was then evaporated to dryness to give the title compound as colourless oil. (Yield 0.185 g.)

MS (ES+), m/z 334 (MH⁺, 100%).

### (d) Title Compound

A mixture of (3R)-3-{[(3S,4S)-3-(aminomethyl)-4-hydroxy-1-pyrrolidinyl]methyl}-10-fluoro-2,3-dihydro-5H-[1,4]oxazino[2,3,4-ij]quinolin-5-one (35 mg, 0.1 mmol) and 2,3-dihydro[1,4]dioxino[2,3-c]pyridine-7-carboxaldehyde (0.94 g, 5.7 mmole) (for a synthesis see WO2004058144, Example 2(c) or WO2003087098 Example 19(d)) (0.017 g, 0.1 mmole) in methanol (2 mL) and acetic acid (20 microliters) was treated with (polystyrylmethyl)trimethylammonium cyanoborohydride 4.0 mmoles/gm (0.1 g, 0.4 mmoles) and stirred at RT for 18 h. The mixture was then filtered and evaporated to dryness. Chromatography on silica gel eluting with a gradient of 0-20% 2M ammonia/methanol in DCM gave the free base of the title compound as a colourless oil (0.018 g).

¹H NMR (400 MHz, CDCl₃): δ 2.35-2.41 (1H, m), 2.43-2.47 (1H, m), 2.54-2.62 (1H, m), 2.78-2.93 (6H, m), 3.78 (2H, s), 3.97-4.03 (1H, m), 4.28-4.29 (2H, m), 4.32-4.34 (2H, m), 4.45-4.48 (1H, m), 4.96 (2H, d, J 11.2 Hz), 4.98-5.02 (1H, m), 6.59 (1H, d, J 9.6 Hz), 6.76 (1H, s), 6.95-7.00 (1H, m), 7.12-7.15 (1H, m), 7.64 (1H, d, J 9.6 Hz), 8.13 (1H, s).

MS (ES+), m/z 483 (MH⁺, 40%).

The free base of the title compound (18 mg, 0.037 mmol) was dissolved in methanol (2 ml) and treated with a solution of HCl in diethyl ether (0.13 ml, 3 equiv). The solvent was evaporated to afford the title product as a pale yellow solid.

MS as for the free base.

### Example 22

### (6R)-6-({(3R,4S)-4-[(2,3-Dihydro[1,4]dioxino[2,3-c]pyridin-7-ylmethyl)amino]-3-hydroxy-1-piperidinyl}methyl)-3-fluoro-5,6-dihydro-8H-[1,4]oxazino[2,3,4-de]-1,5-naphthyridin-8-one dihydrochloride salt

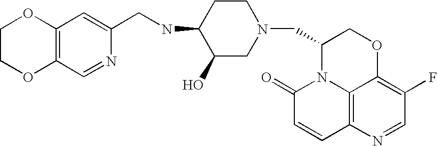

### (a) 1,1-Dimethylethyl ((3R,4S)-1-{[(6R)-3-fluoro-8-oxo-5,6-dihydro-8H-[1,4]oxazino[2,3,4-de]-1,5-naphthyridin-6-yl]methyl}-3-hydroxy-4-piperidinyl)carbamate

[(6S)-3-Fluoro-8-oxo-5,6-dihydro-8H-[1,4]oxazino[2,3,4-de]-1,5-naphthyridin-6-yl]methyl methanesulfonate (for a preparation see Example 8(g)) (0.8 g, 2.5 mmol) was converted to the title compound on treatment with cis-(3-hydroxy-piperidin-4-yl)-carbamic acid tert-butyl ester Enantiomer 1 (for a synthesis see WO 2004/058144 example 5(c)) (0.650 g, 3.25 mmol) under the standard conditions described for Example 7(e). After work-up and chromatography the desired product was obtained as a white foam (0.444 g).

MS (ES+), m/z 435 (MH⁺, 80%).

### (b) (6R)-6-{[(3R,4S)-4-Amino-3-hydroxy-1-piperidinyl]methyl}-3-fluoro-5,6-dihydro-8H-[1,4]oxazino[2,3,4-de]-1,5-naphthyridin-8-one trifluoroacetate salt

1,1-Dimethylethyl ((3R,4S)-1-{[(6R)-3-fluoro-8-oxo-5,6-dihydro-8H-[1,4]oxazino[2,3,4-de]-1,5-naphthyridin-6-yl]methyl}-3-hydroxy-4-piperidinyl)carbamate (0.44 g, 1 mmol) was treated with trifluoroacetic acid (5 mL) in DCM (20 mL) for 18 h at RT then evaporated to dryness. The residue was dissolved in methanol (50 mL) and Amberlyst A21 ion exchange resin (approx 15 g) was added and the mixture stirred for 1 h to remove excess TFA. The mixture was then filtered and the filtrate evaporated to dryness to give the title compound, believed to be the mono TFA salt as a white foam (0.44 g).

MS (ES+), m/z 335 (MH⁺, 100%).

### (c) Title Compound

(6R)-6-{[(3R,4S)-4-Amino-3-hydroxy-1-piperidinyl]methyl}-3-fluoro-5,6-dihydro-8H-[1,4]oxazino[2,3,4-de]-1,5-naphthyridin-8-one trifluoroacetate salt (0.22 g, 0.49 mmol) was treated with 2,3-dihydro[1,4]dioxino[2,3-c]pyridine-7-carbaldehyde (0.081 g, 0.49 mmol) (for a synthesis see WO2004058144, Example 2(c) or WO2003087098 Example 19(d)) under the general conditions of Example 8(f). The free base of the title compound was obtained as a colourless oil (0.162 g).

1H NMR (250 MHz, CDCl₃). δ 1.59-1.83 (2H, m), 2.27-2.41 (2H, m), 2.43-2.73 (4H, m), 2.75-2.85 (1H, m), 3.17-3.27 (1H, m), 3.81 (2H, s), 4.09-4.19 (1H, m), 4.25-4.38 (4H, m), 4.91-5.01 (1H, m), 5.05 (1H, d, J 11 Hz), 6.79 (1H, s), 6.82 (1H, d, J 10 Hz), 7.85 (1H, d, J 10 Hz), 8.11 (1H, s), 8.38 (1H, d, J 2 Hz).

MS (ES+) m/z 484 (MH+, 60%)

The free base of the title compound was dissolved in methanol (5 mL) and treated with excess 4M hydrogen chloride in 1,4-dioxane then evaporated to dryness to give the title compound as a white solid.

### Example 23

### (6R)-3-Fluoro-6-{[(3S,4S)-3-hydroxy-4-({[(3-oxo-3,4-dihydro-2H-pyrido[3,2-b][1,4]oxazin-6-yl)methyl]amino}methyl)-1-pyrrolidinyl]methyl}-5,6-dihydro-8H-[1,4]oxazino[2,3,4-de]-1,5-naphthyridin-8-one dihydrochloride salt

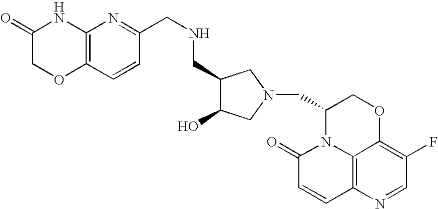

### (a) Phenylmethyl [((3S,4S)-1-{[(6R)-3-fluoro-8-oxo-5,6-dihydro-8H-[1,4]oxazino[2,3,4-de]-1,5-naphthyridin-6-yl]methyl}-4-hydroxy-3-pyrrolidinyl)methyl]carbamate

[(6S)-3-Fluoro-8-oxo-5,6-dihydro-8H-[1,4]oxazino[2,3,4-de]-1,5-naphthyridin-6-yl]methyl methanesulfonate (0.214 g, 0.68 mmol) was converted to the title compound on treatment with phenylmethyl {[(3R,4S)-4-hydroxy-3-pyrrolidinyl]methyl}carbamate (for a preparation see Example 21(a)) (0.256 g, 1 mmol) under the standard conditions described for Example 7(e). After work-up and chromatography the desired product was obtained as a colourless oil (0.152 g).

MS (ES+), m/z 469 (MH⁺, 100%).

### (b) (6R)-6-{[(3S,4S)-3-(Aminomethyl)-4-hydroxy-1-pyrrolidinyl]methyl}-3-fluoro-5,6-dihydro-8H-[1,4]oxazino[2,3,4-de]-1,5-naphthyridin-8-one

Phenylmethyl [((3S,4S)-1-{[(6R)-3-fluoro-8-oxo-5,6-dihydro-8H-[1,4]oxazino[2,3,4-de]-1,5-naphthyridin-6-yl]methyl}-4-hydroxy-3-pyrrolidinyl)methyl]carbamate (0.152 g, 0.32 mmol) was dissolved in ethanol (30 mL) and hydrogenated at S.T.P over a 10% palladium on charcoal paste catalyst for 2 h. The catalyst was filtered off and the filtrate evaporated to dryness to give the title compound (0.116 g).

MS (ES+), m/z 335 (MH⁺, 100%).

### (c) Title Compound

(6R)-6-{[(3S,4S)-3-(Aminomethyl)-4-hydroxy-1-pyrrolidinyl]methyl}-3-fluoro-5,6-dihydro-8H-[1,4]oxazino[2,3,4-de]-1,5-naphthyridin-8-one (0.055 g, 0.16 mmol) was treated with 3-oxo-3,4-dihydro-2H-pyrido[3,2-b][1,4]oxazine-6-carbaldehyde (0.029 g, 0.16 mmol) (for a synthesis see WO2003087098 Example 31(e)) under the general conditions of Example 8(j). The free base of the title compound was obtained as a colourless oil (0.035 g).

1H NMR (250 MHz, CDCl₃). δ 2.33-2.47 (1H, m), 2.52-2.58 (1H, m), 2.69-2.92 (8H, m), 3.69-3.82 (2H, m), 4.09-4.19 (1H, m), 4.39-4.47 (1H, m), 4.64 (2H, s), 4.95-5.02 (1H, m), 5.04 (1H, d, J 11 Hz), 6.85 (1H, d, J 8 Hz), 6.89 (1H, d, J 10 Hz), 7.22 (1H, d, J 8 Hz), 7.87 (1H, d, J 10 Hz), 8.39 (1H, d, J 2 Hz).

MS (ES+) m/z 497 (MH+, 100%).

The free base of the title compound was dissolved in methanol (5 mL) and treated with excess 4M hydrogen chloride in 1,4-dioxane then evaporated to dryness to give the title compound as a white solid.

### Example 24

### (6R)-6-({(3S,4R)-4-[(2,3-Dihydro[1,4]dioxino[2,3-c]pyridin-7-ylmethyl)amino]-3-hydroxy-1-piperidinyl}methyl)-3-fluoro-5,6-dihydro-8H-[1,4]oxazino[2,3,4-de]-1,5-naphthyridin-8-one

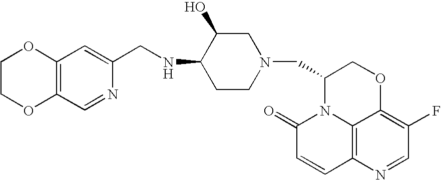

### (a) 1,1-Dimethylethyl ((3S,4R)-1-{[(6R)-3-fluoro-8-oxo-5,6-dihydro-8H-[1,4]oxazino[2,3,4-de]-1,5-naphthyridin-6-yl]methyl}-3-hydroxy-4-piperidinyl)carbamate

[(6S)-3-Fluoro-8-oxo-5,6-dihydro-8H-[1,4]oxazino[2,3,4-de]-1,5-naphthyridin-6-yl]methyl methanesulfonate (for a preparation see Example 8(g) (0.5 g, 1.58 mmol) was converted to the title compound on treatment with cis-(3-hydroxy-piperidin-4-yl)-carbamic acid tert-butyl ester Enantiomer 2 (for a synthesis see WO 2004/058144 Example 5(c)) (0.650 g, 3.25 mmol) under the general conditions described for Example 7(e). After work-up and chromatography the desired product was obtained as a white foam (523 mg, 76%).

MS (ES+), m/z 435 (MH⁺, 100%).

### (b) (6R)-6-{[(3S,4R)-4-Amino-3-hydroxy-1-piperidinyl]methyl}-3-fluoro-5,6-dihydro-8H-[1,4]oxazino[2,3,4-de]-1,5-naphthyridin-8-one dihydrochloride

1,1-Dimethylethyl ((3S,4R)-1-{[(6R)-3-fluoro-8-oxo-5,6-dihydro-8H-[1,4]oxazino[2,3,4-de]-1,5-naphthyridin-6-yl]methyl}-3-hydroxy-4-piperidinyl)carbamate (0.512 g, 1.17 mmol) was dissolved in dichloromethane (12 ml) and treated with a solution of 4M HCl in 1,4-dioxane (3.5 ml, 14 mmol). After stirring at room temperature for 30 min the solvent was evaporated to afford the title compound as a white solid (550 mg, >100%).

MS (ES+), m/z 335 (MH⁺, 100%).

### (c) Title Compound

(6R)-6-{[(3S,4R)-4-Amino-3-hydroxy-1-piperidinyl]methyl}-3-fluoro-5,6-dihydro-8H-[1,4]oxazino[2,3,4-de]-1,5-naphthyridin-8-one dihydrochloride (0.055 g, containing 0.117 mmol) was converted to the free base of the title compound by reaction with 2,3-dihydro[1,4]dioxino[2,3-c]pyridine-7-carbaldehyde (for a synthesis see WO2004058144, Example 2(c) or WO2003087098 Example 19(d)) (20 mg) under the general conditions of Example 8(j). The free base of the title compound was obtained as a colourless oil (20 mg).

¹H NMR (400 MHz, CDCl₃) δ 1.62-1.72 (2H, m), 1.88-1.95 (2H, m), 2.13-2.21 (1H, m), 2.40-2.60 (3H, m), 3.00-3.07 (2H, m), 3.82 (2H, s), 3.86 (1H, br.s) 4.16 (1H, dd, J 0.8, 12.0 Hz), 4.27-4.29 (2H, m) 4.33-4.35 (2H, m), 4.92-5.02 (2H, m), 6.85 (1H, s), 6.86 (1H, d, J 10.0 Hz), 7.86 (1H, d, J 10.0 Hz), 8.10 (1H, s), 8.37 (1H, d, J 2.0 Hz).

MS (ES+), m/z 484 (MH⁺, 60%).

The free base of the title compound (20 mg) was dissolved in methanol (2 ml) and treated with 1M HCl in methanol (0.2 ml, 0.2 mmol). The solid was evaporated to give the title compound as a pale yellow solid.

### Example 25

### (6R)-6-({(3S,4R)-4-[(3,4-Dihydro-2H-pyrano[2,3-c]pyridin-6-ylmethyl)amino]-3-hydroxy-1-piperidinyl}methyl)-3-fluoro-5,6-dihydro-8H-[1,4]oxazino[2,3,4-de]-1,5-naphthyridin-8-one

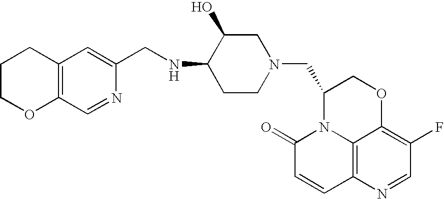

(6R)-6-{[(3S,4R)-4-Amino-3-hydroxy-1-piperidinyl]methyl}-3-fluoro-5,6-dihydro-8H-[1,4]oxazino[2,3,4-de]-1,5-naphthyridin-8-one dihydrochloride (for a preparation see Example 24(b)) (0.055 g, containing 0.117 mmol) was converted to the free base of the title compound by reaction with 3,4-dihydro-2H-pyrano[2,3-c]pyridine-6-carbaldehyde (for a synthesis see WO2004058144 Example 126(e)) (20 mg) under the general conditions of Example 8(j). The free base of the title compound was obtained as a colourless oil (15 mg).

¹H NMR (400 MHz, CDCl₃) δ 1.68-1.72 (2H, m), 1.78-1.93 (2H, m), 2.00-2.06 (2H, m), 2.18-2.26 (1H, m), 2.38-2.42 (1H, m) 2.48-2.60 (3H, m), 3.60 (2H, t, J 11.2 Hz), 2.94-3.03 (2H, m), 3.84-3.88 (2H, m) 4.16-4.22 (3H, m), 4.88-5.02 (2H, m), 6.82 (1H, d, J 10.0 Hz), 6.99 (1H, s), 7.88 (1H, d, J 10.0 Hz), 8.07 (1H, s), 8.38 (1H, d, J 2.0 Hz).

MS (ES+), m/z 482 (MH⁺, 100%).

The free base of the title compound (15 mg) was dissolved in methanol (2 ml) and treated with 1M HCl in methanol (0.2 ml, 0.2 mmol). The solid was evaporated to give the title compound as a pale yellow solid.

### Example 26

### (6R)-6-[(3-{[(2,3-Dihydro[1,4]dioxino[2,3-c]pyridin-7-ylmethyl)amino]methyl}-4-hydroxy-1-piperidinyl)methyl]-3-fluoro-5,6-dihydro-8H-[1,4]oxazino[2,3,4-de]-1,5-naphthyridin-8-one dihydrochloride salt

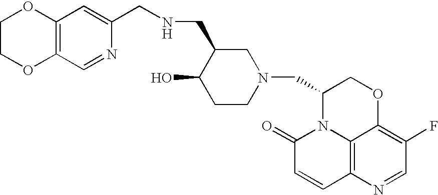

### (a) Racemic ethyl 4-hydroxy-1-(phenylmethyl)-3-piperidinecarboxylate (mixture of cis and trans)

To a solution of ethyl 4-oxo-1-(phenylmethyl)-3-piperidinecarboxylate hydrochloride (50 g, 170 mmol) in methanol (1 l) was added triethylamine (28.3 ml, 204 mmol) and the mixture stirred at room temperature for 10 min under argon. Sodium borohydride (21.32 g, 560 mmol) was then added portionwise and the reaction stirred at room temperature for 3 h. 5N HCl solution (175 ml) was added (final pH=2-3) and the mixture reduced to approx. 200 ml. The residue was neutralized with a saturated solution of sodium bicarbonate (150 ml) and the aqueous phase was extracted with dichloromethane and then a 9:1 dichloromethane:methanol mixture. The organic layer was dried and the solvent was removed under reduced pressure. This provided the desired compound (37 g, 84%) as a mixture of cis and trans isomers in an approximately 1:1 ratio.

MS (ES+) m/z 264 (MH⁺, 100%).

### (b) Ethyl (cis)-4-hydroxy-1-(phenylmethyl)-3-piperidinecarboxylate

To a solution of racemic ethyl 4-hydroxy-1-(phenylmethyl)-3-piperidinecarboxylate (37 g, 140 mmol) in N,N-dimethylformamide (250 ml) was added tert-butyldimethylchlorosilane (10.6 g, 70 mmol) and imidazole (5.3 g, 77 mmol) under argon. The reaction was stirred at room temperature for 3 h; water was added and the aqueous phase was extracted with dichloromethane. The organic layer was dried and the solvent was removed under reduced pressure to afford 40 g of crude. The crude was divided into 3 batches (5 g, 17 g, 17 g) and each batch was subjected to column chromatography on silica gel using a hexane and ethyl acetate gradient (0-20% ethyl acetate in hexane). This provided the desired compound (16.67 g, 45%) and ethyl (trans)-4-{[(1,1-dimethylethyl)(dimethyl)silyl]oxy}-1-(phenylmethyl)-3-piperidinecarboxylate (15.8 g, 30%).

MS (ES+) m/z 264 (MH⁺, 100%).

### (c) (cis)-4-Hydroxy-1-(phenylmethyl)-3-piperidinecarboxylic acid sodium salt

To a solution of ethyl (cis)-4-hydroxy-1-(phenylmethyl)-3-piperidinecarboxylate (16.67 g, 63.4 mmol) in THF/water (500 ml/50 ml) was added 2N sodium hydroxide solution (72 ml). The reaction mixture was stirred at room temperature for 5 h and then the pH was adjusted to 7 with 2N hydrochloric acid solution. The mixture was reduced to approx 50 ml and the solid formed was filtered off, washed with water and dried in vacuo to afford the desired compound as its sodium salt (17 g, >100%).

MS (ES+) m/z 236 (MH⁺, 100%).

### (d) (cis)-4-Hydroxy-1-(phenylmethyl)-3-piperidinecarboxamide

To a solution of (cis)-4-hydroxy-1-(phenylmethyl)-3-piperidinecarboxylic acid sodium salt (17 g) and 1-hydroxy-7-azabenzotriazole (5 g, 37 mmol) in N,N-dimethylformamide was added N-[3-(dimethylamino)propyl]-N′-ethylcarbodiimide hydrochloride (14.1 g, 73.4 mmol), followed by ammonium bicarbonate (21 g, 26.6 mmol). The reaction mixture was stirred at room temperature for 18 h. The solvent was then removed under reduced pressure and the residue was treated with aqueous sodium bicarbonate solution. The aqueous phase was extracted with 9:1 dichloromethane:methanol mixture. The organic layer was dried (MgSO4) and the solvent was removed under reduced pressure. The residue was subjected to column chromatography on silica gel using a methanol and dichloromethane gradient (0-20% methanol/dichloromethane). This provided the desired compound (9.5 g, 62%).

MS (ES+) m/z 235 (MH⁺, 80%), 257 (100%).

### (e) (cis)-3-(Aminomethyl)-1-(phenylmethyl)-4-piperidinol

A solution of (cis)-4-hydroxy-1-(phenylmethyl)-3-piperidinecarboxamide (0.7 g, 3 mmol) in THF (6 ml) was treated with a borane-methyl sulphide complex (2M solution in THF, 3.3 ml). The reaction mixture was heated at 80° C. for 1300 s under microwave irradiation conditions. This was repeated twelve times and then the reaction mixtures were combined and the solvent was removed under reduced pressure and the residue was subjected to column chromatography on silica gel using a dichloromethane, methanol and aqueous ammonia gradient (20% methanol/dichloromethane-20% 2M ammonia in methanol/dichloromethane) to provide 4.6 g of the desired pure compound and 1.1 g of less pure material.

MS (ES+) m/z 219 (MH⁺, 100%).

### (f) 1,1-Dimethylethyl {[(cis)-4-hydroxy-1-(phenylmethyl)-3-piperidinyl]methyl}carbamate

(cis)-3-(Aminomethyl)-1-(phenylmethyl)-4-piperidinol (4.6 g, 21.1 mmol) was dissolved in chloroform (125 ml) and stirred over sodium bicarbonate (4.5 g, 53.6 mmol). A solution of di-tent-butyl dicarbonate (4.64 g, 21.1 mmol) in chloroform (60 ml) was then added over 0.5 h and the reaction mixture stirred at room temperature. Water was added and the two phases separated. The aqueous phase was re-extracted with 9:1 dichloromethane:methanol mixture. The combined organic fractions were dried and the solvent was removed under reduced pressure. The residue was subjected to column chromatography on silica gel using a methanol and dichloromethane gradient (0-10% methanol in dichloromethane). This provided the desired compound (5 g, 75%).

MS (ES+) m/z 321 (MH⁺, 40%), 265 (100%).

### (g) 1,1-Dimethylethyl {[(cis)-4-hydroxy-1-(phenylmethyl)-3-piperidinyl]methyl}carbamate, Enantiomers 1 and 2

Racemic 1,1-dimethylethyl {[(cis)-4-hydroxy-1-(phenylmethyl)-3-piperidinyl]methyl}carbamate (5 g) was subjected to preparative HPLC on Chiralpak AD (77×240 mm column) eluting with 90:10:0.1 heptane:isopropanol:isopropylamine. This procedure gave the faster running enantiomer (Enantiomer 1, 2.2 g) in >98% ee and the slower running enantiomer (Enantiomer 2, 2.2 g) in 97% ee.

### (h) 1,1-Dimethylethyl {[(cis)-4-hydroxy-3-piperidinyl]methyl}carbamate Enantiomer

A solution of 1,1-dimethylethyl {[(cis)-4-hydroxy-1-(phenylmethyl)-3-piperidinyl]methyl}carbamate Enantiomer 1 (2.2 g, 6.8 mmol) in methanol (50 ml) was stirred under hydrogen and at room temperature in presence of 20% Palladium hydroxide (0.5 g) for 18 h. After filtration through kieselguhr, the methanol was removed under reduced pressure to afford the desired product (1.6 g, 100%).

MS (ES+) m/z 231 (MH⁺, 100%).

### (i) 1,1-Dimethylethyl [(1-{[(6R)-3-fluoro-8-oxo-5,6-dihydro-8H-[1,4]oxazino[2,3,4-de]-1,5-naphthyridin-6-yl]methyl}-4-hydroxy-3-piperidinyl)methyl]carbamate Enantiomer 1

[(6S)-3-Fluoro-8-oxo-5,6-dihydro-8H-[1,4]oxazino[2,3,4-de]-1,5-naphthyridin-6-yl]methyl methanesulfonate (for a preparation see Example 8(g)) (0.16 g, 0.51 mmol) was converted to the title compound on treatment with 1,1-dimethylethyl {[(cis)-4-hydroxy-3-piperidinyl]methyl}carbamate Enantiomer 1 (0.175 g, 0.76 mmol)) under the general conditions described for Example 7(e). After work-up and chromatography the desired product was obtained as a white foam (0.109 g).

MS (ES+), m/z 449 (MH⁺, 100%).

### (j) (6R)-6-{[3-(Aminomethyl)-4-hydroxy-1-piperidinyl]methyl}-3-fluoro-5,6-dihydro-8H-[1,4]oxazino[2,3,4-de]-1,5-naphthyridin-8-one

1,1-Dimethylethyl [(1-{[(6R)-3-fluoro-8-oxo-5,6-dihydro-8H-[1,4]oxazino[2,3,4-de]-1,5-naphthyridin-6-yl]methyl}-4-hydroxy-3-piperidinyl)methyl]carbamate Enantiomer 1 (0.109 g, 0.24 mmol) was treated with trifluoroacetic acid (2 mL) in DCM (10 mL) for 18 h at RT then evaporated to dryness. The residue was dissolved in methanol (50 mL) and Amberlyst A21 ion exchange resin (approx 10 g) was added and the mixture stirred for 1 h. The mixture was then filtered and the filtrate evaporated to dryness to give the title compound as a white foam (0.086 g).

MS (ES+), m/z 349 (MH⁺, 100%).

### (k) Title Compound

(6R)-6-{[3-(Aminomethyl)-4-hydroxy-1-piperidinyl]methyl}-3-fluoro-5,6-dihydro-8H-[1,4]oxazino[2,3,4-de]-1,5-naphthyridin-8-one (0.04 g, 0.11 mmol) was treated with (2,3-dihydro[1,4]dioxino[2,3-c]pyridine-7-carboxaldehyde (for a synthesis see WO2004058144, Example 2(c) or WO2003087098 Example 19(d)) (0.018 g, 0.11 mmol) under the general conditions of Example 8(j) omitting sodium acetate. The free base of the title compound was obtained as a colourless oil (0.024 g).

1H NMR (250 MHz, CDCl₃). δ 1.73-1.83 (2H, m), 1.88-1.98 (1H, m), 2.31-2.68 (7H, m), 2.77-2.92 (2H, m), 3.02-3.14 (1H, m), 3.69-3.83 (2H, m), 3.95-4.05 (1H, m), 4.08-4.17 (1H, m), 4.25-4.39 (4H, m), 4.89-5.03 (2H, m), 6.77 (1H, s), 6.83 (1H, d, J 9.7 Hz), 7.88 (1H, d, J 9.7 Hz), 8.11 (1H, s), 8.38 (1H, d, J 2 Hz).

MS (ES+) m/z 498 (MH⁺, 100%).

The free base of the title compound was dissolved in methanol (5 mL) and treated with excess 4M hydrogen chloride in 1,4-dioxane then evaporated to dryness to give the title compound as a white solid.

### Example 27

### (6R)-3-Fluoro-6-[(4-hydroxy-3-{[([1,3]oxathiolo[5,4-c]pyridin-6-ylmethyl)amino]methyl}-1-piperidinyl)methyl]-5,6-dihydro-8H-[1,4]oxazino[2,3,4-de]-1,5-naphthyridin-8-one dihydrochloride

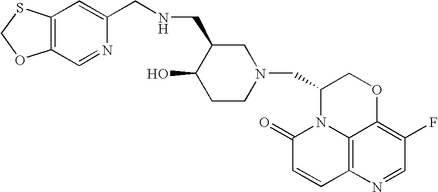

(6R)-6-{[3-(Aminomethyl)-4-hydroxy-1-piperidinyl]methyl}-3-fluoro-5,6-dihydro-8H-[1,4]oxazino[2,3,4-de]-1,5-naphthyridin-8-one (for a preparation see Example 26(j)) (0.04 g, 0.11 mmol) was treated with [1,3]oxathiolo[5,4-c]pyridine-6-carbaldehyde (0.018 g, 0.11 mmol) (for a synthesis see WO2004058144 Example 61) under the general conditions of Example 8(j) omitting sodium acetate. The free base of the title compound was obtained as a colourless oil (0.026 g).

1H NMR (250 MHz, CDCl3). δ 1.71-181 (2H, m), 1.90-2.02 (1H, m), 2.32-3.17 (10H, m), 3.71-3.89 (2H, m), 3.94-4.05 (1H, m), 4.08-4.18 (1H, m), 4.88-5.05 (2H, m), 5.75 (2H, s), 6.81 (1H, d, J 9.7 Hz), 7.14 (1H, s), 7.85 (1H, d, J 9.7 Hz), 8.01 (1H, s), 8.38 (1H, d, J 2 Hz).

MS (ES+) m/z 500 (MH⁺, 100%).

The free base of the title compound was dissolved in methanol (5 mL) and treated with excess 4M hydrogen chloride in 1,4-dioxane then evaporated to dryness to give the title compound as a white solid.

### Example 28

### 3-Fluoro-6-{[(3S,4S)-3-hydroxy-4-({[(3-oxo-3,4-dihydro-2H-pyrido[3,2-b][1,4]oxazin-6-yl)methyl]amino}methyl)-1-pyrrolidinyl]methyl}-5,6-dihydro-4H,8H-pyrido[3,2,1-de]-1,5-naphthyridin-8-one dihydrochloride

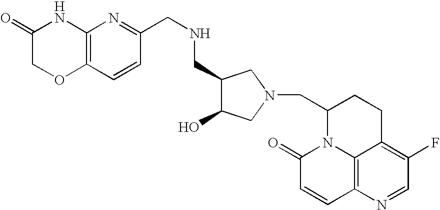

### (a) (3-Fluoro-8-oxo-5,6-dihydro-4H,8H-pyrido[3,2,1-de]-1,5-naphthyridin-6-yl)methyl methanesulfonate

3-Fluoro-6-(hydroxymethyl)-5,6-dihydro-4H,8H-pyrido[3,2,1-de]-1,5-naphthyridin-8-one (for a preparation see Example 15(f)) (1.4 g, 6 mmol) was converted to the title compound by the general method of Example 12(c) to give an off-white solid (1.84 g).

MS (ES+) m/z 313 (MH⁺, 100%).

### (b) Phenylmethyl ({(3S,4S)-1-[(3-fluoro-8-oxo-5,6-dihydro-4H,8H-pyrido[3,2,1-de]-1,5-naphthyridin-6-yl)methyl]-4-hydroxy-3-pyrrolidinyl}methyl)carbamate

(3-Fluoro-8-oxo-5,6-dihydro-4H,8H-pyrido[3,2,1-de]-1,5-naphthyridin-6-yl)methyl methanesulfonate (0.277 g, 0.88 mmol) was converted to the title compound on treatment with phenylmethyl {[(3R,4S)-4-hydroxy-3-pyrrolidinyl]methyl}carbamate (for a preparation see Example 21(a)) (0.256 g, 1 mmol) under the general conditions described for Example 7(e). After work-up and chromatography the desired product was obtained as a colourless oil (0.208 g).

MS (ES+), m/z 467 (MH⁺, 100%).

### (c) 6-{[(3S,4S)-3-(Aminomethyl)-4-hydroxy-1-pyrrolidinyl]methyl}-3-fluoro-5,6-dihydro-4H,8H-pyrido[3,2,1-de]-1,5-naphthyridin-8-one

Phenylmethyl ({(3S,4S)-1-[(3-fluoro-8-oxo-5,6-dihydro-4H,8H-pyrido[3,2,1-de]-1,5-naphthyridin-6-yl)methyl]-4-hydroxy-3-pyrrolidinyl}methyl)carbamate (0.208 g, 0.44 mmol) was dissolved in ethanol (30 mL) and hydrogenated at S.T.P over a 10% palladium on charcoal paste catalyst for 3 h. The catalyst was filtered off and the filtrate evaporated to dryness to give the title compound (0.155 g).

MS (ES+), m/z 333 (MH⁺, 80%).

### (d) Title Compound

6-{[(3S,4S)-3-(Aminomethyl)-4-hydroxy-1-pyrrolidinyl]methyl}-3-fluoro-5,6-dihydro-4H,8H-pyrido[3,2,1-de]-1,5-naphthyridin-8-one (0.07 g, 0.21 mmol) was treated with 3-oxo-3,4-dihydro-2H-pyrido[3,2-b][1,4]oxazine-6-carbaldehyde (0.037 g, 0.21 mmol) (for a synthesis see WO2003087098 Example 31(e)) under the general conditions of Example 8(j). The free base of the title compound was obtained as a colourless oil (0.046 g).

1H NMR (250 MHz, CDCl3) δ 1.71-1.91 (1H, m), 2.35-3.11 (13H, m), 3.68-3.88 (2H, m), 4.37-4.45 (1H, m), 4.64 (2H, d, J 2 Hz), 5.17-5.31 (1H, m), 6.81-6.95 (2H, m), 7.19 (1H, d, J 10 Hz), 7.87 (1H, d, J 10 Hz), 8.37 (1H, s).

MS (ES+) m/z 495 (MH⁺, 100%).

The free base of the title compound was dissolved in methanol (5 mL) and treated with excess 4M hydrogen chloride in 1,4-dioxane then evaporated to dryness to give the title compound as a white solid.

### Example 29

### 3-Fluoro-6-{[(3S,4S)-3-hydroxy-4-({[(2-oxo-2,3-dihydro-1H-pyrido[3,4-b][1,4]oxazin-7-yl)methyl]amino}methyl)-1-pyrrolidinyl]methyl}-5,6-dihydro-4H,8H-pyrido[3,2,1-de]-1,5-naphthyridin-8-one dihydrochloride salt

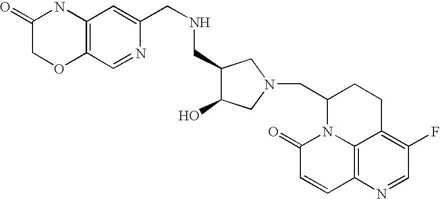

6-{[(3S,4S)-3-(Aminomethyl)-4-hydroxy-1-pyrrolidinyl]methyl}-3-fluoro-5,6-dihydro-4H,8H-pyrido[3,2,1-de]-1,5-naphthyridin-8-one (for a preparation see Example 28(c)) (0.065 g, 0.19 mmol) was treated with 2-oxo-2,3-dihydro-1H-pyrido[3,4-b][1,4]oxazine-7-carbaldehyde (0.035 g, 0.19 mmol) (for a synthesis see WO2003087098 Example 309(i)) under the general conditions of Example 8(j). The free base of the title compound was obtained as a colourless oil (0.028 g).

1H NMR (250 MHz, CDCl3) δ 1.75-1.91 (1H, m), 2.35-3.15 (13H, m), 3.72-3.91 (2H, m), 4.37-4.45 (1H, m), 4.64 (2H, s), 5.19-5.33 (1H, m), 6.83-7.02 (2H, m), 7.88 (1H, d, J 10 Hz), 8.18 (1H, d, J 4 Hz), 8.38 (1H, d, J 1.5 Hz).

MS (ES+) m/z 495 (MH⁺, 60%).

The free base of the title compound was dissolved in methanol (5 mL) and treated with excess 4M hydrogen chloride in 1,4-dioxane then evaporated to dryness to give the title compound as a white solid.

### Example 30

### 6-({(3R,4S)-4-[(2,3-dihydro[1,4]dioxino[2,3-c]pyridin-7-ylmethyl)amino]-3-hydroxy-1-piperidinyl}methyl)-3-fluoro-5,6-dihydro-4H,8H-pyrido[3,2,1-de]-1,5-naphthyridin-8-one dihydrochloride

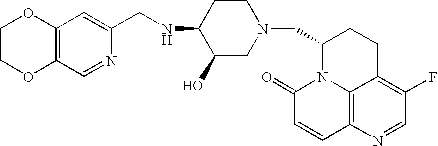

### (a) 1,1-Dimethylethyl {(3R,4S)-1-[(3-fluoro-8-oxo-5,6-dihydro-4H,8H-pyrido[3,2,1-de]-1,5-naphthyridin-6-yl)methyl]-3-hydroxy-4-piperidinyl}carbamate

(3-Fluoro-8-oxo-5,6-dihydro-4H,8H-pyrido[3,2,1-de]-1,5-naphthyridin-6-yl)methyl methanesulfonate (for a preparation see Example 28(a)) (0.45 g, 1.4 mmol) was converted to the title compound on treatment with cis-(3-hydroxy-piperidin-4-yl)-carbamic acid tert-butyl ester enantiomer 1 (for a synthesis see WO 2004/058144 Example 5(c)) (0.405 g, 1.9 mmol) under the general conditions described for Example 7(e). After work-up and chromatography the desired product was obtained as a pale yellow oil (0.42 g).

MS (ES+), m/z 433 (MH⁺, 100%).

### (b) 6-{[(3R,4S)-4-Amino-3-hydroxy-1-piperidinyl]methyl}-3-fluoro-5,6-dihydro-4H,8H-pyrido[3,2,1-de]-1,5-naphthyridin-8-one trifluoroacetate salt

1,1-Dimethylethyl {(3R,4S)-1-[(3-fluoro-8-oxo-5,6-dihydro-4H,8H-pyrido[3,2,1-de]-1,5-naphthyridin-6-yl)methyl]-3-hydroxy-4-piperidinyl}carbamate (0.42 g, 0.97 mmol) was treated with trifluoroacetic acid (5 mL) in DCM (20 mL) for 18 h at RT then evaporated to dryness. The residue was dissolved in methanol (50 mL) and Amberlyst A21 ion exchange resin (approx 15 g) was added and the mixture stirred for 1 h. The mixture was then filtered and the filtrate evaporated to dryness to give the title compound as a white solid (0.386 g).

MS (ES+), m/z 333 (MH⁺, 100%).

### (c) Title Compound

6-{[(3R,4S)-4-Amino-3-hydroxy-1-piperidinyl]methyl}-3-fluoro-5,6-dihydro-4H,8H-pyrido[3,2,1-de]-1,5-naphthyridin-8-one trifluoroacetate salt (0.096 g, 0.2 mmol) was treated with 2,3-dihydro[1,4]dioxino[2,3-c]pyridine- (0.081 g, 0.49 mmol) (for a synthesis see WO2004058144, Example 2(c) or WO2003087098 Example 19(d)) under the general conditions of Example 8(j). The free base of the title compound was obtained as a colourless oil (0.068 g).

1H NMR (250 MHz, CDCl3) δ 1.61-1.95 (3H, m), 2.09-2.89 (8H, m), 2.99-3.21 (2H, m), 3.78-3.95 (3H, m), 4.23-4.38 (4H, m), 5.17-5.37 (1H, m), 6.78-6.89 (2H, m), 7.84 (1H, dd, J 6 and 10 Hz), 8.09 (1H, t, J 3.5 Hz), 8.36 (1H, d, J 6 Hz).

MS (ES+) m/z 482 (MH+, 35%).

The free base of the title compound was dissolved in methanol (5 mL) and treated with excess 4M hydrogen chloride in 1,4-dioxane then evaporated to dryness to give the title compound as a white solid.

### Example 69

### (6R)-6-({4-[(2,3-dihydro[1,4]dioxino[2,3-c]pyridin-7-ylmethyl)amino]-1-piperidinyl}methyl)-3-fluoro-5,6-dihydro-8H-[1,4]thiazino[2,3,4-de]-1,5-naphthyridin-8-one dihydrochloride

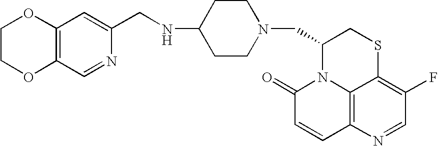

### (a) 8-[(1,1-Dimethylethyl)thio]-7-fluoro-2-(methyloxy)-1,5-naphthyridine

A mixture of 8-bromo-7-fluoro-2-(methyloxy)-1,5-naphthyridine (for a synthesis see WO2004058144, Example 53(g)) (2.57 g, 10 mmol) and (s)-(−)-tol-BINAP (620 mg, 10 mol %) in toluene (170 ml) was purged with argon, the flask evacuated and the procedure repeated three times. Palladium (II) acetate (220 mg) was added and the mixture stirred for 10 min. Solid sodium tert-butyl thyiolate (1.56 g) was added and the flask purged with argon. The reaction mixture was heated to 80° C. for 24 h, then refluxed for 2 days. The solvent was evaporated and the residue partitioned between water and ethyl acetate. The organic phase was washed with brine, dried (MgSO₄) and evaporated. The residue was chromatographed on silica eluting with 0-100% ethyl acetate in hexane to afford the title compound as a pale yellow oil (1.58 g, 59%).

MS (ES+), m/z 267 (MH⁺, 40%).

### (b) 3-Fluoro-6-(methyloxy)-1,5-naphthyridine-4-thiol

8-[(1,1-Dimethylethyl)thio]-7-fluoro-2-(methyloxy)-1,5-naphthyridine (0.1 g, 0.37 mmol) was dissolved in conc. HCl (1 ml) and the solution stirred at room temperature for 30 min, then evaporated to dryness. The residue was chromatographed on silica eluting with 1-10% 2M methanolic ammonia in DCM, to afford the title compound as yellow solid (0.034 g, 50%)

MS (ES+), m/z 211 (MH⁺, 100%).

### (c) 7-Fluoro-2-(methyloxy)-8-{[(2S)-2-oxiranylmethyl]thio}-1,5-naphthyridine

3-Fluoro-6-(methyloxy)-1,5-naphthyridine-4-thiol (0.45 g, 2.1 mmol) was dissolved in dry DMF (15 ml) and treated with potassium carbonate (0.29 g, 2.1 mmol) followed by S-(+)-glycidyl nosylate (0.55 g, 2.1 mmol), and the mixture stirred at room temperature overnight. The solvent was evaporated under reduced pressure and the residue dissolved in ethyl acetate and washed with saturated sodium bicarbonate, water, and brine. The organic phase dried (MgSO₄), filtered and evaporated. The residue was chromatographed on silica gel eluting with a gradient of 0-50% ethyl acetate in hexane to give the title compound as a white solid. (0.53 g, 93%)

MS (ES+), m/z 267 (MH⁺, 100%).

### (d) (6R)-3-Fluoro-6-(hydroxymethyl)-5,6-dihydro-8H-[1,4]thiazino[2,3,4-de]-1,5-naphthyridin-8-one

7-Fluoro-2-(methyloxy)-8-{[(2S)-2-oxiranylmethyl]thio}-1,5-naphthyridine (0.52 g, 1.95 mmol) was dissolved in dichloromethane (10 ml) and THF (10 ml). The solution was treated with ytterbium (III) trifluoromethanesulphonate (1.2 g, 1.95 mmol) and the mixture stirred at room temperature overnight. The reaction mixture was concentrated under reduced pressure. The residue was partitioned between 10% methanol in dichloromethane and saturated sodium hydrogen bicarbonate solution (50 ml). The aqueous phase extracted with 10% methanol in dichloromethane (3×50 ml). The organics combined and dried (MgSO₄) and concentrated under reduced pressure. The residue was chromatographed on silica gel eluting with a gradient of 0-10% methanol in dichloromethane to give the title compound as a white solid. (0.48 g, 98%)

MS (ES+), m/z 253 (MH⁺, 100%).

### (e) [(6R)-3-Fluoro-8-oxo-5,6-dihydro-8H-[1,4]thiazino[2,3,4-de]-1,5-naphthyridin-6-yl]methyl methanesulfonate

(6R)-3-Fluoro-6-(hydroxymethyl)-5,6-dihydro-8H-[1,4]thiazino[2,3,4-de]-1,5-naphthyridin-8-one. (0.47 g. 1.88 mmol) was suspended in dichloromethane, treated with triethylamine (0.3 ml, 2.26 mmol), and cooled in an ice bath. Methanesulfonyl chloride (0.17 ml, 2.26 mmol) was added and the cooling bath was removed. The reaction mixture was stirred at room temperature for 20 mins. The reaction mixture was washed with water, saturated aqueous sodium hydrogen carbonate solution and brine, dried over (MgSO₄), filtered and evaporated to give the title compound as a white solid (0.5 g, 81%)

MS (ES+), m/z 331 (MH⁺, 100%).

### (f) (6R)-6-[(4-Amino-1-piperidinyl)methyl]-3-fluoro-5,6-dihydro-8H-[1,4]thiazino[2,3,4-de]-1,5-naphthyridin-8-one dihydrochloride

[(6R)-3-Fluoro-8-oxo-5,6-dihydro-8H-[1,4]thiazino[2,3,4-de]-1,5-naphthyridin-6-yl]methyl methanesulfonate (0.5 g, 1.5 mmol) was dissolved in dry acetonitrile (25 ml), treated with dry pyridine (0.24 ml 3 mmol) followed by 1,1-dimethyethyl 4-piperidinylcarbamate (0.6 g, 3 mmol). The mixture was heated to 70° C. for 18 hr. The mixture was cooled, diluted with ethyl acetate (30 ml), washed with water and brine. The organic phase was dried over (MgSO₄), filtered and evaporated. The residue was chromatographed on silica gel eluting with a gradient of 0-10% 2M methanolic ammonia in dichloromethane to give 1,1-dimethylethyl (1-{[(6R)-3-fluoro-8-oxo-5,6-dihydro-8H-[1,4]thiazino[2,3,4-de]-1,5-naphthyridin-6-yl]methyl}-4-piperidinyl)carbamate as a pale yellow solid. (70 mg, 11%). This was dissolved in dry DCM (4 ml) treated with 4 M HCl (0.16 ml 3.0 mmol) in 1,4-dioxane. The reaction mixture was stirred for 10 min at room temperature. Further 4 M HCl (0.16 ml 3.0 mmol) was added dropwise and the reaction stirred for a further 15 min. The solvent was evaporated to obtain the title compound as a free flowing white solid. (75 mg, containing 0.16 mmol)

MS (ES+), m/z 335 (MH⁺, 100%).

### (g) Title Compound

(6R)-6-[(4-Amino-1-piperidinyl)methyl]-3-fluoro-5,6-dihydro-8H-[1,4]thiazino[2,3,4-de]-1,5-naphthyridin-8-one dihydrochloride. (37 mg, containing 0.08 mmol) was dissolved in methanol (4 ml) and acetic acid (0.04 ml) and treated with sodium acetate anhydrous (21 mg, 2.5 mmol) and 2,3-dihydro[1,4]dioxino[2,3-c]pyridine-7-carbaldehyde (for a synthesis see WO2004058144, Example 2(c) or WO2003087098 Example 19(d)) (20 mg, 0.12 mmol). The reaction mixture was left stirring at room temperature for 20 min. Sodium cyanoborohydride (6.22 mg, 0.1 mmol) was added and the reaction stirred at room temperature overnight. The reaction mixture was applied to a SCX cartridge (2 g) and eluted with methanol (10 ml). and 0.2 M ammonia in methanol (10 ml). Fractions containing the compound were combined and evaporated. The residue was chromatographed on silica gel eluting with a gradient of 0-20% 2M methanolic ammonia in dichloromethane to give the free base of the title compound as a pale yellow solid. (18.5 mg, 39%)

H NMR (400 MHz, CDCl₃) δ 1.71-1.95 (2H, m), 2.03-2.15 (2H, m), 2.24-2.33 (2H, m), 2.49-2.56 (1H, m), 2.72-2.78 (1H, m), 2.84 (2H, m), 3.07-3.16 (1H, m), 3.19-3.23 (1H, m), 3.43-3.53 (4H, m), 4.26-4.34 (4H, m), 5.57-5.60 (1H, m), 6.79-6.84 (2H, m), 7.86 (1H, d, J=8 Hz), 8.10 (1H, s), 8.29 (1H, s).

MS (ES+), m/z 484 (MH⁺, 100%), m/z 633 (MH⁺, 50%).

The free base of the title compound (18.5 mg, 0.038 mmol) was dissolved in methanol (2 ml) and treated with 1M HCl in diethyl ether (0.11 ml, 3 eqs.). The resulting mixture was evaporated to dryness to afford the di-HCl salt as a yellow solid.

MS (ES+), m/z 484, 532 (MH⁺, 40%).

### Example 70

### (6R)-6-({4-[(6,7-dihydro[1,4]dioxino[2,3-c]pyridazin-3-ylmethyl)amino]-1-piperidinyl}methyl)-3-fluoro-5,6-dihydro-8H-[1,4]thiazino[2,3,4-de]-1,5-naphthyridin-8-one fumarate

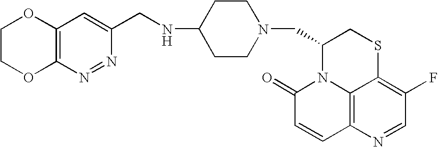

(6R)-6-[(4-amino-1-piperidinyl)methyl]-3-fluoro-5,6-dihydro-8H-[1,4]thiazino[2,3,4-de]-1,5-naphthyridin-8-one dihydrochloride (37 mg, containing 0.08 mmol) was dissolved in methanol (4 ml) and acetic acid (0.04 ml) and treated with sodium acetate (21 mg, 2.5 mmol) and 2,3-dihydro[1,4]dioxino[2,3-c]pyridine-7-carbaldehyde (for a preparation see Example 3(e)) (16 mg, 0.1 mmol). The reaction mixture was left stirring at room temperature for 20 min. Sodium cyanoborohydride (6.22 mg, 0.1 mmol) was added and the reaction left stirring at room temperature overnight. The reaction mixture was applied to an SCX cartridge (2 g) and eluted with methanol (10 ml). and 0.2 M ammonia in methanol (10 ml). Fractions containing the compound were combined and evaporated. The residue was chromatographed on silica gel eluting with a gradient of 0-20% 2M methanolic ammonia in dichloromethane to give the free base of the title compound as a pale yellow solid. (6 mg, 13%).

¹HNMR (400 MHz, CDCl₃) δ 1.89-1.90 (2H, m), 2.14-2.28 (1H, m), 2.30-2.34 (2H, m), 2.50-2.53 (2H, m), 2.72-2.78 (2H, m), 3.02-3.04 (1H, m), 3.07-3.09 (1H, m), 3.64-3.69 (1H, m), 4.36-3.40 (4H, m), 4.50-4.56 (4H, m), 5.59-5.61 (1H, m), 6.85 (2H, d, J=8 Hz), 7.02 (1H, s), 7.85 (1H, d, J=8 Hz), 8.29 (1H, s).

MS (ES+), m/z 485 (MH⁺, 100%).

The free base was dissolved in methanol (1 ml) 0.1 M solution of fumaric acid in methanol (0.1 ml, 1 equivalent) added dropwise and the reaction stirred for 5 min. and solvent evaporated to give the title compound.

### Example 71

### 10-Chloro-3-({4-[(2,3-dihydro[1,4]dioxino[2,3-c]pyridin-7-ylmethyl)amino]-1-piperidinyl}methyl)-2,3-dihydro-5H-[1,4]oxazino[4,3,2-de]quinoxalin-5-one dihydrochloride (Enantiomer 1)

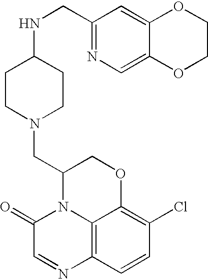

### (a) 7-Chloro-2-(methyloxy)-8-{[(2S)-2-oxiranylmethyl]oxy}quinoxaline

6-Chloro-3-(methyloxy)-5-quinoxalinol (for a synthesis see WO2006021448 Example 93(f)). (1.0 g, 4.7 mmol) was dissolved in dry DMF (30 ml), treated with sodium hydride (60% dispersion in oil) (0.18 g, 4.7 mmol) and left stirring at room temperature for 30 min. S-(+)-Glycidyl nosylate (1.46 g, 5.64 mmol) was added and the reaction mixture stirred under an argon atmosphere at room temperature overnight. The reaction mixture was concentrated under reduced pressure. The residue was partitioned between ethyl acetate and water, and the aqueous layer extracted with ethyl acetate (100 ml×4), organics combined, dried (MgSO₄), filtered and evaporated. The residue was chromatographed on silica gel eluting with a gradient of 0-40% ethyl acetate in hexane to give the title compound as an off-white solid (0.45 g, 38%).

MS (ES+), m/z 267 (MH⁺, 100%).

### (b) 1,1-Dimethylethyl [1-(3-{[6-chloro-3-(methyloxy)-5-quinoxalinyl]oxy}-2-hydroxypropyl)-4-piperidinyl](2,3-dihydro[1,4]dioxino[2,3-c]pyridin-7-ylmethyl)carbamate (Enantiomer 1)

7-Chloro-2-(methyloxy)-8-{[(2S)-2-oxiranylmethyl]oxy}quinoxaline (100 mg, 0.38 mmol) was dissolved in acetonitrile (10 ml) followed by 1,1-dimethylethyl (2,3-dihydro[1,4]dioxino[2,3-c]pyridin-7-ylmethyl)4-piperidinylcarbamate (for a synthesis see WO2004058144 Example 99(h)) (131 mg, 0.38 mmol). The reaction mixture was heated at 80° C. for 72 hr. The solvent was concentrated under reduced pressure and the residue was chromatographed on silica gel eluting with a gradient of 0-10% 2M methanolic ammonia in dichloromethane to give the title compound as a pale yellow oil. (135 mg, 58%).

MS (ES+), m/z 616.5 (MH⁺, 100%).

### (c) 1,1-Dimethylethyl (1-{[10-chloro-5-oxo-2,3-dihydro-5H-[1,4]oxazino[4,3,2-de]quinoxalin-3-yl]methyl}-4-piperidinyl)(2,3-dihydro[1,4]dioxino[2,3-c]pyridin-7-ylmethyl)carbamate (Enantiomer 1)

1,1-Dimethylethyl [1-(3-{[6-chloro-3-(methyloxy)-5-quinoxalinyl]oxy}-2-hydroxypropyl)-4-piperidinyl](2,3-dihydro[1,4]dioxino[2,3-c]pyridin-7-ylmethyl)carbamate (135 mg, 0.22 mmol) was dissolved in chloroform (15 ml), diisopropylethylamine (0.041 ml, 0.24 mmol) added followed by methanesulfonic anhydride (42 mg, 0.24 mmol). The reaction mixture heated at 60° C. overnight. The reaction mixture was treated with a further 1 equivalent of diisopropylethylamine and methanesulfonic anhydride and left heating at 60° C. overnight. The reaction mixture was washed with saturated sodium bicarbonate, water and brine, dried (MgSO₄) and evaporated. The residue was chromatographed on silica gel eluting with a gradient of 0-10% 2M methanolic ammonia in dichloromethane to give the title compound as a white foam. (55 mg, 43%).

MS (ES+), m/z 584 (MH⁺, 50%), m/z 616 (MH⁺, 30%).

### (d) Title Compound

1,1-Dimethylethyl (1-{[10-chloro-5-oxo-2,3-dihydro-5H-[1,4]oxazino[4,3,2-de]quinoxalin-3-yl]methyl}-4-piperidinyl)(2,3-dihydro[1,4]dioxino[2,3-c]pyridin-7-ylmethyl)carbamate Enantiomer 1 (55 mg, 0.09 mmol) was dissolved in dichloromethane (4 ml) and treated with trifluoroacetic acid (2 ml). The reaction mixture was left stirring at room temperature for 30 min. The solvent was concentrated under reduced pressure. The residue was chromatographed on silica gel eluting with a gradient of 0-10% 2M methanolic ammonia in dichloromethane to give the free base of the title compound as a brown oil (10 mg, 23%).

¹H NMR (400 MHz, CDCl₃) δ 1.88-1.94 (2H, m), 2.16-2.27 (2H, m), 2.56-2.67 (2H, m), 2.77-304 (6H, m), 3.20-3.26 (2H, m), 3.51-3.54 (1H, m), 3.77-3.80 (1H, m), 4.91-4.97 (4H, m), 5.42-5.48 (1H, m) 7.44 (1H, s), 7.71 (1H, d J=8 Hz), 7.95 (1H, d, J=8 Hz), 8.71 (1H, s) 8.99 (1H, s).

MS (ES+), m/z 484 (MH⁺, 100%).

The free base was dissolved in methanol, 1 M HCl in diethyl ether added (2 equivalents). The solvent was evaporated to give the title compound as a yellow solid.

### Example 72

### 10-Chloro-3-({4-[(2,3-dihydro[1,4]dioxino[2,3-c]pyridin-7-ylmethyl)amino]-1-piperidinyl}methyl)-2,3-dihydro-5H-[1,4]oxazino[4,3,2-de]quinoxalin-5-one dihydrochloride (Enantiomer 2)

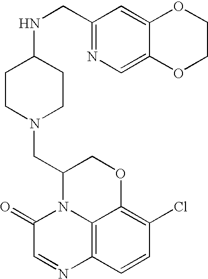

### (a) 7-Chloro-2-(methyloxy)-8-{[(2R)-2-oxiranylmethyl]oxy}quinoxaline

6-Chloro-3-(methyloxy)-5-quinoxalinol. (for a synthesis see WO2006021448 Example 93(f)) (0.45 g, 2.0 mmol) was dissolved in dry DMF (10 ml), treated with sodium hydride (60% dispersion in oil) (80 mg, 2.0 mmol) and left stirring at room temperature for 30 min. R-(−)-Glycidyl nosylate (0.62 g, 2.4 mmol) was added and the reaction mixture left stirring under argon atmosphere at room temperature overnight. The reaction mixture was concentrated under reduced pressure. The residue was partitioned between ethyl acetate and water. The aqueous layer extracted with ethyl acetate (50 ml×4), organics combined, dried (MgSO₄), filtered and evaporated. The residue was chromatographed on silica gel eluting with a gradient 0-40% ethyl acetate in hexane. to give the titled compound as an off-white solid (0.45 g, 85%).

MS (ES+), m/z 267 (MH⁺, 100%).

### (b) 1,1-Dimethylethyl [1-(3-{[6-chloro-3-(methyloxy)-5-quinoxalinyl]oxy}-2-hydroxypropyl)-4-piperidinyl]carbamate (Enantiomer 2)

7-Chloro-2-(methyloxy)-8-{[(2R)-2-oxiranylmethyl]oxy}quinoxaline (0.45 g, 1.70 mmol) was dissolved in acetonitrile (40 ml) followed by 1,1-dimethylethyl 4-piperidinylcarbamate (0.34 g, 1.70 mmol) The reaction mixture was heated at 80° C. for 72 hr. The solvent was concentrated under reduced pressure and the residue was chromatographed on silica gel eluting with a gradient of 0-10% 2M methanolic ammonia in dichloromethane to give the title compound as a pale yellow oil (0.59 g, 75%).

MS (ES+), m/z 467 (MH⁺, 100%).

### (c) 1,1-Dimethylethyl (1-{[10-chloro-5-oxo-2,3-dihydro-5H-[1,4]oxazino[4,3,2-de]quinoxalin-3-yl]methyl}-4-piperidinyl)carbamate. (Enantiomer 2)

1,1-Dimethylethyl [1-(3-{[6-chloro-3-(methyloxy)-5-quinoxalinyl]oxy}-2-hydroxypropyl)-4-piperidinyl]carbamate. (0.59 g, 1.27 mmol) was dissolved in 1,2 dichloroethane (20 ml), di-isopropylethylamine (0.238 ml, 1.40 mmol) added followed by methanesulfonic anhydride (244 mgs, 1.40 mmol). The reaction mixture heated at 60° C. overnight. The reaction mixture was treated with a further 1 equivalent of diisopropylethylamine and methanesulfonic anhydride and left heating at 60° C. for 2 hr. The reaction mixture was washed with saturated sodium hydrogen bicarbonate, water, brine, and dried over (MgSO₄) and evaporated. The residue was chromatographed on silica gel eluting with a gradient of 0-10% 2M methanolic ammonia in dichloromethane to give the title compound as a brown oil. (260 mg, 47%).

MS (ES+), m/z 435 (MH⁺, 100%)

### (d) 3-[(4-Amino-1-piperidinyl)methyl]-10-chloro-2,3-dihydro-5H-[1,4]oxazino[4,3,2-de]quinoxalin-5-one. (Enantiomer 2)

1,1-Dimethylethyl (1-{[10-chloro-5-oxo-2,3-dihydro-5H-[1,4]oxazino[4,3,2-de]quinoxalin-3-yl]methyl}-4-piperidinyl)carbamate (260 mg, 0.60 mmol) was dissolved in dichloromethane. (20 ml) and treated with trifluoroacetic acid (10 ml). The reaction mixture was left stirring at room temperature for 30 min. The solvent was concentrated under reduced pressure. The residue was chromatographed on silica gel eluting with a gradient of 0-20% 2M methanolic ammonia in dichloromethane to give the title compound as a brown oil. (150 mg, 75%).

MS (ES+), m/z 335 (MH⁺, 100%).

### (e) 10-Chloro-3-({4-[(2,3-dihydro[1,4]dioxino[2,3-c]pyridin-7-ylmethyl)amino]-1-piperidinyl}methyl)-2,3,6,7-tetrahydro-5H-[1,4]oxazino[4,3,2-de]quinoxalin-5-one (Enantiomer 2)

3-[(4-Amino-1-piperidinyl)methyl]-10-chloro-2,3-dihydro-5H-[1,4]oxazino[4,3,2-de]quinoxalin-5-one Enantiomer 2 (150 mgs, 0.50 mmol) and 2,3-dihydro[1,4]dioxino[2,3-c]pyridine-7-carbaldehyde (for a synthesis see WO2004058144, Example 2(c) or WO2003087098 Example 19(d)) (0.83 mg, 0.50 mmol) in methanol (20 ml) and acetic acid (0.2 ml) was treated with (polystyrylmethyl)trimethylammonium cyanoborohydride 4.0 mmol/gm (0.5 g, 2 mmol) and stirred at room temperature overnight The mixture was then filtered and the filtrate was evaporated to dryness. Chromatography on silica gel eluting with a gradient of 0-10% 2M ammonia/methanol in DCM gave the title compound as a yellow oil, a free base (80 mg, 33%).

MS (ES+), m/z 486 (MH⁺, 100%).

### (f) Title Compound

10-Chloro-3-({4-[(2,3-dihydro[1,4]dioxino[2,3-c]pyridin-7-ylmethyl)amino]-1-piperidinyl}methyl)-2,3,6,7-tetrahydro-5H-[1,4]oxazino[4,3,2-de]quinoxalin-5-one (Enantiomer 2) (70 mg, 0.14 mmol) was dissolved in dichloromethane and treated with mangansese dioxide (104 mg, 1.2 mmol). The reaction mixture left stirring at room temperature for 72 hr. The reaction mixture filtered through kieselguhr, filtrate concentrated under reduced pressure and the residue chromatographed on silica gel eluting with a gradient of 0-10% 2M ammonia/methanol in DCM gave the free base of the title compound as a yellow oil. (15 mg, 21%).

¹HNMR (400 MHz, CDCl₃) δ 1.42-1.52 (4H, m), 1.86-1.93 (2H, m), 2.17-2.20 (1H, m), 2.25-2.31 (1H, m), 2.40-2.49 (1H, m), 2.52-2.62 (2H, m), 2.84-2.87 (1H, m), 3.10-3.13 (1H, m), 3.5 (1H, s), 3.79 (2H, s) 3.99-4.02 (1H, m) 4.27-4.37 (4H, m) 4.79-4.80 (1H, m), 6.82 (1H s) 7.07 (1H, d J=8 Hz), 7.29 (1H, d, J=12 Hz), 8.10 (1H, s), 8.34 (1H, s)

MS (ES+), m/z 484 (MH⁺, 100%).

### HCl Salt Formation:

The free base was dissolved in methanol, 1 M HCl in diethyl ether added (3 equivalents). The solvent was evaporated to give the title compound as a yellow solid.

### Example 73

### (6R)-6-({4-[(2,3-dihydro[1,4]dioxino[2,3-c]pyridin-7-ylmethyl)(methyl)amino]-1-piperidinyl}methyl)-3-fluoro-5,6-dihydro-8H-[1,4]oxazino[2,3,4-de]-1,5-naphthyridin-8-one

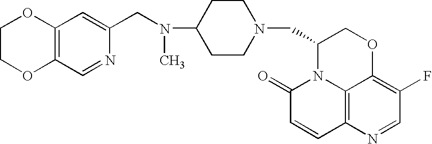

1,1-Dimethylethyl (1-{[(6R)-3-fluoro-8-oxo-5,6-dihydro-8H-[1,4]oxazino[2,3,4-de]-1,5-naphthyridin-6-yl]methyl}-4-piperidinyl)carbamate (for a synthesis, see Example 8(h) (207 mg, 0.52 mmol) was dissolved in dichloromethane (5 ml) and TFA (5 ml) and the mixture stirred at room temperature for 20 min. The solvent was evaporated under reduced pressure. The crude deprotected amine was dissolved in 10% methanol in dichloromethane (30 ml) and treated with carbonate resin (Argonaut) (1.0 g, 2/69 mmol) and stirred at room temperature for 1 h, filtered and evaporated. The procedure was repeated twice more using fresh carbonate resin each time, until ¹⁹F nmr indicated that TFA had been removed, to give 120 mg (0.38 mmol) of deprotected amine. This was reacted with 2,3-dihydro[1,4]dioxino[2,3-d]pyridine-7-carbaldehyde (66 mg, 0.4 mmol) (for a synthesis see WO2004058144, Example 2(c) or WO2003087098 Example 19(d)) under the general conditions described for Example 8 (j) to afford the title compound (62 mg, 37%).

MS (ES+), m/z 482 (MH⁺, 40%).

### Example 74

### (6S)-6-({4-[(2,3-dihydro[1,4]dioxino[2,3-c]pyridin-7-ylmethyl)amino]-1-piperidinyl}methyl)-3-fluoro-5,6-dihydro-8H-[1,4]oxazino[2,3,4-de]-1,5-naphthyridin-8-one dihydrochloride

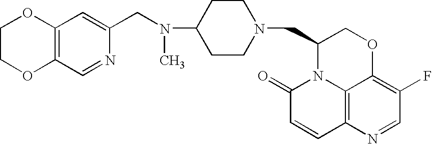

### (a) 7-Fluoro-2-(methyloxy)-8-{[(2R)-2-oxiranylmethyl]oxy}-1,5-naphthyridine

3-Fluoro-6-(methyloxy)-1,5-naphthyridin-4-ol (for a preparation, see Example 8(d)) (5.82 g, 30 mmol) was reacted with R-(−) glycidyl nosylate (9.3 g, 30 mmol) under the general conditions described for Example 8(e). After work-up and chromatography under similar conditions, the desired compound was obtained as a white solid containing approx 25% nitrobenzene sulfonate residues (4.5 g).

MS (ES+), m/z 251 (MH⁺, 100%).

### (b) (6S)-3-Fluoro-6-(hydroxymethyl)-5,6-dihydro-8H-[1,4]oxazino[2,3,4-de]-1,5-naphthyridin-8-one

7-Fluoro-2-(methyloxy)-8-{[(2R)-2-oxiranylmethyl]oxy}-1,5-naphthyridine (500 mg, containing approx. 1.48 mmol) was treated with ytterbium (III) trifluoromethanesulphonate (1.2 g, 1.93 mmol) under the general conditions described for Example 8 (f) to afford the title compound as a white solid (320 mg, 91%).

MS (ES+), m/z 237 (MH⁺, 100%).

### (c) [(6R)-3-Fluoro-8-oxo-5,6-dihydro-8H-[1,4]oxazino[2,3,4-de]-1,5-naphthyridin-6-yl]methyl methanesulfonate

(6S)-3-fluoro-6-(hydroxymethyl)-5,6-dihydro-8H-[1,4]oxazino[2,3,4-de]-1,5-naphthyridin-8-one (236 mg, 1.0 mmol) was converted to the mesylate under the general conditions described for Example 7(d). The product was obtained as a pale yellow solid (266 mg, 80%) and used without further purification.

MS (ES+), m/z 315 (MH⁺, 100%).

### (d) 1,1-Dimethylethyl (1-{[(6S)-3-fluoro-8-oxo-5,6-dihydro-8H-[1,4]oxazino[2,3,4-de]-1,5-naphthyridin-6-yl]methyl}-4-piperidinyl)carbamate

[(6R)-3-Fluoro-8-oxo-5,6-dihydro-8H-[1,4]oxazino[2,3,4-de]-1,5-naphthyridin-6-yl]methyl methanesulfonate (265 mg, 0.8 mmol) was reacted with 1,1-dimethylethyl 4-piperidinylcarbamate (400 mg, 2.0 mmol) under the general conditions described for Example 7(e). After work-up, the residue was chromatographed on silica gel eluting with 0-10% 2M methanolic ammonia in dichloromethane. Product-containing fractions were combined and evaporated to a colourless oil (210 mg, 50%).

MS (ES+), m/z 419 (MH⁺, 100%).

### (e) (6S)-6-[(4-Amino-1-piperidinyl)methyl]-3-fluoro-5,6-dihydro-8H-[1,4]oxazino[2,3,4-de]-1,5-naphthyridin-8-one dihydrochloride

1,1-Dimethylethyl (1-{[(6S)-3-fluoro-8-oxo-5,6-dihydro-8H-[1,4]oxazino[2,3,4-de]-1,5-naphthyridin-6-yl]methyl}-4-piperidinyl)carbamate (0.21 g, 0.5 mmol) was dissolved in dichloromethane (10 ml) and treated dropwise with a solution of 4M HCl in 1,4-dioxane (0.4 ml, 1.6 mmol) under the general conditions described for Example 8(i) to yield the title compound as a white free-flowing solid (0.236 g >100%). This was assumed to be the hydrated dihydrochloride salt.

MS (ES+), m/z 319 (MH⁺, 100%).

### (f) Title Compound

(6S)-6-[(4-Amino-1-piperidinyl)methyl]-3-fluoro-5,6-dihydro-8H-[1,4]oxazino[2,3,4-de]-1,5-naphthyridin-8-one dihydrochloride (0.236 g, containing 0.5 mmol of assumed di-hydrochloride) converted to the free base of the title compound by reaction with 2,3-dihydro[1,4]dioxino[2,3-c]pyridine-7-carbaldehyde (83 mg, 0.5 mmol) (for a synthesis see WO2004058144, Example 2(c) or WO2003087098 Example 19(d)) under the general conditions of Example 8(j) to give as a white foam (77 mg, 33%).

MS (ES+), m/z 468 (MH⁺, 50%), 490 (MNa+, 10%).

The free base (77 mg) was dissolved in methanol (2 ml) and treated with 1M HCl in methanol (0.33 ml, 2 equiv). The solvent was evaporated to give the title compound as an off-white solid.

Chiral hplc (ChiralpakAD 4.6×250 mm, 10 um, eluting with 100% ethanol) showed this material to be in >99.6% ee.

### Example 75

### (6S)-6-({4-[(6,7-dihydro[1,4]dioxino[2,3-c]pyridazin-3-ylmethyl)amino]-1-piperidinyl}methyl)-3-fluoro-5,6-dihydro-8H-[1,4]oxazino[2,3,4-de]-1,5-naphthyridin-8-one

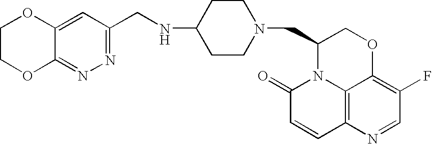

(6S)-6-[(4-Amino-1-piperidinyl)methyl]-3-fluoro-5,6-dihydro-8H-[1,4]oxazino[2,3,4-de]-1,5-naphthyridin-8-one dihydrochloride (0.189 g, 0.5 mmol) was reacted with 6,7-dihydro[1,4]dioxino[2,3-c]pyridazine-3-carbaldehyde (for a preparation see Example 3(e)) (0.083 g, 0.5 mmole) under the general conditions of Example 69(g) to afford the free base of the title compound as a white solid (0.095 g, 40%).

MS (ES+), m/z 469 (MH⁺, 100%), 491 (MNa+, 20%).

### Example 76

### (6S)-3-fluoro-6-({4-[([1,3]oxathiolo[5,4-c]pyridin-6-ylmethyl)amino]-1-piperidinyl}methyl)-5,6-dihydro-8H-[1,4]oxazino[2,3,4-de]-1,5-naphthyridin-8-one

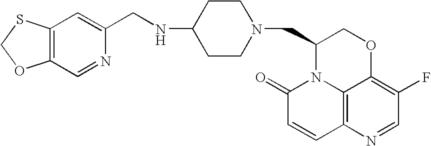

(6S)-6-[(4-Amino-1-piperidinyl)methyl]-3-fluoro-5,6-dihydro-8H-[1,4]oxazino[2,3,4-de]-1,5-naphthyridin-8-one dihydro chloride (0.189 g, 0.5 mmol) was reacted with [1,3]oxathiolo[5,4-c]pyridine-6-carbaldehyde (0.084 g, 0.5 mmole) (for a synthesis see WO2004058144 Example 61) under the general conditions of Example 69(g) to afford the free base of the title compound as a white solid (0.090 g, 38%).

MS (ES+), m/z 470 (MH⁺, 100%)

### Example 77

### (6R)-6-[(3-{[(2,3-dihydro[1,4]dioxino[2,3-c]pyridin-7-ylmethyl)amino]methyl}-4-hydroxy-1-piperidinyl)methyl]-3-fluoro-5,6-dihydro-8H-[1,4]oxazino[2,3,4-de]-1,5-naphthyridin-8-one dihydrochloride

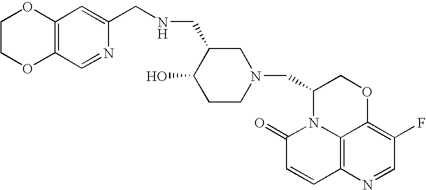

### (a) 1,1-Dimethylethyl {[(cis)-4-hydroxy-3-piperidinyl]methyl}carbamate Enantiomer 2

A solution of 1,1-dimethylethyl {[(cis)-4-hydroxy-1-(phenylmethyl)-3-piperidinyl]methyl}carbamate Enantiomer 2 (Example 26(g)) (2.2 g, 6.8 mmol) in methanol (50 ml) was stirred under hydrogen at room temperature in presence of 20% Palladium hydroxide (0.5 g) for 18 h. After filtration through kieselguhr, the methanol was removed under reduced pressure to afford the desired product (1.6 g, 100%).

MS (ES+) m/z 231 (MH+, 100%).

### (b) 1,1-Dimethylethyl [(1-{[(6R)-3-fluoro-8-oxo-5,6-dihydro-8H-[1,4]oxazino[2,3,4-de]-1,5-naphthyridin-6-yl]methyl}-4-hydroxy-3-piperidinyl)methyl]carbamate

[(6S)-3-Fluoro-8-oxo-5,6-dihydro-8H-[1,4]oxazino[2,3,4-de]-1,5-naphthyridin-6-yl]methyl methanesulfonate (for a preparation see Example 8(g)) (0.16 g, 0.51 mmol) was converted to the title compound on treatment with 1,1-dimethylethyl {[(cis)-4-hydroxy-3-piperidinyl]methyl}carbamate Enantiomer 2 (0.175 g, 0.76 mmol)) under the general conditions described for Example 7(e). After work-up and chromatography the desired product was obtained as a colourless oil (0.102 g).

MS (ES+), m/z 449 (MH⁺, 100%).

### (c) (6R)-6-{[3-(Aminomethyl)-4-hydroxy-1-piperidinyl]methyl}-3-fluoro-5,6-dihydro-8H-[1,4]oxazino[2,3,4-de]-1,5-naphthyridin-8-one trifluoroacetate salt

1,1-Dimethylethyl [(1-{[(6R)-3-fluoro-8-oxo-5,6-dihydro-8H-[1,4]oxazino[2,3,4-de]-1,5-naphthyridin-6-yl]methyl}-4-hydroxy-3-piperidinyl)methyl]carbamate (0.102 g, 0.22 mmol) was treated with trifluoroacetic acid (3 mL) in DCM (10 mL) for 18 h at RT then evaporated to dryness. The residue was dissolved in methanol (30 mL) and Amberlyst A21 ion exchange resin (approx 5 g) was added and the mixture stirred for 30 mins. The mixture was then filtered and the filtrate evaporated to dryness to give the title compound as a colourless oil (0.102 g).

MS (ES+), m/z 349 (MH⁺, 100%).

### (d) Title Compound

(6R)-6-{[3-(Aminomethyl)-4-hydroxy-1-piperidinyl]methyl}-3-fluoro-5,6-dihydro-8H-[1,4]oxazino[2,3,4-de]-1,5-naphthyridin-8-one trifluoroacetate salt (0.05 g, 0.11 mmol) was treated with (2,3-dihydro[1,4]dioxino[2,3-c]pyridine-7-carboxaldehyde (for a synthesis see WO2004058144, Example 2(c) or WO2003087098 Example 19(d)) (0.017 g, 0.11 mmol) under the general conditions of Example 8(j). The free base of the title compound was obtained as a colourless oil (0.01 g).

MS (ES+) m/z 498 (MH⁺, 35%).

The free base of the title compound was dissolved in methanol (3 mL) and treated with excess 4M hydrogen chloride in 1,4-dioxane then evaporated to dryness to give the title compound as a white solid.

### Example 78

### (6R)-6-[(3-{[(3,4-dihydro-2H-pyrano[2,3-c]pyridin-6-ylmethyl)amino]methyl}-4-hydroxy-1-piperidinyl)methyl]-3-fluoro-5,6-dihydro-8H-[1,4]oxazino[2,3,4-de]-1,5-naphthyridin-8-one dihydrochloride salt

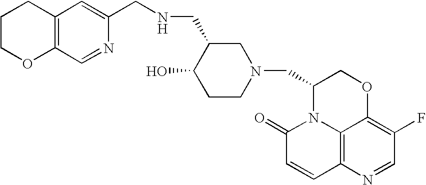

(6R)-6-{[3-(Aminomethyl)-4-hydroxy-1-piperidinyl]methyl}-3-fluoro-5,6-dihydro-8H-[1,4]oxazino[2,3,4-de]-1,5-naphthyridin-8-one trifluoroacetate salt (for a preparation see Example 77(c)) (0.05 g, 0.11 mmol) was treated with 3,4-dihydro-2H-pyrano[2,3-c]pyridine-6-carbaldehyde (for a synthesis see WO2004058144 Example 126(e)) (0.017 g, 0.11 mmol) under the general conditions of Example 8(j). The free base of the title compound was obtained as a colourless oil (0.02 g). MS (ES+) m/z 496 (MH⁺, 80%).

The free base of the title compound was dissolved in methanol (3 mL) and treated with excess 4M hydrogen chloride in 1,4-dioxane then evaporated to dryness to give the title compound as a white solid.

### Example 81

### (6R)-6-{[4-[(2,3-Dihydro[1,4]dioxino[2,3-c]pyridin-7-ylmethyl)amino]-4-(hydroxymethyl)-1-piperidinyl]methyl}-3-fluoro-5,6-dihydro-8H-[1,4]oxazino[2,3,4-de]-1,5-naphthyridin-8-one dihydrochloride

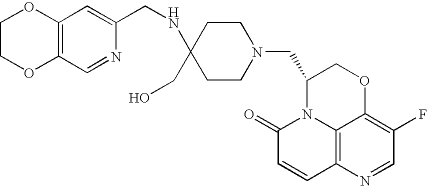

### (a) 1-(1,1-Dimethylethyl) 4-methyl 4-[(2,3-dihydro[1,4]dioxino[2,3-c]pyridin-7-ylmethyl)amino]-1,4-piperidinedicarboxylate

To a solution of 1-(1,1-dimethylethyl) 4-methyl 4-amino-1,4-piperidinedicarboxylate (methyl 4-amino 1-tert-butoxycarbonylpiperidine-4-carboxylate) (5.76 g, 22.3 mmol) and (2,3-dihydro[1,4]dioxino[2,3-c]pyridine-7-carboxaldehyde (for a synthesis see WO2004058144, Example 2(c) or WO2003087098 Example 19(d)) (3.61 g, 21.8 mmol) in DMF (60 mL) at 60° C. was added sodium triacetoxy borohydride (14.2 g, 66.9 mmol) portionwise with cooling as necessary to control effervescence. The mixture was then stirred at RT for 2 h then heated to 67° C. overnight. The mixture was cooled then partitioned between 1:1 water/saturated brine (100 mL) and ethyl acetate (100 mL). The aqueous layer was made basic by addition of saturated sodium bicarbonate solution then extracted with ethyl acetate (3×100 mL). The combined organics were separated and washed with water (100 mL), then brine (100 mL), dried over magnesium sulphate, filtered and evaporated to dryness. Chromatography on silica gel eluting with a gradient of 80-100% ethyl acetate/hexane gave the title compound as a yellow oil (6.82 g).

MS (ES+), m/z 408 (MH⁺, 100%).

### (b) Methyl 4-[(2,3-dihydro[1,4]dioxino[2,3-c]pyridin-7-ylmethyl)amino]-4-piperidinecarboxylate

1-(1,1-Dimethylethyl) 4-methyl 4-[(2,3-dihydro[1,4]dioxino[2,3-c]pyridin-7-ylmethyl)amino]-1,4-piperidinedicarboxylate (0.5 g, 1.2 mmol) was treated with DCM (10 mL) and trifluoroacetic acid (2 mL) and stirred at RT for 18 h. The mixture was evaporated to dryness and the residue dissolved in methanol (50 mL) and treated with amberlyst A-21 ion exchange resin (approx 5 g) and stirred at RT for 30 mins. The resin was removed by filtration and the filtrate was evaporated to dryness. The residue obtained was partitioned between saturated potassium carbonate in water (10 mL) and ethyl acetate (3×30 mL). The combined organic extracts were dried over sodium sulphate filtered and evaporated to dryness to give the title compound as a colourless oil (0.32 g).

MS (ES+), m/z 308 (MH⁺, 80%).

### (c) Methyl 4-[(2,3-dihydro[1,4]dioxino[2,3-c]pyridin-7-ylmethyl)amino]-1-{[(6R)-3-fluoro-8-oxo-5,6-dihydro-8H-[1,4]oxazino[2,3,4-de]-1,5-naphthyridin-6-yl]methyl}-4-piperidinecarboxylate

[(6S)-3-Fluoro-8-oxo-5,6-dihydro-8H-[1,4]oxazino[2,3,4-de]-1,5-naphthyridin-6-yl]methyl methanesulfonate (for a preparation see Example 8(g)) (0.22 g, 0.7 mmol) in acetonitrile (20 mL) was treated with methyl 4-[(2,3-dihydro[1,4]dioxino[2,3-c]pyridin-7-ylmethyl)amino]-4-piperidinecarboxylate (0.32 g, 1.0 mmol) and pyridine (0.1 mL) at 75° C. for 20 h. The mixture was evaporated to dryness and the residue partitioned between saturated sodium bicarbonate solution (50 mL) and DCM (2×50 mL). The combined organics were dried over sodium sulphate filtered and evaporated to dryness. Chromatography on silica gel eluting with a gradient of 0-10% methanol in DCM gave an impure product which was re-chromatographed on silica gel using a gradient of 0-6% methanol in DCM to give the title compound initially as an oil then a white foam (0.14 g).

MS (ES+), m/z 526 (MH⁺, 100%).

### (d) Title Compound

Methyl 4-[(2,3-dihydro[1,4]dioxino[2,3-c]pyridin-7-ylmethyl)amino]-1-{[(6R)-3-fluoro-8-oxo-5,6-dihydro-8H-[1,4]oxazino[2,3,4-de]-1,5-naphthyridin-6-yl]methyl}-4-piperidinecarboxylate (0.17 g, 0.32 mmol) was dissolved in methanol (15 mL) and treated with sodium borohydride (0.25 g, 6.5 mmol) portionwise then stirred at RT for 18 h. More sodium borohydride (0.5 g) was added and the mixture heated at 50° C. for 4 h then a further portion of sodium borohydride (0.25 g) was added and the mixture stirred at RT for 60 h. 5M Hydrochloric acid (10 mL) was added and the mixture stirred at RT for 1 h. The methanol was removed in-vacuo and the mixture basified with solid potassium carbonate to saturation then extracted with 10% methanol/DCM (3×40 mL). The combined organic extracts were dried over sodium sulphate, filtered and evaporated to dryness. Chromatography on silica gel eluting with a gradient 2-14% (2M NH3/MeOH/) DCM gave the free base of the title compound as a colourless oil (0.084 g).

MS (ES+), m/z 498 (MH⁺, 70%).

The free base of the title compound was dissolved in methanol (5 mL) and treated with excess 4M hydrogen chloride in 1,4-dioxane then evaporated to dryness to give the title compound as a white solid.

### Example 82

### N-(1-{[(6R)-3-Fluoro-8-oxo-5,6-dihydro-8H-[1,4]oxazino[2,3,4-de]-1,5-naphthyridin-6-yl]methyl}-4-piperidinyl)-3-oxo-3,4-dihydro-2H-pyrido[3,2-b][1,4]oxazine-6-carboxamide hydrochloride

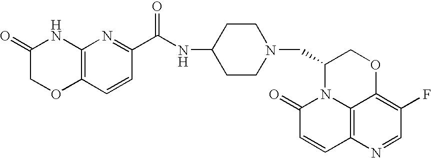

### (a) (6R)-6-[(4-Amino-1-piperidinyl)methyl]-3-fluoro-5,6-dihydro-8H-[1,4]oxazino[2,3,4-de]-1,5-naphthyridin-8-one trifluoroacetate salt

1,1-Dimethylethyl (1-{[(6R)-3-fluoro-8-oxo-5,6-dihydro-8H-[1,4]oxazino[2,3,4-de]-1,5-naphthyridin-6-yl]methyl}-4-piperidinyl)carbamate for a preparation see Example 8(h)) (0.47 g, 1.1 mmole) in DCM (20 mL) was treated with trifluoroacetic acid (5 mL) and stirred at RT for 18 h. The mixture was evaporated to dryness and the residue dissolved in methanol (50 mL) and treated with Amberlyst A-21 ion exchange resin (approx 15 g) and stirred at RT for 1 h. The resin was removed by filtration and the filtrate was evaporated to dryness to give the title compound as a white foam (0.468 g).

MS (ES+), m/z 319 (MH⁺, 100%).

### (b) Title Compound

(6R)-6-[(4-Amino-1-piperidinyl)methyl]-3-fluoro-5,6-dihydro-8H-[1,4]oxazino[2,3,4-de]-1,5-naphthyridin-8-one trifluoroacetate salt (0.07 g, 0.16 mmole) and 3-oxo-3,4-dihydro-2H-pyrido[3,2-b][1,4]oxazine-6-carboxylic acid (for a synthesis see WO2004058144 Example 65) (0.031 g, 0.16 mmole) in DMF (2 mL) were treated with triethylamine (0.05 mL, 0.36 mmole) and O-(7-azabenzotriazol-1-yl)-N,N,N′,N′-tetramethyluroniumhexafluorophosphate (HATU) (0.068 g, 0.18 mmole) and stirred at RT for 18 h. The mixture was evaporated to dryness and the residue partitioned between saturated sodium bicarbonate solution (20 mL) and DCM (2×20 mL). The organics were separated and dried over sodium sulphate. Chromatography on silica-gel eluting with a gradient of 0-10% methanol/DCM gave the free base of the title compound as a white solid (0.064 g).

MS (ES+), m/z 495 (MH⁺, 100%).

The free base of the title compound was dissolved in methanol (5 mL) and treated with excess 4M hydrogen chloride in 1,4-dioxane then evaporated to dryness to give the title compound as a white solid.

### Example 83

### N-(1-{[(6R)-3-fluoro-8-oxo-5,6-dihydro-8H-[1,4]oxazino[2,3,4-de]-1,5-naphthyridin-6-yl]methyl}-4-piperidinyl)-3-oxo-3,4-dihydro-2H-pyrido[3,2-b][1,4]thiazine-6-carboxamide hydrochloride salt

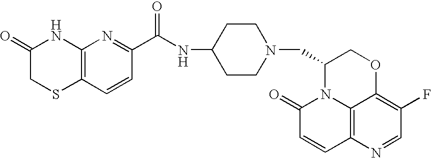

The title compound was prepared as in the general method of Example 82(b) from (6R)-6-[(4-Amino-1-piperidinyl)methyl]-3-fluoro-5,6-dihydro-8H-[1,4]oxazino[2,3,4-de]-1,5-naphthyridin-8-one trifluoroacetate salt (for a preparation see Example 82(a)) (0.07 g, 0.16 mmole) and 3-oxo-3,4-dihydro-2H-pyrido[3,2-b][1,4]thiazine-6-carboxylic acid (for a synthesis see WO2004058144 Example 7(b)) (0.034 g, 0.16 mmole) to give the free base as a white solid (0.074 g).

MS (ES+) m/z 511 (MH⁺, 100%).

The free base of the title compound was dissolved in methanol (5 mL) and treated with excess 4M hydrogen chloride in 1,4-dioxane then evaporated to dryness to give the title compound as a white solid.

### Example 84

### (6R)-6-[((3S)-3-{[(2,3-Dihydro[1,4]dioxino[2,3-c]pyridin-7-ylmethyl)amino]methyl}-1-pyrrolidinyl)methyl]-3-fluoro-5,6-dihydro-8H-[1,4]oxazino[2,3,4-de]-1,5-naphthyridin-8-one dihydrochloride salt

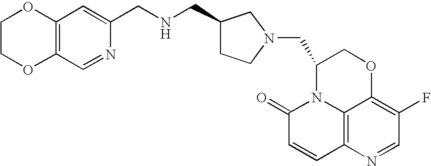

### (a) 1,1-Dimethylethyl [((3S)-1-{[(6R)-3-fluoro-8-oxo-5,6-dihydro-8H-[1,4]oxazino[2,3,4-de]-1,5-naphthyridin-6-yl]methyl}-3-pyrrolidinyl)methyl]carbamate

[(6S)-3-Fluoro-8-oxo-5,6-dihydro-8H-[1,4]oxazino[2,3,4-de]-1,5-naphthyridin-6-yl]methyl methanesulfonate (for a preparation see Example 8(g)) (0.2 g, 0.63 mmol) was converted to the title compound on treatment with 1,1-dimethylethyl [(3R)-3-pyrrolidinylmethyl]carbamate (0.25 g, 1.2 mmol) (prepared from its hydrochloride salt by neutralisation with potassium carbonate and extraction into 10% methanol/DCM)) under the general conditions described for Example 7(e). After work-up and chromatography the desired product was obtained as a yellow oil (0.037 g).

MS (ES+), m/z 419 (MH⁺, 60%).

### (b) Title Compound

1,1-Dimethylethyl [((3S)-1-{[(6R)-3-fluoro-8-oxo-5,6-dihydro-8H-[1,4]oxazino[2,3,4-de]-1,5-naphthyridin-6-yl]methyl}-3-pyrrolidinyl)methyl]carbamate (0.037 g, 0.09 mmol) was dissolved in DCM (4 mL) and treated with trifluoroacetic acid (1 mL) and stirred at RT for 18 h. The mixture was evaporated to dryness and the residue partitioned between saturated potassium carbonate solution (10 mL) and 10% methanol/DCM (3×15 mL). The organics were dried over sodium sulphate, filtered then evaporated to dryness to give a mixture containing the deprotected compound as a brown oil. This was treated with (2,3-dihydro[1,4]dioxino[2,3-c]pyridine-7-carboxaldehyde (for a synthesis see WO2004058144, Example 2(c) or WO2003087098 Example 19(d)) (0.008 g, 0.05 mmol) under the general conditions of Example 8(j). The free base of the title compound was obtained as a colourless oil (0.014 g).

MS (ES+) m/z 468 (MH⁺, 100%).

The free base of the title compound was dissolved in methanol (2 mL) and treated with excess 4M hydrogen chloride in 1,4-dioxane then evaporated to dryness to give the title compound as a yellow solid.

### Example 85

### (6R)-6-[((3S,4R)-3-{[(3,4-dihydro-2H-pyrano[2,3-c]pyridin-6-ylmethyl)amino]methyl}-4-hydroxy-1-piperidinyl)methyl]-3-fluoro-5,6-dihydro-8H-[1,4]oxazino[2,3,4-de]-1,5-naphthyridin-8-one dihydrochloride salt

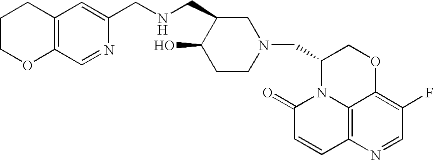

(6R)-6-{[3-(Aminomethyl)-4-hydroxy-1-piperidinyl]methyl}-3-fluoro-5,6-dihydro-8H-[1,4]oxazino[2,3,4-de]-1,5-naphthyridin-8-one (for a preparation see Example 26(j)) (0.093 g, 0.26 mmol) was treated with 3,4-dihydro-2H-pyrano[2,3-c]pyridine-6-carbaldehyde (for a synthesis see WO2004058144 Example 126(e)) (0.039 g, 0.24 mmol) under the general conditions of Example 8(j) omitting sodium acetate. The free base of the title compound was obtained as a colourless oil (0.036 g).

MS (ES+) m/z 496 (MH⁺, 100%).

The free base of the title compound was dissolved in methanol (3 mL) and treated with excess 4M hydrogen chloride in 1,4-dioxane then evaporated to dryness to give the title compound as a white solid.

### Example 86

### (6R)-6-[((3R)-3-{[(2,3-dihydro[1,4]dioxino[2,3-c]pyridin-7-ylmethyl)amino]methyl}-1-pyrrolidinyl)methyl]-3-fluoro-5,6-dihydro-8H-[1,4]oxazino[2,3,4-de]-1,5-naphthyridin-8-one dihydrochloride salt

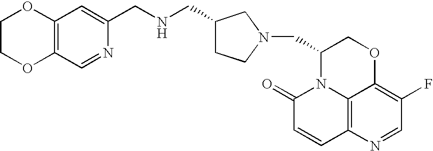

### (a) 1,1-Dimethylethyl [((3R)-1-{[(6R)-3-fluoro-8-oxo-5,6-dihydro-8H-[1,4]oxazino[2,3,4-de]-1,5-naphthyridin-6-yl]methyl}-3-pyrrolidinyl)methyl]carbamate

[(6S)-3-Fluoro-8-oxo-5,6-dihydro-8H-[1,4]oxazino[2,3,4-de]-1,5-naphthyridin-6-yl]methyl methanesulfonate (for a preparation see Example 8(g)) (0.365 g, 1.1 mmol) was converted to the title compound on treatment with 1,1-dimethylethyl [(3S)-3-pyrrolidinylmethyl]carbamate (0.465 g, 2.3 mmol) (prepared from its hydrochloride salt by neutralisation with potassium carbonate and extraction into 10% methanol/DCM)) under the standard conditions described for Example 7(e). After work-up and chromatography the desired product was obtained as a colourless oil (0.117 g).

MS (ES+), m/z 419 (MH⁺, 100%).

### (b) Title Compound

1,1-Dimethylethyl [((3R)-1-{[(6R)-3-fluoro-8-oxo-5,6-dihydro-8H-[1,4]oxazino[2,3,4-de]-1,5-naphthyridin-6-yl]methyl}-3-pyrrolidinyl)methyl]carbamate (0.117 g, 0.28 mmol) was dissolved in DCM (10 mL) and treated with trifluoroacetic acid (2 mL) and stirred at RT for 4 h. The mixture was evaporated to dryness and the residue dissolved in methanol (30 mL) and treated with Amberlyst A-21 ion exchange resin (approx 10 g) and stirred at RT for 2 h. The resin was removed by filtration and the filtrate was evaporated to dryness to give (6R)-6-{[(3R)-3-(aminomethyl)-1-pyrrolidinyl]methyl}-3-fluoro-5,6-dihydro-8H-[1,4]oxazino[2,3,4-de]-1,5-naphthyridin-8-one mono trifluoroacetate salt as a light brown oil (0.107 g). Half of this material (0.05 g, 0.11 mmol) was treated with (2,3-dihydro[1,4]dioxino[2,3-c]pyridine-7-carboxaldehyde (for a synthesis see WO2004058144, Example 2(c) or WO2003087098 Example 19(d)) (0.019 g, 0.11 mmol) under the general conditions of Example 8(j). The free base of the title compound was obtained as a colourless oil (0.043 g).

MS (ES+) m/z 468 (MH⁺, 100%).

The free base of the title compound was dissolved in methanol (2 mL) and treated with excess 4M hydrogen chloride in 1,4-dioxane then evaporated to dryness to give the title compound as a yellow solid.

### Example 87

### (6R)-6-[((3R)-3-{[(6,7-dihydro[1,4]dioxino[2,3-c]pyridazin-3-ylmethyl)amino]methyl}-1-pyrrolidinyl)methyl]-3-fluoro-5,6-dihydro-8H-[1,4]oxazino[2,3,4-de]-1,5-naphthyridin-8-one dihydrochloride salt

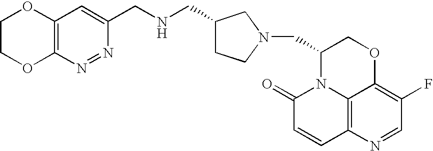

(6R)-6-{[3R)-3-(aminomethyl)-1-pyrrolidinyl]methyl}-3-fluoro-5,6-dihydro-8H-[1,4]oxazino[2,3,4-de]-1,5-naphthyridin-8-one trifluoroacetate salt (for a preparation see Example 86 (b) (0.05 g, 0.11 mmol) was treated with 6,7-dihydro[1,4]dioxino[2,3-c]pyridazine-3-carbaldehyde ((for a preparation see Example 3(e) (0.019 g, 0.11 mmol) under the general conditions of Example 8(j). The free base of the title compound was obtained as a colourless oil (0.041 g).

MS (ES+) m/z 469 (MH⁺, 100%).

The free base of the title compound was dissolved in methanol (2 mL) and treated with excess 4M hydrogen chloride in 1,4-dioxane then evaporated to dryness to give the title compound as a yellow solid.

### Example 91

### 6-({4-[(2,3-dihydro[1,4]dioxino[2,3-c]pyridin-7-ylmethyl)amino]-1-piperidinyl}methyl)-3-(methyloxy)-5,6-dihydro-4H,8H-pyrido[3,2,1-de]-1,5-naphthyridin-8-one hydrochloride

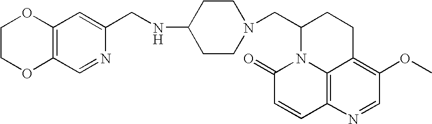

6-({4-[(2,3-Dihydro[1,4]dioxino[2,3-c]pyridin-7-ylmethyl)amino]-1-piperidinyl}methyl)-3-fluoro-5,6-dihydro-4H,8H-pyrido[3,2,1-de]-1,5-naphthyridin-8-one (for a synthesis see example 15) (30 mg, 0.06 mmol) was dissolved in methanol (14 ml) and treated with a solution of sodium methoxide in methanol (0.14 ml of 25% w/v solution) and stirred at room temperature overnight. The solvent was evaporated and the residue chromatographed silica eluting with 0-10% 2M methanolic ammonia in DCM to afford the free base of the title compound (24 mg, 83%).

MS (ES+), m/z 478 (MH⁺, 20%), 500 (MNa+, 10%)

The free base was dissolved in methanol and treated with 1M HCl in ether (0.05 mmol, 1.0 equiv). The excess solvent was removed under reduced pressure and the residue triturated to afford the title compound as a solid.

### Example 92

### 3-(Methyloxy)-6-({4-[([1,3]oxathiolo[5,4-c]pyridin-6-ylmethyl)amino]-1-piperidinyl}methyl)-5,6-dihydro-4H,8H-pyrido[3,2,1-de]-1,5-naphthyridin-8-one hydrochloride

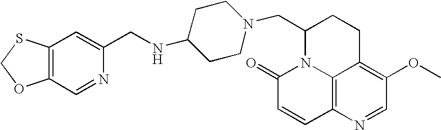

3-Fluoro-6-({4-[([1,3]oxathiolo[5,4-c]pyridin-6-ylmethyl)amino]-1-piperidinyl}methyl)-5,6-dihydro-4H,8H-pyrido[3,2,1-de]-1,5-naphthyridin-8-one (free base of Example 60, Table 7) was reacted with sodium methoxide under the general conditions of Example 91 to give the free base of the title compound (25 mg, 86%).

MS (ES+), m/z 480 (MH⁺, 100%).

The free base was dissolved in methanol and treated with 1M HCl in ether (0.05 mmol, 1.0 equiv). The excess solvent was removed under reduced pressure to afford the title compound as a solid.

### Example 93

### 6-({4-[(2,3-dihydro[1,4]dioxino[2,3-c]pyridin-7-ylmethyl)amino]-1-piperidinyl}methyl)-3-fluoro-5,6-dihydro-4H,8H-pyrido[3,2,1-de]-1,5-naphthyridin-8-one Enantiomer 2-benzoate

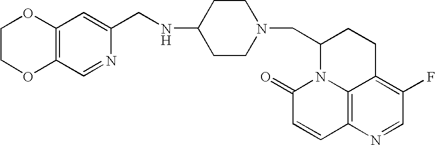

6-({4-[(2,3-dihydro[1,4]dioxino[2,3-c]pyridin-7-ylmethyl)amino]-1-piperidinyl}methyl)-3-fluoro-5,6-dihydro-4H,8H-pyrido[3,2,1-de]-1,5-naphthyridin-8-one Enantiomer 2 (positive peak on polarimeter) (for a preparation see Example 17) was converted to the benzoate salt to give the title compound as a white solid.

### Example 94

### 6-({4-[(2,3-dihydro[1,4]dioxino[2,3-c]pyridin-7-ylmethyl)amino]-1-piperidinyl}methyl)-3-fluoro-5,6-dihydro-4H,8H-pyrido[3,2,1-de]-1,5-naphthyridin-8-one Enantiomer 2 dihydrochloride

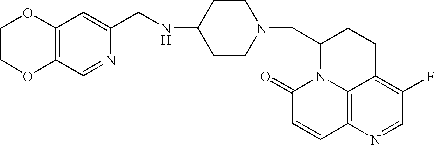

6-({4-[(2,3-dihydro[1,4]dioxino[2,3-c]pyridin-7-ylmethyl)amino]-1-piperidinyl}methyl)-3-fluoro-5,6-dihydro-4H,8H-pyrido[3,2,1-de]-1,5-naphthyridin-8-one Enantiomer 2 (positive peak on polarimeter) (36 mg, 0.077 mmol) (for a preparation see Example 17) was dissolved in methanol (2 ml) and treated with 1M HCl in ether (0.2 ml, 3 equiv). The solvent was removed under reduced pressure to give the di-hydrochloride as a pale yellow solid.

MS (ES+), m/z 466 (MH+, 100%)

### Example 95

### 6-({4-[(2,3-dihydro[1,4]dioxino[2,3-c]pyridin-7-ylmethyl)amino]-1-piperidinyl}methyl)-3-fluoro-5,6-dihydro-4H,8H-pyrido[3,2,1-de]-1,5-naphthyridin-8-one Enantiomer 1 dihydrochloride

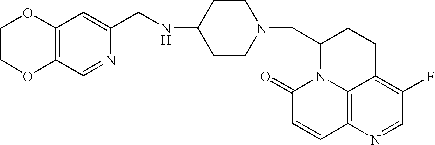

6-({4-[(2,3-dihydro[1,4]dioxino[2,3-c]pyridin-7-ylmethyl)amino]-1-piperidinyl}methyl)-3-fluoro-5,6-dihydro-4H,8H-pyrido[3,2,1-de]-1,5-naphthyridin-8-one Enantiomer 1 (negative peak on polarimeter) (for a preparation see Example 16) (32 mg, 0.068 mmol) was dissolved in methanol (2 ml) and treated with 1M HCl in ether (0.2 ml, 3 equiv). The solvent was removed under reduced pressure to give the di-hydrochloride as a pale yellow solid.

MS (ES+), m/z 466 (MH+, 100%)

### Example 96

### (6R)-6-[((3S,4S)-3-{[(2,3-dihydro[1,4]dioxino[2,3-c]pyridin-7-ylmethyl)amino]methyl}-4-hydroxy-1-pyrrolidinyl)methyl]-3-fluoro-5,6-dihydro-4H,8H-pyrido[3,2,1-de]-1,5-naphthyridin-8-one dihydrochloride (single diastereomer)

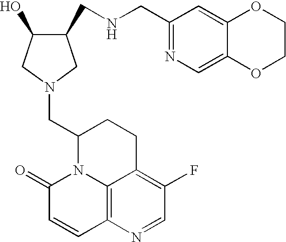

### (a) 3-Fluoro-6-(hydroxymethyl)-5,6-dihydro-4H,8H-pyrido[3,2,1-de]-1,5-naphthyridin-8-one Enantiomer 1 and Enantiomer 2

Racemic 3-fluoro-6-(hydroxymethyl)-5,6-dihydro-4H,8H-pyrido[3,2,1-de]-1,5-naphthyridin-8-one (for a preparation see Example 15(f)) (4.43 g) was separated into its enantiomers by chiral preparative HPLC on a 4-inch chiralpak AD (20 micron) column eluting with 0.1% isopropylamine in methanol. The faster-running enantiomer (E1) was obtained as a white solid (1.92 g)

MS (ES+), m/z 235 (MH+, 100%). optical rotation α_{D} −173.2 degrees (22° C., C 1.00, methanol), chiral purity >99%.

The slower running enantiomer was obtained as a white solid (1.95 g).

MS (ES+), m/z 235 (MH+, 100%). Optical rotation α_{D} +174.6 degrees (22° C., C 1.00, methanol), chiral purity 99%.

### (b) (3-Fluoro-8-oxo-5,6-dihydro-4H,8H-pyrido[3,2,1-de]-1,5-naphthyridin-6-yl)methyl methanesulfonate (derived from hydroxymethyl Enantiomer 2)

3-Fluoro-6-(hydroxymethyl)-5,6-dihydro-4H,8H-pyrido[3,2,1-de]-1,5-naphthyridin-8-one Enantiomer 2 (234 mg, 1.0 mmol) was converted to the methanesulfonate under the general conditions described for Example 19 (b) to give the title compound (330 mg, >100%)

MS (ES+), m/z 313 (MH+, 100%)

### (c) 1,1-Dimethylethyl ({(3S,4S)-1-[(3-fluoro-8-oxo-5,6-dihydro-4H,8H-pyrido[3,2,1-de]-1,5-naphthyridin-6-yl)methyl]-4-hydroxy-3-pyrrolidinyl}methyl)carbamate

(3-Fluoro-8-oxo-5,6-dihydro-4H,8H-pyrido[3,2,1-de]-1,5-naphthyridin-6-yl)methyl methanesulfonate (derived from hydroxymethyl Enantiomer 2) (0.326 g, containing 1.0 mmol) was reacted with phenylmethyl {[(3R,4S)-4-hydroxy-3-pyrrolidinyl]methyl}carbamate (for a synthesis see WO2006002047, preparation 24(d) Enantiomer 1) (0.39 g, 1.56 mmol) under the general conditions described for Example 7(e). After work-up and chromatography the title compound was obtained as a pale brown oil (0.290 g, 62%).

### (d) 6-{[(3S,4S)-3-(Aminomethyl)-4-hydroxy-1-pyrrolidinyl]methyl}-3-fluoro-5,6-dihydro-4H,8H-pyrido[3,2,1-de]-1,5-naphthyridin-8-one

1,1-Dimethylethyl ({(3S,4S)-1-[(3-fluoro-8-oxo-5,6-dihydro-4H,8H-pyrido[3,2,1-de]-1,5-naphthyridin-6-yl)methyl]-4-hydroxy-3-pyrrolidinyl}methyl)carbamate (290 mg, 0.62 mmol) was hydrogenated under the general conditions described for Example 28(c) except that the reaction time was increased to 18 h. The crude product was chromatographed on silica eluting with 0-20% 2M methanolic ammonia in DCM to afford the title compound as a white solid (96 mg, 46%).

MS (ES+), m/z 335 (MH⁺, 100%).

### (e) Title Compound

6-{[(3S,4S)-3-(Aminomethyl)-4-hydroxy-1-pyrrolidinyl]methyl}-3-fluoro-5,6-dihydro-4H,8H-pyrido[3,2,1-de]-1,5-naphthyridin-8-one (0.046 g, 0.14 mmol) was converted to the free base of the title compound on treatment with 2,3-dihydro[1,4]dioxino[2,3-c]pyridine-7-carbaldehyde (for a synthesis see WO2004058144, Example 2(c) or WO2003087098 Example 19(d) (0.021 g, 0.13 mmole) under the general conditions described for Example 1(m) to afford the free base of the title compound (16 mg, 23%)

MS (ES+), m/z 484 (MH⁺, 50%).

The free base of the title compound (16 mg) was converted into the dihydrochloride salt by dissolving in methanol (2 ml) and treatment with 1M HCl in ether (0.1 ml, 3 equiv). The solvent was evaporated and the residue triturated with diethyl ether (3 ml) to afford the title compound as a solid.

The following compounds of Table 1-8 were prepared in a similar manner to previous Examples as follows:

### Table 1 Compounds 31-36, 90 Example 12;

### Table 2 Compounds 37-41, 97 Example 20;

### Table 3 Compounds 42-44 Example 21;

### Table 4 Compounds 45-49, 88 Example 8;

### Table 5 Compounds 50-54 Example 22;

### Table 6 Compounds 55-58, 89 Example 23;

### Table 7 Compounds 59-66 Example 15;

### Table 8 Compounds 67-68 Example 30;

from the specified aldehyde.

TABLE 1

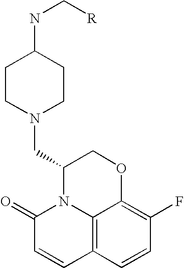

| Exam- | | | | MS ES+ | Nmr of free base |
|---|---|---|---|---|---|
| ple | R | Name | Aldehyde | m/z | (δ (ppm), CDCl₃, 400 MHz) |
| 31 | 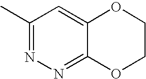 | (3R)-3-({4-[(6,7- dihydro[1,4]dioxino[2,3- c]pyridazin-3-ylmethyl)amino]- 1-piperidinyl}methyl)-10- fluoro-2,3-dihydro-5H- [1,4]oxazino[2,3,4-ij]quinolin- 5-one | Example 3(e) | 468 MH+, 100% | 1.64-1.86 (4H, s), 1.89-1.93 (1H, m), 2.19-2.28 (1H, m), 2.31-2.40 (1H, m), 2.42-2.51 (1H, m), 2.53-2.62 (2H, m), 2.84-2.87 (1H, m), 3.17-3.21 (1H, m), 4.00 (3H, s), 4.36-4.40 (2H, m), 4.51- 4.55 (2H, m), 4.90-5.0 (2H, m), 6.61 (1H,d, J = 8 Hz), 6.99-7.05 (2H, m), 7.09- 7.13 (1H, m), 7.64 (1H, d, J = 8 Hz) |
| 32 | 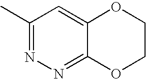 | (3R)-3-({4-[(6,7- dihydro[1,4]dioxino[2,3- c]pyridazin-3-ylmethyl)amino]- 1-piperidinyl}methyl)-10- fluoro-2,3-dihydro-5H- [1,4]oxazino[2,3,4-ij]quinolin- 5-one benzoate salt | Example 3(e) | | |
| 33 | 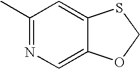 | (3R)-1-fluoro-3-({4- [([1,3]oxathiolo[5,4-c]pyridin-6- ylmethyl)amino]-1- piperidinyl}methyl)-2,3- dihydro-5H-[1,4]oxazino[2,3,4- ij]quinolin-5-one hydrochloride | WO20044058144 Example 61 | 469 MH+, 30%. | δ (ppm 250 MHz) 1.41-1.52 (2H, m), 1.85-1.90 (2H, m), 2.10-2.20 (2H, m), 2.29-2.37 (4H, m), 2.52-2.63 (1H, m), 2.84-2.89 (1H, m), 3.18-3.23 (1H, br, d), 3.44-3.52 (1H, m), 3.84 (2H, s), 3.99- 4.03 (1H, m), 4.92-5.00(1H, br, d), 5.72 (2H, s), 6.61 (1H, d, J = 10 Hz), 6.95-6.99 (1H, m), 7.03-7.12 (1H, m), 7.64 (1H, d, J = 10 Hz), 8.00 (1H, s). |
| 34 | 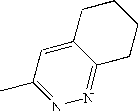 | (3R)-10-fluoro-3-({4-[(5,6,7,8- tetrahydro-3- cinnolinylmethyl)amino]-1- piperidinyl}methyl)-2,3- dihydro-5H-[1,4]oxazino[2,3,4- ij]quinolin-5-one fumarate salt | Intermediate 1 | 464 MH+, 20%. | 1.35-1.50 (2H, m), 1.78-1.96 (6H, m), 2.17 (1H, dt, J = 2.4, 11.6 Hz), 2.29 (1H, dt, J = 2.4, 11.2 Hz), 2.41-2.42 (1H, m), 2.51-2.60 (2H, m), 2.72 (2H, t, J = 6.4 Hz), 2.85-2.90 (1H, m), 3.11 (2H, t, J = 6.4 Hz), 3.17-3.22(1H m), 3.96- 3.98 (1H, m), 4.03 (2H, s), 4.92-5.97 (2H, m), 6.61 (1H, d, J = 9.6 Hz), 6.96- 7.00 (1H, m), 7.09- .10 (1H, m), 7.18 (1H, s), 7.63 (1H, d, J = 9.6 Hz) |
| 35 | 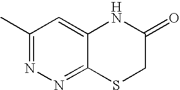 | (3R)-10-fluoro-3-[(4-{[(6-oxo- 6,7-dihydro-5H-pyridazino[3,4- b][1,4]thiazin-3- yl)methyl]amino}-1- piperidinyl)methyl]-2,3- dihydro-5H-[1,4]oxazino[2,3,4- ij]quinolin-5-one fumarate salt | WO2004058144 Example 58(d) | 497 MH+, 100%. | 1.50-1.65 (2H, m), 1.99-2.15 (2H, m), 2.20-2.280?? (1H, m), 2.33-2.40 (1H, m), 2.43-2.44 (1H, m), 2.60-2.63 (1H, m), 2.78-2.90 (2H, m), 2.93-3.08 (1H, m), 3.12-3.22 (1H, m), 3.63 (2H,s), 4.02-4.12 (1H, m), 4.25 (2H, s), 4.46- 4.96 (2H, m), 6.65 (1H, d, J = 8 Hz), 6.98- 7.02 (1H, m), 7.11-7.15 (1H, m), 7.25 (1H, m), 7.67 (1H, d, J = 8 Hz). |
| 36 | 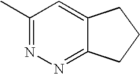 | (3R)-3-({4-[(6,7-dihydro-5H- cyclopenta[c]pyridazin-3- ylmethyl)amino]-1- piperidinyl}methyl)-10-fluoro- 2,3-dihydro-5H- [1,4]oxazino[2,3,4-ij]quinolin- 5-one hydrochloride | Intermediate 2 | 450 MH+, 20%. | 1.92-1.95 (2H, m), 2.10-2.25 (6H, m), 2.35-2.42 (2H, m), 2.52-2.60 (2H, m), 3.12-3.2 (4H, m), 3.49-4.400?? (1H, m), 4.07 (1H, s), 4.91-4.99 (2H, m), 6.62 (1H, d, J = 12 Hz), 6.97-6.99 (1H, m), 7.37 (1H, s), 7.64(1H, d, J = 12 Hz). |
| 90. | 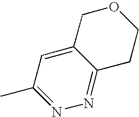 | (6R)-6-({4-[(7,8-dihydro-5H- pyrano[4,3-c]pyridazin-3- ylmethyl)amino]-1- piperidinyl}methyl)-3-fluoro- 5,6-dihydro-8H- [1,4]oxazino[2,3,4-de]-1,5- naphthyridin-8-one fumaric acid salt | Intermediate 3 | | |

TABLE 2

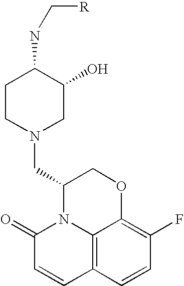

| Exam- | | | | MS ES+ | Nmr of free base |
|---|---|---|---|---|---|
| ple | R | Name | Aldehyde | m/z | (δ (ppm), CDCl3, 400 MHz) |
| 37 | 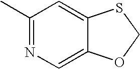 | (3R)-10-fluoro-3-({(3R,4S)-3- hydroxy-4- [([1,3]oxathiolo[5,4-c]pyridin- 6-ylmethyl)amino]-1- piperidinyl}methyl)-2,3- dihydro-5H- [1,4]oxazino[2,3,4-ij]quinolin- 5-one hydrochloride | WO2004058144 Example 61 | 485 MH+, 10%. | 1.69-1.72 (5H, m), 2.31-2.36 (2H, m), 2.46-2.50 (1H, m), 2.65-2.71 (1H, m), 2.84-2.95 (1H, m), 3.21-3.25 (1H, m), 3.86 (3H, s), 4.03-4.06 (1H, m), 4.91- 4.92 (1H, m), 4.97-5.01 (1H, m), 5.74 (2H,s), 6.61 (1H, d, J = 12 Hz), 6.97-7.02 (2H, m), 7.11-7.22 (1H, m), 7.26 (1H, s), 7.64 (1H, d, J = 12 Hz), 8.00 (1H, m). |
| 38 | 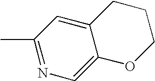 | (3R)-3-({(3R,4S)-4-[(3,4- dihydro-2H-pyrano[2,3- c]pyridin-6-ylmethyl)amino]- 3-hydroxy-1- piperidinyl}methyl)-10-fluoro- 2,3-dihydro-5H- [1,4]oxazino[2,3,4-ij]quinolin- 5-one hydrochloride | WO2004058144 Example 126(e) | 481 MH+, 10% | 1.71-1.80 (4H, m), 2.02-2.10 (2H, m), 2.31-2.36 (2H, m), 2.45-2.51 (1H, m), 2.56-2.58 (1H, m), 2.60-2.68 (1H, m), 2.70-2.79 (2H, m), 2.81-2.91 (1H, m), 3.23-3.26 (1H, m), 3.49 (2H, s), 3.83- 3.86 (2H, m), 4.03-4.06 (1H, m), 4.10- 4.22 (2H, m), 4.9-5.00 (2H, m), 6.61 (1H, d, J = 12 Hz), 6.97-6.99 (2H, m), 7.02-10?? (1H, m), 7.64 (1H, d, J = 12 Hz), 8.07 (1H, s). |
| 39 | 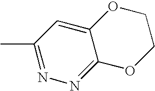 | (3R)-3-({(3R,4S)-4-[(6,7- dihydro[1,4]dioxino[2,3- c]pyridazin-3- ylmethyl)amino]-3-hydroxy-1- piperidinyl}methyl)-10-fluoro- 2,3-dihydro-5H- [1,4]dioxino[2,3,4-ij]quinolin- 5-one fumarate (salt) | Example 3(e) | 484 MH+, 100% | 1.70-1.80 (4H, m), 2.25-2.34 (2H, m), 2.47-2.55 (2H, m), 2.65-2.67 (1H, m), 2.84-2.93 (1H, m), 3.20-3.23 (1H, m), 3.85 (1H, s), 4.00-4.10 (3H, m), 4.36- 4.39 (2H, m), 4.50-5.54 (2H, m), 4.86- 4.90(1H, m), 5.00-5.03 (1H, m), 6.61 (1H, d, J = 12 Hz), 6.97-7.02 (1H, m), 7.08-7.14 (2H, m), 7.65 (1H, d, J = 12 Hz). |
| 40 | 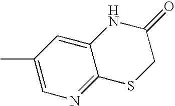 | (3R)-10-fluoro-3-[((3R,4S)-3- hydroxy-4-{[(2-oxo-2,3- dihydro-1H-pyrido[2,3- b][1,4]thiazin-7- yl)methyl]amino}-1- piperidinyl)methyl]-2,3- dihydro-5H- [1,4]oxazino[2,3,4-ij]quinolin- 5-one hydroxychloride | WO2004 058144 Example 48 | 512 MH+, 100% | 1.72-1.85 (3H, br, s), 1.87-1.93 (2H, m), 2.16-2.19 (1H, m), 2.25-2.31 (1H, m), 2.37-2.41 (1H, m), 2.48-2.55 (1H, m), 2.59-2.63 (1H, m), 2.84-2.87 (1H, m), 3.18-3.21(1H, m), 3.78 (2H, s), 4.00- 4.4.03?? (1H, m), 4.28-4.34 (4H, m), 4.95-4.99 (2H, m), 6.63 (1H, d, J = 12 Hz), 6.82 (1H, s), 6.99-7.09 (1H, m), 7.10-7.13 (1H, m), 7.66 (1H, d, J = 12 Hz), 8.10 (1H, s) |
| 41 | 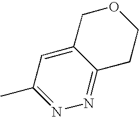 | (3R)-3-({(3R,4S)-4-[(7,8- dihydro-5H-pyrazno[4,3- c]pyridazin-3- ylmethyl)amino]-3-hydroxy-1- piperidinyl}methyl)-10-fluoro- 2,3-dihydro-5H- [1,4]oxazino[2,3,4-ij]quinolin- 5-one fumarate (salt) | Intermediate 3 | 482 MH+, 40% | 1.57-1.78 (2H, m), 2.26-2.35 (2H, m), 2.48 (1H, dd, J = 4.4, 12.4 Hz), 2.51-2.56 (1H, m), 2.65-2.71 (3H, m), 2.89 (1H, d, J = 11.2 Hz), 3.18-3.24 (3H, m), 3.89 (1H, br.s), 4.09-4.13 (3H, m),4.76- 4.78 (2H, m), 4.88 (1H, d, J = 11.6 Hz), 4.96-5.00 (1H, m), 6.61 (1H, d, J = 9.6 Hz), 6.98-7.06 (1H, m), 7.12-7.14 (1H, m), 7.28-7.30 (1H, m), 7.65 (1H, dd, J = 0.8, 9.6 Hz) |

TABLE 3

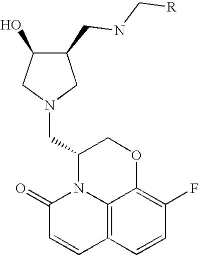

| Exam- | | | | MS ES+ | Nmr of free base |
|---|---|---|---|---|---|
| ple | R | Name | Aldehyde | m/z | (δ (ppm), CDCl3, 400 MHz) |
| 42 | 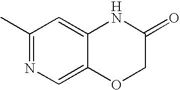 | (3R)-10-fluoro-3-{[(3S,4R)-3- hydroxy-4-({[(2-oxo-2,3- dihydro-1H-pyrido[3,4- b][1,4]oxazin-7- yl)methyl]amino}methyl)-1- pyrrolidinyl]methyl}-2,3- dihydro-5H- [1,4]oxazino[2,3,4-ij]quinolin- 5-one hydrochloride | WO200387098 Example 309(i) | 496 MH+, 100% | 2.39-2.42 (1H, m), 2.55-2.58 (1H, m), 2.74-2.76 (1H, m), 2.80-2.96 (6H, m), 3.49 (3H, s), 3.83-3.92 (2H, m), 4.03- 4.10 (1H, m), 4.42-4.45 (1H, m), 4.66 (2H, m), 4.89-4.92 (1H, m), 5.06-5.10 (1H, m), 6.73 (1H, d, J = 8 Hz), 7.00-7.04 (2H, m), 7.13-7.16 (1H, m), 7.68 (1H, d, J = 8 Hz), 8.18 (1H, s). |
| 43 | 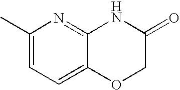 | (3R)-10-fluoro-3-{[(3S,4S)-3- hydroxy-4-({[(3-oxo-3,4- dihydro-2H-pyrido[3,2- b][1,4]oxazin-6- yl)methyl]amino}methyl)-1- pyrrolidinyl]methyl}-2,3- dihydro-5H- [1,4]oxazino[2,3,4-ij]quinolin- 5-one hydrochloride | WO2003087098 Example 31(e) | 496 MH+, 100% | 2.44-2.47 (1H, m), 2.51-2.55 (1H, m), 2.75-2.92 (8H, m), 3.48 (2H, m), 3.76- 3.86 (2H, m), 4.01-4.04 (1H, m), 4.44- 4.48 (1H, m), 4.64 (2H, m), 4.95-4.98 (1H, m), 5.02-5.05 (1H, m), 6.68 (1H, d, J = 8 Hz), 6.87 (1H, d, J = 8 Hz), 6.99-7.04 (1H, m), 7.12-7.14 (1H, m), 7.14 (1H, d, J = 8 Hz), 7.65 (1H, d, J = 8 Hz) |
| 44 | 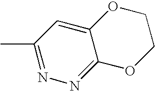 | (3R)-3-[((3S,4S)-3-{[(6,7- dihydro[1,4]dioxino[2,3- c]pyridazin-3- ylmethyl)amino]methyl}-4- hydroxy-1- pyrrolidinyl)methyl]-10- fluoro-2,3-dihydro-5H- [1,4]oxazino[2,3,4-ij]quinolin- 5-one fumarate (salt) | Example 3(e) | 484 MH+, 40%, 506 MNa+, 10%. | 2.40-2.47 (1H, m), 2.50 (1H, m), 2.65- 2.98 (6H, m), 3.97-4.03 (3H, m), 4.36- 4.40 (2H, m), 4.41-4.45 (1H, m), 4.52- 4.55 (2H, m), 4.90 (1H, d, J = 11.2Hz), 5.00-5.04 (1H, m), 6.58 (1H, d, J = 9.6 Hz), 6.95-7.00 (2H, m), 7.10-7.12 (1H, m), 7.62 (1H, d, J = 9.6 Hz) |

TABLE 4

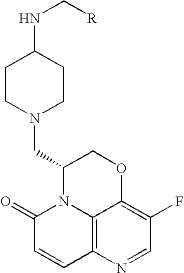

| | | | | MS ES+ | Nmr of free base |
|---|---|---|---|---|---|
| Example | R | Name | Aldehyde | m/z | (δ (ppm), CDCl3, 400 MHz) |
| 45 | 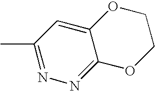 | (6R)-6-({4-[(6,7- dihydro[1,4]dioxino[2,3- c]pyridazin-3- ylmethyl)amino]-1- piperidinyl}methyl)-3-fluoro- 5,6-dihydro-8H- [1,4]oxazino[2,3,4-de]-1,5- naphthyridin-8-one | Example 3(e) | 469 MH+, 100% | 1.51-1.71 (4H, m), 1.87-1.90 (2H, m), 2.23-2.59 (4H, m), 2.81-2.85 (1H, m), 3.12-3.17 (1H, m), 4.00 (2H, s), 4.15- 4.20 (1H, m), 4.35-4.36 (2H, m), 4.51- 4.55 (2H, m), 4.93-4.96 (1H, m), 5.05- 5.10 (1H, m), 6.84 (1H, d,J = 10 Hz), 7.04 (1H, s), 7.86 (1H, d, J = 10 Hz), 8.37 (1H, d, J = 2.5 Hz) |
| 46 | 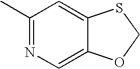 | (6R)-3-fluoro-6-({4- [([1,3]oxathiolo[5,4-c]pyridin- 6-ylmethyl)amino]-1- piperidinyl}methyl)-5,6- dihydro-8H- [1,4]oxazino[2,3,4-de]-1,5- naphthyridin-8-one | WO2004058144 Example 61 | 470 MH+, 100% | 1.42-1.66 (5H, m), 1.86-1.94 (2H, m), 2.14-2.60 (4H, m), 2.81-2.85 (1H, m), 3.14-3.18 (1H, m), 3.82 (2H, s), 4.93- 4.96 (1H, m), 5.05-5.10 (1H, m), 5.73- 5.72 (2H, m), 6.84 (1H, d, J = 10 Hz), 7.12 (1H, s), 7.86 (1H, d, J= 10 Hz), 8.00 (1H, s), 8.37 (1H, d, J = 2.5 Hz). |
| 47 | 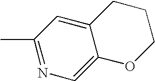 | (6R)-6-({4-[(3,4-dihydro-2H- pyrano[2,3-c]pyridin-6- ylmethyl)amino]-1- piperidinyl}methyl)-3-fluoro- 5,6-dihydro-8H- [1,4]oxazino[2,3,4-de]-1,5- naphthyridin-8-one hydrochloride | WO200458144 Example 126 (e) | 466 MH+, 50% | 1.33-1.58 (2H, m), 1.82-2.62 (9H, m), 2.69-2.89 (3H, m), 3.11-3.21 (1H, m), 3.81 (2H, s), 4.07-4.27 (3H, m), 4.88- 4.98 (1H, m), 5.07 (1H, d, J = 11 Hz), 6.82 (1H, d, J = 10 Hz), 6.98 (1H, s),7.85 (1H, d, J = 10 Hz), 8.07 (1H, s), 8.37 (1H, d, J = 2 Hz). |
| 48 | 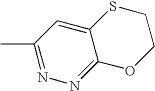 | (6R)-6-({4-[(6,7- dihydro[1,4]oxathiino[2,3- c]pyridazin-3- ylmethyl)amino]-1- piperidinyl}methyl)-3-fluoro- 5,6-dihydro-8H- [1,4]oxazino[2,3,4-de]-1,5- naphthyridin-8-one benzoate | Example 4(d) | 485 MH+, 35% | 1.29-1.52 (2H, m), 1.81-1.97 (2H, m), 2.11-2.61 (5H, m), 2.76-2.87 (1H, m), 3.07-3.25 (3H, m), 3.95 (2H, s), 4.08- 4.17 (1H, m), 4.61-4.68 (2H, m), 4.88- 4.98 (1H, m), 5.06 (1H, d, J = 11 Hz), 6.81 (1H, d, J =10 Hz), 7.3?? (1H, s), 7.83 (1H, d, J = 10 hz), 8.38 (1H, d, J = 2 Hz). |
| 49 | 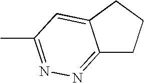 | (6R)-6-({4-[(6,7-dihydro-5H- cyclopenta[c]pyridazin-3- ylmethyl)amino]-1- piperidinyl}methyl)-3-fluoro- 5,6-dihydro-8H- [1,4]oxazino[2,3,4-de]-1,5- naphthyridin-8-one hydrochloride | Intermediate 2 | 451 MH+, 30% | 1.31-1.54 (2H, m), 1.83-2.61 (9H, m), 2.78-2.89 (1H, m), 2.95 (2H, t, J = 6 Hz), 3.07-3.22 (3H, m), 4.04 (2H, s), 4.07- 4.15 (1H, m), 4.88-4.97 (1H, m), 5.07 (1H, d, J = 11 Hz), 6.82 (1H, d, J = 10 Hz), 7.35 (1H, s), 7.85 (1H, d, J = 10 Hz), 8.38 (1H, d, J = 2 Hz), |
| 88 | 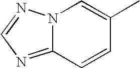 | (6R)-3-fluoro-6-({4- [([1,2,4]triazolo[1,5-a]pyridin- 6-ylmethyl)amino]-1- piperidinyl}methyl)-5,6- dihydro-8H- [1,4]oxazino[2,3,4-de]-1,5- naphthyridin-8-one | Intermediate 4 | 450 MH+, 40% | |

TABLE 5

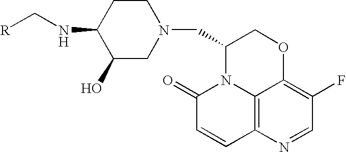

| Exam- | | | | MS ES+ | Nmr of free base |
|---|---|---|---|---|---|
| ple | Structure | Name | Aldehyde | m/z | (δ (ppm), CDCl3, 400 MHz) |
| 50 | 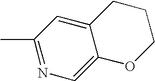 | (6R)-6-({(3R,4S)-4-[(3,4- dihydro-2H-pyrano[2,3- c]pyridin-6-ylmethyl)amino]- 3-hydroxy-1- piperidinyl}methyl)-3-fluoro- 5,6-sihydro-8H- [1,4]oxazino[2,3,4-de]-1,5- naphthyridin-8-one hydrochloride | WO2004058144 Example 126(e) | 482 MH+, 40% | 1.59-1.82 (2H, s), 1.97-2.07 (2H, m), 2.29-2.68 (6H, m), 2.71-2.87 (3H, s), 3.18-3.28 (1H, m), 3.82 (2H, s), 4.07- 4.25 (3H, m), 4.89-5.01 (1H, m), 5.05 (1H, d, J = 11 Hz), 6.82(1H, d, J = 10 Hz), 6.95 (1H, s), 7.84 (1H, d, J = 10 Hz), 8.05 ]1H, s), 8.38 (1H, d, J = 2 Hz). |
| 51 | 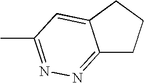 | (6R)-6-({(3R,4S)-4-[(6,7- dihydro-5H- cyclopenta[c]pyridazin-3- ylmethyl)amino]-3-hydroxy-1- piperidinyl}methyl)-3-fluoro- 5,6-dihydro-8H- [1,4]oxazino[2,3,4-de]-1,5- naphthyridin-8-one hydrochloride | Intermediate 2 | 467 MH+, 25% | 1.65-1.78 (2H, m), 1.95-2.68 (7H, m), 2.78-2.89 (1H, m), 2.98 (2H, t, J = 6 Hz), 3.12-3.24 (3H, m), 3.81-3.88 (1H, m), 4.09 (2H, s), 4.11-4.19 (1H, m), 4.92- 5.01 (1H, m), 5.03 (1H, d, J =11 Hz), 6.84 (1H, d, J10 Hz), 7.37 (1H, s), 7.85 (1H, d, J = 10 Hz), 8.37 (1H, d, J = 2 Hz). |
| 52 | 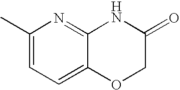 | (6R)-3-fluoro-6-[((3R,4S)-3- hydroxy-4-{[(3-oxo-3,4- dihydro-2H-pyrido[3,2- b][1,4]oxazin-6- yl)methyl]amino}-1- piperidinyl)methyl]-5,6- dihydro-8H- [1,4]oxazino[2,3,4-de]-1,5- naphthyridin-8-one hydrochloride | WO2003 087098 Example 31(e) | 497 MH+, 100% | 1.65-1.88 (2H, m), 2.28-2.71 (6H, m), 2.79-2.89 (1H, m), 3.18-3.26 (1H, m), 3.81-3.91 (3H, m), 4.11-4.19 (1H, m), 4.62 (2H, s), 4.91-5.01 (1H, m), 5.05 (1H,d, J = 11 Hz), 6.83 (1H, d, J = 10 Hz), 6.92 (1H, d, J = 8 Hz), 7.19 (1H, d, J = 8 Hz), 7.88 (1H, d, J = 10 Hz), 8.39 (1H, d, J = 2 Hz). |
| 53 | 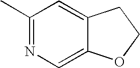 | (6R)-6-({(3R,4S)-4-[(2,3- dihydrofuro[2,3-c]pyridin-5- ylmethyl)amino]-3-hydroxy-1- piperidinyl}methyl)-3-fluoro- 5,6-dihydro-8H- [1,4]oxazino[2,3,4-de]-1,5- naphthyridin-8-one hydrochloride | Intermediate 5 | 468 MH+, 100% | 1.62-1.89 (2H, m), 2.15-2.66 (8H, m), 2.77-2.87 (1H, m), 3.15-3.29 (3H, m, (inc 3.23 (2H, t, J = 8.7 Hz))), 3.79-?? 3.89 (2H, m), 4.11-4.17 (1H, m), 4.61 (2H, t, J = 8.7 Hz), 4.89-5.01 (1H,m), 5.05 (1H, d, J = 11.2 Hz), 6.81 (1H, d, J = 9.7 Hz), 7.19 (1H, s), 7.83 (1H, d, J = 9.7 Hz), 8.07 (1H, s), 8.38 (1H, d, J2 = Hz). |
| 54 | 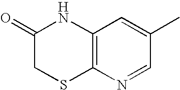 | (6R)-3-fluoro-6-[((3R,4S)-3- hydroxy-4-{[(2-oxo-2,3- dihydro-1H-pyrido[2,3- b][1,4]thiazin-7- yl)methyl]amino}-1- piperidinyl)methyl]-5,6- dihydro-8H- [1,4]oxazino[2,3,4-de]-1,5- naphthyridin-8-one hydrochloride | WO2004058144 Example 48 | 513 MH+, 100% | 1H NMR (250 MHz, DMSO-d6). δ 1.39-1.52 (1H, m), 1.55-1.71 (1H, m), 1.79-1.92 (1H, m), 2.15-2.69 (7H, m), 2.71-2.89 (1H, s), 3.54-3.78 (4H, m), 4.21-4.49 (2H,m), 4.81-4.99 (2H, m), 6.83 (1H, d, J = 9.7 Hz), 7.27 (1H, m), 7.93 (1H, d, J = 9.7 Hz), 8.04 (1H, s), 8.51 (1H, s), 10.62 (1H, s). |
| 97 | 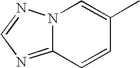 | (6R)-3-fluoro-6-({(3R,4S)-3- hydroxy-4- [([1,2,4]triazolo[1,5-a]pyridin- 6-ylmethyl)amino]-1- piperidinyl}methyl)-5,6- dihydro-8H- [1,4]oxazino[2,3,4-de]-1,5- naphthyridin-8-one | Intermediate 4 | 466 MH+, 25% | |

TABLE 6

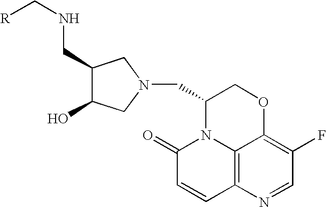

| Exam- | | | | MS ES+ | Nmr of free base |
|---|---|---|---|---|---|
| ple | R | Name | Aldehyde | m/z | (δ (ppm), CDCl3, 400 MHz) |
| 55 | 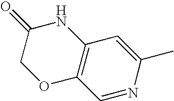 | (6R)-3-fluoro-6-{[(3R,4S)-3- hydroxy-4-({[(2-oxo-2,3- dihydro-1H-pyrido[3,4- b][1,4]oxazin-7- yl)methyl]amino}methyl)-1- pyrrolidinyl]methyl}-5,6- dihydro-8H- [1,4]oxazino[2,3,4-de]-1,5- naphthyridin-8-one hydrochloride | WO200387098 Example 309(i) | 497 MH+, 80% | 2.36-3.05 (10H, m), 3.89 (2H, s), 4.09- 4.19 (1H, m), 4.39-4.47 (1H, m), 4.63 (2H, s), 4.99 (1H, d, J = 11 Hz), 4.97-5.05 (1H, m), 6.88 (1H, d,J = 10 Hz), 6.95 (1H, s), 7.88 (1H, d, J = 10 Hz), 8.15 (1H, s), 8.39 (1H, d, J = 2 Hz). |
| 56 | 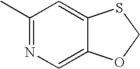 | (6R)-3-fluoro-6-[((3R,4S)-3- hydroxy-4- {[([1,3]oxathiolo[5,4- c]pyridin-6- ylmethyl)amino]methyl}-1- pyrrolidinyl)methyl]-5,6- dihydro-8H- [1,4]oxazino[2,3,4-de]-1,5- naphthyridin-8-one hydrochloride | WO2004058144 Example 61 | 486 MH+, 100% | 2.35-3.35 (10H, m), 3.81 (2H, s), 4.08- 4.15 (1H, m), 4.41-4.49 (1H, m), 4.95- 5.05 (1H, m), 5.08 (1H, d, J = 11 Hz), 5.73 (2H, s), 6.78 (1H, d, J = 10 Hz), 7.12 (1H, s), 7.84 (1H, d, J = 10 Hz), 8.03 (1H, s), 8.38 (1H, d, J = 2 Hz). |
| 57 | 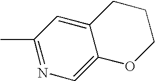 | (6R)-6-[((3R,4S)-3-{[(3,4- dihydro-2H-pyrano[2,3- c]pyridin-6- ylmethyl)amino]methyl}-4- hydroxy-1- pyrrolidinyl)methyl]-3-fluoro- 5,6-dihydro-8H- [1,4]oxazino[2,3,4-de]-1,5- naphthyridin-8-one hydrochloride | WO2004 058144 Example 126(e) | 482 MH+, 100% | 1.93-2.11 (2H, m), 2.33-2.39 (13H, m), 3.78 (2H, s), 4.07-4.14 (1H, m), 4.16- 4.22 (2H, m), 4.42-4.51 (1H, m), 4.93- 5.02 (1H, m), 5.06 (1H, d, J = 11 Hz), 6.79 (1H, d, J = 10 Hz), 6.91 (1H, s), 7.83 (1H, d, J = 10 Hz), 8.11 (1H, s), 8.39 (1H, d, J = 2 Hz). |
| 58 | 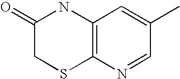 | (6R)-3-fluoro-6-{[(3S,4S)-3- hydroxy-4-({[(2-oxo-2,3- dihydro-1H-pyrido[2,3- b][1,4]thiazin-7- yl)methyl]amino}methyl)-1- pyrrolidinyl]methyl}-5,6- dihydro-8H- [1,4]oxazino[2,3,4-de]-1,5- naphthyridin-8-one hydrochloride | WO2004 058144 Example 48 | 513 MH+, 100% | 2.35-2.49 (1H, m),2.61-2.95 (9H, m), 3.57 (2H, s), 3.77 (2H, s), 4.11-4.18 (1H, m), 4.37-4.42 (1H, m), 4.92 (1H, d, J = 11 Hz), 4.99-5.08 (1H,m), 6.81 (1H, d, J = 10 Hz), 7.26 (1H, s), 7.83 (1H, d, J = 10 Hz), 8.1 (1H, s), 8.39 (1H, d, J = 2 Hz). |
| 89 | 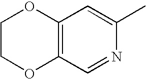 | (6R)-6-[((3R,4S)-3-{[(2,3- dihydro[1,4]dioxino[2,3- c]pyridin-7- ylmethyl)amino]methyl}-4- hydroxy-1- pyrrolidinyl)methyl]-3-fluoro- 5,6-dihydro-8H- [1,4]oxazino[2,3,4-de]-1,5- naphthyridin-8-one hydrochloride | WO2004058144, Example 2(c) or WO2003087098 Example 19(d) | 484 MH+, 100% | |

TABLE 7

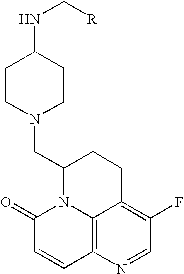

| Exam- | | | | MS ES+ | Nmr of free base |
|---|---|---|---|---|---|
| ple | R | Name | Aldehyde | m/z | (δ (ppm), CDCl3, 400 MHz) |
| 59 | 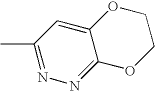 | 6-({4-[(6,7- dihydro[1,4]dioxino[2,3- c]pyridazin-3- ylmethyl)amino]-1- piperidinyl}methyl)-3-fluoro- 5,6-dihydro-4H,8H- pyrido[3,2,1-de]-1,5- naphthyridin-8-one hydrochloride | Example 3(e) | 467 MH+, 40%, 489 MNa+, 10% | 1.36-1.48 (2H, m), 1.71-1.80 (1H, m), 1.88-1.96 (3H, m), 2.08-2.12 (1H, m), 2.20-2.23 (1H, m), 2.32-2.36 (1H, m), 2.48-2.53 (2H, m), 2.62-2.86 (3H, m), 3.03-3.12 (2H, m), 4.00 (2H, s), 4.38- 4.41(2H, m), 4.50-4.52 (2H, m), 5.21- 5.24 (1H, m), 6.81 (1H, d, J = 10.0 Hz), 7.00 (1H, s), 7.86 (1H, d, J = 10.0 Hz), 8.35 (1H, s) |
| 60 | 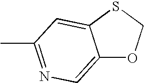 | 3-fluoro-6-({4- [([1,3]oxathiolo[5,4-c]pyridin- 6-ylmethyl)amino]-1- piperidinyl}methyl)-5,6- dihydro-4H,8H-pyrido[3,2,1- de]-1,5-naphthyridin-8-one hydrochloride | WO2004058144 Example 61 | 468 MH+, 40% | 1.36-1.48 (2H, m), 1.71-1.92 (4H, m), 2.08-2.12 (1H, m), 2.20-2.23 (1H, m), 2.32-2.36 (1H, m), 2.48-2.53 (2H, m), 2.62-2.85 (3H, m), 3.00-3.10 (2H, m), 3.80 (2H, s), 5.21-5.24 (1H, m), 5.73 (2H, s), 6.81 (1H, d, J = 10.0Hz), 7.20 (1H, s), 7.86 (1H, d, J = 10.0 Hz), 8.00 (1H, s), 8.35 (1H, s) |
| 61 | 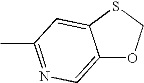 | 3-fluoro-6-({4- [([1,3]oxathiolo[5,4-c]pyridin- 6-ylmethyl)amino]-1- piperidinyl}methyl)-5,6- dihydro-4H,8H-pyrido[3,2,1- de]-1,5-naphthyridin-8-one | WO2004058144 Example 61 | 468 MH+, 40% | |
| 62 | 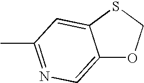 | 3-fluoro-6-({4- [([1,3]oxathiolo[5,4-c]pyridin- 6-ylmethyl)amino]-1- piperidinyl}methyl)-5,6- dihydro-4H,8H-pyrido[3,2,1- de]-1,5-naphthyridin-8-one Enantiomer 1 benzoate salt | prepared by chiral separation of racemic material (for a preparation see Example 61) | | Enantiomer 1 |
| 63 | 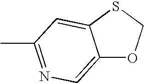 | 3-fluoro-6-({4- [([1,3]oxathiolo[5,4-c]pyridin- 6-ylmethyl)amino]-1- piperidinyl}methyl)-5,6- dihydro-4H,8H-pyrido[3,2,1- de]-1,5-naphthyridin-8-one Enantiomer 2 benzoate salt | prepared by chiral separation of racemic meterial (for a preparation see Example 61) | | Enantiomer 2 |
| 64 | 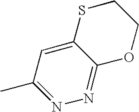 | 6-({4-[(6,7- dihydro[1,4]oxathiino[2,3- c]pyridazin-3- ylmethyl)amino]-1- piperidinyl}methyl)-3-fluoro- 5,6-dihydro-4H,8H- pyrido[3,2,1-de]-1,5- naphthyridin-8-one | Example 4(d) | 483 MH+, 40%, 505 MNa+ 10% | 1.51-1.85 (3H, m), 2.00-2.06 (2H, m), 2.18-2.25 (1H, m), 2.30-2.36 (1H, m), 2.48-2.53 (2H, m), 2.62-2.68 (2H, m), 2.82-2.92 (3H, m), 3.04-3.10 (1H, m), 3.21-3.25 (2H, m), 4.15 (2H, s), 4.68- 4.70 (2H, m), 5.21-5.24 (1H, m), 6.81 (1H, d, J = 10.0 Hz), 7.40 (1H, s), 7.86 (1H, d, J = 10.0 Hz), 8.35 (1H, s) |
| 65 | 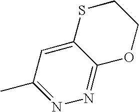 | 6-({4-[(6,7- dihydro[1,4]oxathiino[2,3- c]pyridazin-3- ylmethyl)amino]-1- piperidinyl}methyl)-3-fluoro- 5,6-dihydro-4H,8H- naphthyridin-8-one Enantiomer 1 | prepared by chiral separation of racemic material (for a preparartion see Example 64) | | Enantiomer 1 |
| 66 | 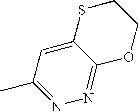 | 6-({4-[(6,7- dihydro[1,4]oxathiino[2,3- c]pyridazin-3- ylmethyl)amino]-1- piperidinyl}methyl)-3-fluoro- 5,6-dihydro-4H,8H- pyrido[3,2,1-de]-1,5- naphthyridin-8-one Enantiomer 2 | prepared by chiral separation of racemic material (for a preparation see Example 64) | | Enantiomer 2 |

TABLE 8

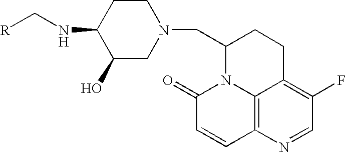

| Exam- | | | | MS ES+ | Nmr of free base |
|---|---|---|---|---|---|
| ple | R | Name | Aldehyde | m/z | (δ (ppm), CDCl3, 400 MHz) |
| 67 | 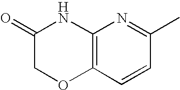 | 3-fluoro-6-[((3R,4S)-3- hydroxy-4-{[(3-oxo-3,4- dihydro-2H-pyrido[3,2- b][1,4]oxazin-6- yl)methyl]amino}-1- piperidinyl)methyl]-5,6- dihydro-4H,8H-pyrido[3,2,1- de]-1,5-naphthyridin-8-one hydrochloride | WO2003087098 Example 31(e) | 495 MH+, 100% | 1.55-2.95 (12H, m), 2.99-3.21 (2H, m), 3.78-3.95 (3H, m), 4.62 (2H, s), 5.19- 5.38 (1H, m), 6.82 (1H, dd, J = 4 and 10 Hz), 6.93 (1H, dd, J = 5 and 8 Hz), 7.18 (1H,d, J = 10 Hz), 7.88 (1H, d, J = 10 Hz), 8.38 (1H, d, J = 4 Hz). |
| 68 | 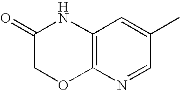 | 3-fluoro-6-[((3R,4S)-3- hydroxy-4-{[(2-oxo-2,3- dihydro-1H-pyrido[2,3- b][1,4]oxazin-7- yl)methyl]amino}-1- piperidinyl)methyl]-5,6- dihydro-4H,8H-pyrido[3,2,1- de]-1,5-naphthyridin-8-one hydrochloride | WO2003087098 Example 155(e) | 495 MH+, 80% | 1.45-2.88 (12H, m), 2.99-3.21 (2H, m), 3.68-3.95 (3H, m), 4.81 (2H, s), 5.19- 5.41 (1H, m), 6.83 (1H, dd, J = 4 and 10 Hz), 7.31 (1H, dd, J = 2 and 7 Hz), 7.77-7.91(2H, m), 8.38 (1H, d, J = 4 Hz). |

### Intermediate 1

### 5,6,7,8-tetrahydro-3-cinnolinecarbaldehyde

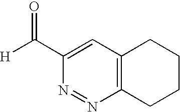

### (a) 3-Ethenyl-5,6,7,8-tetrahydrocinnoline

A solution of 3-chloro-5,6,7,8-tetrahydrocinnoline (for a synthesis see Arch. Pharm. 1989, 322(6), 331-6) (0.78 g, 4.6 mmol) and vinyl boronic anhydride.pyridine complex (0.442 g, 1.04 mmol) in a mixture of 1,4-dioxane (25 ml) and sodium bicarbonate (1.0 g in 4.8 ml water) was degassed by evacuation and purging with Argon. The degassing procedure was repeated three times, then the mixture treated with tetrakis(triphenylphosphine) palladium (0) and heated to reflux overnight. The solvent was evaporated and the residue partitioned between water (50 ml) and ethyl acetate (50 ml). The organic phase was washed with brine dried (MgSO4) and evaporated. The residue was chromatographed on silica gel eluring with 1-10% methanol in dichloromethane to give the title compound as a yellow oil. (0.48 g, 66%).

MS (ES+) m/z 161 (MH+, 100%).

### (b) Title Compound

3-Ethenyl-5,6,7,8-tetrahydrocinnoline (0.230 g, 1.4 mmol) was dissolved in a mixture of 1,4-dioxane (15 ml) and water (3.75 ml), cooled in an ice-bath, and treated with a solution of osmium tetroxide (4% aqueous solution, 1.5 ml) and sodium periodate (0.81 g). The reaction was stirred at 0° C. for 1 h, then evaporated under reduced pressure. The residue was partitioned between ethyl acetate and water. The organic phase was washed with water and brine, dried (MgSO4) and evaporated. The residue was chromatographed on silica eluting with 50-100% ethyl acetate in hexane to afford the title compound as a yellow solid (13 mg, 5.6%).

MS (ES+) m/z 163 (MH+, 100%).

### Intermediate 2

### 6,7-dihydro-5H-cyclopenta[c]pyridazine-3-carbaldehyde

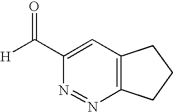

### (a) 3-Ethenyl-6,7-dihydro-5H-cyclopenta[c]pyridazine

3-Chloro-6,7-dihydro-5H-cyclopenta[c]pyridazine (2.0 g 13 mmol) (for a synthesis see Horning, R. H.; Amstutz, E. D. Journal of Organic Chemistry (1955), 20 707-13) was converted into the title compound using the general conditions given for Intermediate 1(a). The product was obtained as an off-white solid (4.45 g, 78%).

MS (ES+) m/z 147 (MH+, 100%).

### (b) Title Compound

3-Ethenyl-6,7-dihydro-5H-cyclopenta[c]pyridazine was converted into the title compound using the general conditions of Intermediate 1(b), but increasing the reaction time to 18 h at room temperature. After work-up and chromatography, the product was obtained as a an off-white solid (0.277 g, 18%).

MS (ES+) m/z 149 (MH+, 100%), 181 (MeOH adduct, 20%).

### Intermediate 3

### 7,8-dihydro-5H-pyrano[4,3-c]pyridazine-3-carbaldehyde

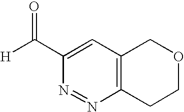

### (a) 7,8-Dihydro-2H-pyrano[4,3-c]pyridazin-3(5H)-one

Tetrahydro-4H-pyran-4-one (4.12 g, 41 mmol) was heated with glyoxylic acid monohydrate (3.8 g, 41 mmol) at 50° C. under an argon atmosphere. After 3 h, the reaction mixture was cooled in an ice-water bath and diluted with ethanol (25 ml). Hydrazine hydrate (2.95 ml) was added dropwise over 10 min, and the reaction then heated to reflux for 6 h. The reaction mixture was cooled to room temperature and left to stand overnight. The resultant yellow crystals were collected by filtration and dried in vacuo, to give the title compound, 1.54 g, (24%).

MS (ES+) m/z 153 (MH+, 100%), 175 (MNa+, 20%).

### (b) 3-Chloro-7,8-dihydro-5H-pyrano[4,3-c]pyridazine

7,8-Dihydro-2H-pyrano[4,3-c]pyridazin-3(5H)-one (1.65 g, 10.7 mmol) was suspended in phosphoryl chloride and the mixture heated to 100° C. for 1 h, then cooled and evaporated. The residue was dissolved in dichloromethane (120 ml) and treated cautiously with saturated sodium bicarbonate solution (60 ml). The mixture was stirred for 1 h until gas evolution had ceased, then the phases separated. The organic phase was dried (MgSO4) and evaporated to give the title compound as a red-brown solid (1.34 g).

MS (ES+) m/z 171 (MH+, 100%).

### (c) 3-Ethenyl-7,8-dihydro-5H-pyrano[4,3-c]pyridazine

3-Chloro-7,8-dihydro-5H-pyrano[4,3-c]pyridazine (1.33 g, 7.8 mmol) was converted into the title compound using the general procedure of Intermediate 1(a). After chromatography, the title compound was obtained as a pale yellow solid (1.09 g, 86%).

MS (ES+) m/z 163 (MH+, 100%).

### (d) Title Compound

3-Ethenyl-7,8-dihydro-5H-pyrano[4,3-c]pyridazine (0.163 g, 1.0 mmol) was converted to the title compound under the general conditions of Intermediate 1(b), but increasing the reaction time to 18 h at room temperature. After work-up using dichloromethane in place of ethyl acetate followed by chromatography, the product was obtained as a white solid (0.043 g, 26%).

MS (ES+) m/z 165 (MH+, 100%), 197 (MH+, methanol adduct, 20%).

### Intermediate 4

### [1,2,4]triazolo[1,5-a]pyridine-6-carbaldehyde

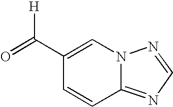

### (a) 6-Bromo[1,2,4]triazolo[1,5-a]pyridine

To a stirred solution of 2-amino-5-bromo-pyridine (5 g, 28.9 mmol) in DMF (12 mL) was added DMF-DMA (12 mL) and the solution was heated at 130° C. overnight. The reaction was then cooled to room temperature and the volatiles removed to afford the desired intermediate N-(5-bromo-2-pyridinyl)imidoformamide.

¹H NMR (400 MHz) δ(DMSO) 6.76 (1H, d), 7.73 (1H), 8.24 (1H, m), 8.45 (1H, s)

The intermediate was dissolved in methanol (40 mL), treated with pyridine (4.6 mL) and cooled to 0° C. Hydroxylamine-o-sulfonic acid (4.52 g) was added and the reaction mixture was allowed to warm to room temperature and stirred at room temperature overnight. The solvents were then removed and the residue was partitioned between a saturated solution of sodium bicarbonate and 20% methanol in dichloromethane. The phases were separated and the aqueous layer was extracted again with 20% methanol in dichloromethane (2×250 mL). The organics were washed with water, brine, dried (magnesium sulphate), filtered and reduced to afford 4.7 g of crude product. This was dissolved in 20% methanol in dichloromethane and washed with brine, then dried, filtered and evaporated to afford 4 g of impure product. The residue was subjected to column chromatography on silica gel to afford 2.37 g of the title compound.

MS (ES+) m/z 198/200 (MH⁺)

### (b) 6-Ethenyl[1,2,4]triazolo[1,5-c]pyridine

6-Bromo[1,2,4]triazolo[1,5-a]pyridine (0.5 g, 2.5 mmol) was dissolved in degassed DME (20 mL) under argon and tetrakis triphenylphosphine palladium (58 mg, 0.05 mmol) was added under argon and the orange solution was stirred at room temperature under argon for about 40 minutes. Potassium carbonate (345 mg, 2.5 mmol), water (4 mL) and triethenylboroxin pyridine complex (for a synthesis see Kerins, F.; O'Shea, D. J. Org. Chem. (2002), 67(14), 4968-4971) (162 mg, 1 mmol) were then added and the mixture was stirred at reflux for 4 h (the colour of the reaction changed to yellow). The reaction was stirred at reflux overnight. A further 1% of tetrakis triphenylphosphine palladium, 0.2 eq of triethenylboroxin pyridine complex and 100 mg of potassium carbonate were added and the reaction was stirred at reflux for 1 h. The reaction was cooled to room temperature, diethylether was added and the aqueous was extracted (3×80 mL). The organics were dried (magnesium sulphate), filtered and reduced to afford a solid which was subjected to silica chromatography (0-5% methanol in dichloromethane) to afford the title compound (340 mg, 93%).

MS (ES+) m/z 146 (MH⁺).

### (c) Title Compound

A solution of 6-ethenyl[1,2,4]triazolo[1,5-a]pyridine (340 mg, 2.34 mmol) in 1,4-dioxane/water (10/5 mL) was treated with sodium periodate (1.25 g, 5.85 mmol) and 4% osmium tetroxide solution in water (0.45 mL) at room temperature for 30 minutes.

The dioxane was evaporated and the aqueous was extracted with 20% methanol in dichloromethane (2×100 mL). The combined organics were dried on magnesium sulphate, filtered and evaporated to give 330 mg of the desired product.

MS (ES+) m/z 148 (MH+)

¹H NMR (250 MHz) δ(CDCl3) 10.1 (1H, s), 9.12 (1H, m), 8.51 (1H, s), 8.04 (1H, d), 7.79 (1H, d).

### Intermediate 5

### 2,3-Dihydrofuro[2,3-c]pyridine-5-carbaldehyde

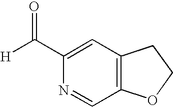

### (a) {5-({[4-(Methyloxy)phenyl]methyl}oxy)-4-[(trimethylsilyl)ethynyl]-2-pyridinyl}methyl acetate

(5-({[4-(Methoxy)phenyl]methyl}oxy)-4-{[(trifluoromethyl)sulfonyl]oxy}-2-pyridinyl)methyl acetate (for a synthesis, see WO2004058144 Example 60(d)) (10 g, 23 mmoles) was dissolved in acetonitrile (400 ml) and triethylamine (65 ml) and copper (I) iodide (0.44 g, 2.3 mmoles) were added. The mixture was degassed and placed under a blanket of argon. Trimethylsilylacetylene (10 ml, 69 mmoles) and bis(triphenylphosphine)palladium(II) dichloride (0.645 g, 0.9 mmoles) were added and the mixture heated to 45° C. for 18 hrs. The mixture was then allowed to cool and filtered. The filtrate was evaporated to dryness and the residue partitioned between ethyl acetate and water. The organic layer was separated and dried (sodium sulphate).

Chromatography on silica gel, eluting with a gradient of 20-75% ethyl acetate in 40-60 petroleum ether, gave an oil (8.45 g, 96%).

MS (+ve ion electrospray) m/z 384 (MH+).

### (b) {5-Hydroxy-4-[(trimethylsilyl)ethynyl]-2-pyridinyl}methyl acetate, trifluoroacetate

{5-({[4-(Methyloxy)phenyl]methyl}oxy)-4-[(trimethylsilyl)ethynyl]-2-pyridinyl}methyl acetate (8.45 g, 22 mmoles) in dichloromethane (70 ml) was treated with trifluoroacetic acid (9.4 ml) and triethylsilane (3.33 ml) and stirred at ambient temperature for 18 hrs. The mixture was evaporated to dryness and chromatographed on silica gel, eluting with a gradient of 2-8% methanol in dichloromethane. This gave an oil (10 g, 100%).

MS (+ve ion electrospray) m/z 264 (MH+).

### (c) Furo[2,3-c]pyridin-5-ylmethyl acetate

{5-Hydroxy-4-[(trimethylsilyl)ethynyl]-2-pyridinyl}methyl acetate, trifluoroacetate) (10 g, 22 mmoles) was dissolved in pyridine (200 ml) and treated with copper(I) iodide (5.2 g, 27 mmoles) then heated under reflux for 18 hrs. The mixture was allowed to cool, evaporated to dryness and the residue partitioned between ethyl acetate and water. This mixture was filtered through kieselguhr to remove copper residues. The organic layer was separated from the filtrate, dried and chromatographed on silica gel, eluting with a gradient of 10-60% ethyl acetate in 40-60 petroleum ether. This gave furo[2,3-c]pyridin-5-ylmethyl acetate (1.15 g, 27%) and a less polar product [2-(trimethylsilyl)furo[2,3-c]pyridin-5-yl]methyl acetate (1.3 g, 23%) as oils.

MS (+ve ion electrospray) m/z 192 (MH+) and MS (+ve ion electrospray) m/z 264 (MH+).

### (d) Furo[2,3-c]pyridin-5-ylmethanol

A solution of furo[2,3-c]pyridin-5-ylmethyl acetate (1.15 g) in 1,4-dioxane (30 ml) and water (10 ml) was treated with 2M sodium hydroxide (12 ml) then stirred at ambient temperature for 18 hrs. The mixture was then partitioned between ethyl acetate and water. The organics were separated and dried then evaporated to dryness. This gave an oil (0.63 g, 70%).

MS (+ve ion electrospray) m/z 150 (MH+).

### (e) 2,3-Dihydrofuro[2,3-c]pyridin-5-ylmethanol

Furo[2,3-c]pyridin-5-ylmethanol (1.29 g, 8.7 mmoles) was dissolved in ethanol (50 ml) and hydrogenated at S.T.P (standard temperature and pressure) over 10% palladium on charcoal paste for 18 hrs. The mixture was filtered through kieselguhr and the filtrate evaporated to dryness, to give (1.31 g, 100%).

MS (+ve ion electrospray) m/z 152 (MH+).

### (f) 2,3-Dihydrofuro[2,3-c]pyridine-5-carbaldehyde

2,3-Dihydrofuro[2,3-c]pyridin-5-ylmethanol (1.31 g, 8.7 mmoles) was dissolved in dichloromethane (100 ml), treated with manganese (IV) dioxide (6 g, 69 mmoles) and heated under reflux for 18 hrs. Filtration through kieselguhr and evaporation of the filtrate to dryness gave an oil (0.9 g, 70%).

MS (+ve ion electrospray) m/z 150 (MH+).

1. A compound of formula (I) or a pharmaceutically acceptable salt and/or N-oxide thereof: 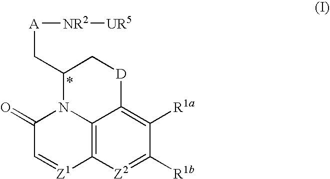
wherein:
D is O, S or CH₂;
one of Z¹ and Z² is N or CR^{1c} and the other is independently CR^{1c};
R^{1a}, R^{1b} and R^{1c} are independenly selected from hydrogen; halogen; cyano; (C₁₋₆)alkyl; (C₁₋₆)alkylthio; trifluoromethyl; trifluoromethoxy; carboxy; hydroxy optionally substituted with (C₁₋₆)alkyl or (C₁₋₆)alkoxy-substituted(C₁₋₆)alkyl; (C₁₋₆)alkoxy-substituted(C₁₋₆)alkyl; hydroxy (C₁₋₆)alkyl; an amino group optionally N-substituted by one or two (C₁₋₆)alkyl, formyl, (C₁₋₆)alkylcarbonyl or (C₁₋₆)alkylsulphonyl groups; and aminocarbonyl wherein the amino group is optionally substituted by (C₁₋₄)alkyl;
or when one of Z¹ and Z² is CR^{1c}, R^{1c} may instead be:
(C₃₋₆)cycloalkyl; (C₃₋₆)cycloalkoxy; (C₂₋₆)alkenyl optionally substituted by carboxy, (C₁₋₆)alkoxycarbonyl or aminocarbonyl wherein the amino group is optionally substituted by one or two (C₁₋₄)alkyl; (C₁₋₆)alkylcarbonyl; (C₁₋₆)alkylcarbonyl oxime; (C₁₋₄)alkyloxycarbonyl(C₁₋₆)allyloxy; (C₁₋₄)alkylaminocarbonyl(C₁₋₆)alkyloxy; amino substituted by (C₁₋₄)alkylaminocarbonyl; aminocarbonyl wherein the amino group is substituted by (C₁₋₄)alkoxysulphonyl, hydroxy(C₁₋₄)alkyl, (C₁₋₄)alkoxy-substituted (C₁₋)alkyl, (C₃₋₆)cycloalkyl, phenyl, benzyl, monocyclic heteroaryl or monocyclic heteroaryl-methyl; benzyloxy; phenyl; benzyl; monocyclic heteroaryl; or monocyclic heteroaryl-methyl;
wherein heteroaryl is a 5 or 6 membered ring containing up to four hetero-atoms selected from oxygen, nitrogen and sulphur, and wherein a heteroaryl or phenyl ring in R^{1b} may be optionally C-substituted by up to three groups selected from (C₁₋₄)alkylthio; halo; carboxy(C₁₋₄)alkyl; halo(C₁₋₄)alkoxy; halo(C₁₋₄)alkyl; (C₁₋₄)allyl; (C₂₋₄)alkenyl; (C₁₋₄)alkoxycarbonyl; formyl; (C₁₋₄)alkylcarbonyl; (C₂₋₄)alkenyloxycarbonyl; (C₂₋₄)alkenylcarbonyl; (C₁₋₄)alkylcarbonyloxy; (C₁₋₄)alkoxycarbonyl(C₁₋₄)alkyl; hydroxy; hydroxy(C₁₋₄)alkyl; mercapto(C₁₋₄)alkyl; (C₁₋₄)alkoxy; nitro; cyano; carboxy; amino or aminocarbonyl optionally substituted by one or two (C₁₋₄)alkyl; (C₁₋₄)alkylsulphonyl; (C₂₋₄)alkenylsulphonyl; or aminosulphonyl wherein the amino group is optionally substituted by (C₁₋₄)allyl or (C₂₋₄)alkenyl;
R² is hydrogen, or (C₁₋₄)allyl, or together with R⁶ forms Y as defined below;
A is a group (i): 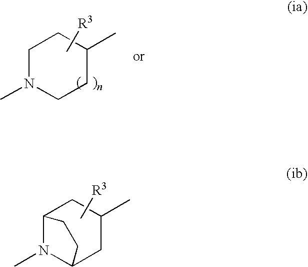
in which: R³ is as defined for R^{1a} or R^{1b}, provided that R³ in the 4-position is other than carboxy, or R³ is oxo and n is 1 or 2:
or A is a group (ii) 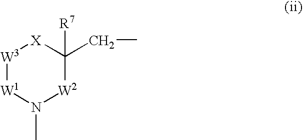
W¹, W² and W³ are CR⁴R⁸;
or W² and W³ are CR⁴R⁸ and W¹ represents a bond between W³ and N;
X is O, CR⁴R⁸, or NR⁶;
one R⁴ is as defined for R^{1a} and R^{1b} and the remainder and R⁸ are hydrogen or one R⁴ and R⁸ are together oxo and the remainder are hydrogen;
R⁶ is hydrogen or (C₁₋₆)alkyl; or together with R² forms Y;
R⁷ is hydrogen; halogen; hydroxy optionally substituted with (C₁₋₆)alkyl; or (C₁₋₆)alkyl;
Y is CR⁴R⁸CH₂; CH₂CR⁴R⁸; (C═O); CR⁴R⁸; CR⁴R⁸(C═O); or (C═O)CR⁴R⁸;
or when X is CR⁴R⁸, R⁸ and R⁷ together represent a bond;
U is selected from CO and CH₂ and
R⁵ is an optionally substituted bicyclic carbocyclic or heterocyclic ring system (B): 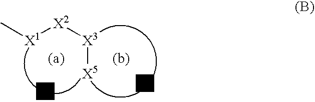
containing up to four heteroatoms in each ring in which
at least one of rings (a) and (b) is aromatic;
X¹ is C or N when part of an aromatic ring, or CR¹⁴ when part of a non-aromatic ring;
X² is N, NR¹³, O, S(O)_{X}, CO or CR¹⁴ when part of an aromatic or non-aromatic ring or may in addition be CR¹⁴R¹⁵ when part of a non aromatic ring;
X³ and X⁵ are independently N or C;
Y¹ is a 0 to 4 atom linker group each atom of which is independently selected from N, NR¹³, O, S(O)_{X}, CO and CR¹⁴ when part of an aromatic or non-aromatic ring or may additionally be CR¹⁴R¹⁵ when part of a non aromatic ring;
Y² is a 2 to 6 atom linker group, each atom of Y² being independently selected from N, NR¹³, O, S(O)_{X}, CO, CR¹⁴ when part of an aromatic or non-aromatic ring or may additionally be CR¹⁴R¹⁵ when part of a non aromatic ring;
each of R¹⁴ and R¹⁵ is independently selected from: H; (C₁₋₄)alkylthio; halo; carboxy(C₁₋₄)alkyl; (C₁₋₄)alkyl; (C₁₋₄)alkoxycarbonyl; (C₁₋₄)alkylcarbonyl; (C₁₋₄)alkoxy (C₁₋₄)alkyl; hydroxy; hydroxy(C₁₋₄)alkyl; (C₁₋₄)alkoxy; nitro; cyano; carboxy; amino or aminocarbonyl optionally mono- or di-substituted by (C₁₋₄)alkyl; or
R¹⁴ and R¹⁵ may together represent oxo;
each R¹³ is independently H; trifluoromethyl; (C₁₋₄)alkyl optionally substituted by hydroxy, (C₁₋₆)alkoxy, (C₁₋₆)alkylthio, halo or trifluoromethyl; (C₂₋₄)alkenyl; (C₁₋₄)alkoxycarbonyl; (C₁₋₄)alkylcarbonyl; (C₁₋₆)alkylsulphonyl; aminocarbonyl wherein the amino group is optionally mono or disubstituted by (C₁₋₄)alkyl; and
each x is independently 0, 1 or 2.

2. A compound according to claim 1 wherein:
Z¹ and Z² are both CH, R^{1a} is fluoro and R^{1b} is hydrogen;
Z¹ is CH and Z² is CR^{1c} where R^{1c} is cyano, —CH═CHCO₂H or —CH═CHCO₂C₂H₅, R^{1a} is fluoro and R^{1b} is hydrogen;
Z¹ is CH and Z² is N, R^{1a} is fluoro, bromo, cyano or methoxy and R^{1b} is hydrogen; or
Z¹ is N and Z² is CH and R^{1a} is chloro.

3. A compound according to claim 1 wherein A is a group (ia) in which n is 1 and R³ is hydrogen or hydroxy.

4. A compound according to claim 1 wherein A is (ii), X is CR⁴R⁸, R⁸ is H and R⁴ is H or OH, W¹ is a bond, W² and W³ are both CH₂ and R⁷ is H, or X is O, R⁷ is H and W¹, W² and W³ are each CH₂.

5. A compound according to claim 1 wherein R² is hydrogen.

6. A compound according to claim 1 wherein U is CH₂.

7. A compound according to claim 1 wherein R⁵ is an aromatic heterocyclic ring (B) having 8-11 ring atoms including 2-4 heteroatoms of which at least one is N or NR¹³ in which Y² contains 2-3 heteroatoms, one of which is S and 1-2 are N, with one N bonded to X³, or the heterocyclic ring (B) has ring (a) aromatic selected from optionally substituted benzo, pyrido, pyridazino and pyrimidino and ring (b) non aromatic and Y² has 3-5 atoms, more particularly 4 atoms, including at least one heteroatom, with O, S, CH₂ or NR¹³ bonded to X⁵ where R¹³ is other than hydrogen, and either NHCO bonded via N to X³, or O, S, CH₂ or NH bonded to X³.

8. A compound according to claim 1 wherein R⁵ is selected from the group consisting of:
3-oxo-3,4-dihydro-2H-pyrido[3,2-b][1,4]oxazin-6-yl;
3-oxo-3,4-dihydro-2H-pyrido[3,2-b][1,4]thiazin-6-yl;
6,7-dihydro[1,4]dioxino[2,3-c]pyridazin-3-yl;
6,7-dihydro[1,4]oxathiino[2,3-c]pyridazin-3-yl;
2,3-dihydro-[1,4]dioxino[2,3-c]pyridin-7-yl;
[1,3]oxathiolo[5,4-c]pyridin-6-yl;
3,4-dihydro-2H-pyrano[2,3-c]pyridin-6-yl;
2,3-dihydro[1,4]oxathiino[2,3-c]pyridin-7-yl.

9. A compound according to claim 1 selected from a group consisting of:
3-({4-[(2,3-Dihydro[1,4]dioxino[2,3-c]pyridin-7-ylmethyl)amino]-1-piperidinyl}methyl)-10-fluoro-2,3-dihydro-1H,5H-pyrido[3,2,1-ij]quinolin-5-one;
10-Fluoro-3-({4-[([1,3]oxathiolo[5,4-c]pyridin-6-ylmethyl)amino]-1-piperidinyl}methyl)-2,3-dihydro-1H,5H-pyrido[3,2,1-ij]quinolin-5-one;
3-({4-[(6,7-Dihydro[1,4]dioxino[2,3-c]pyridazin-3-ylmethyl)amino]-1-piperidinyl}methyl)-10-fluoro-2,3-dihydro-1H,5H-pyrido[3,2,1-ij]quinolin-5-one;
3-({4-[(6,7-Dihydro[1,4]oxathiino[2,3-c]pyridazin-3-ylmethyl)amino]-1-piperidinyl}methyl)-10-fluoro-2,3-dihydro-1H,5H-pyrido[3,2,1-ij]quinolin-5-one;
3-({4-[(6,7-Dihydro[1,4]dioxino[2,3-c]pyridazin-3-ylmethyl)amino]-1-piperidinyl}methyl)-10-fluoro-2,3-dihydro-1H,5H-pyrido[3,2,1-ij]quinolin-5-one
or an enantiomer thereof;
(6R)-3-Bromo-6-({4-[(2,3-dihydro[1,4]dioxino[2,3-c]pyridin-7-ylmethyl)amino]-1-piperidinyl}methyl)-5,6-dihydro-8H-[1,4]oxazino[2,3,4-de]-1,5-naphthyridin-8-one;
(6R)-6-({4-[(2,3-Dihydro[1,4]dioxino[2,3-c]pyridin-7-ylmethyl)amino]-1-piperidinyl}methyl)-3-fluoro-5,6-dihydro-8H-[1,4]oxazino[2,3,4-de]-1,5-naphthyridin-8-one;
(6R)-6-({4-[(2,3-Dihydro[1,4]dioxino[2,3-c]pyridin-7-ylmethyl)amino]-1-piperidinyl}methyl)-3-fluoro-5,6-dihydro-8H-[1,4]oxazino[2,3,4-de]-1,5-naphthyridin-8-one;
(6R)-6-({4-[(2,3-Dihydro[1,4]dioxin[2,3-c]pyridin-7-ylmethyl)amino]-1-piperidinyl}methyl)-3-fluoro-5,6-dihydro-8H-[1,4]oxazino[2,3,4-de]-1,5-naphthyridin-8-one;
(6R)-6-({4-[(2,3-Dihydro[1,4]dioxino[2,3-c]pyridin-7-ylmethyl)amino]-1-piperidinyl}methyl)-3-fluoro-5,6-dihydro-8H-[1,4]oxazino[2,3,4-de]-1,5-naphthyridin-8-one;
(3R)-3-({4-[(2,3-Dihydro[1,4]dioxino[2,3-c]pyridin-7-ylmethyl)amino]-1-piperidinyl}methyl)-10-fluoro-2,3-dihydro-5H-[1,4]oxazino[2,3,4-ij]quinolin-5-one;
Ethyl (2E)-3-[(3R)-3-({4-[(2,3-dihydro[1,4]dioxino[2,3-c]pyridin-7-ylmethyl)amino]-1-piperidinyl}methyl)-10-fluoro-5-oxo-2,3-dihydro-5H-[1,4]oxazino[2,3,4-ij]quinolin-8-yl]-2-propenoate;
(2E)-3-[(3R)-3-({4-[(2,3-Dihydro[1,4]dioxino[2,3-c]pyridin-7-ylmethyl) amino]-1-piperidinyl}methyl)-10-fluoro-5-oxo-2,3-dihydro-5H-[1,4]oxazino[2,3,4-ij]quinolin-8-yl]-2-propenoic acid;
6-({4-[(2,3-Dihydro[1,4]dioxino[2,3-c]pyridin-7-ylmethyl)amino]-1-piperidinyl}methyl)-3-fluoro-5,6-dihydro-4H,8H-pyrido[3,2,1-de]-1,5-naphthyridin-8-one;
6-({4-[(2,3-Dihydro[1,4]dioxino[2,3-c]pyridin-7-ylmethyl)amino]-1-piperidinyl}methyl)-3-fluoro-5,6-dihydro-4H,8H-pyrido[3,2,1-de]-1,5-naphthyridin-8-one Enantiomer 1;
6-({4-[(2,3-Dihydro[1,4]dioxino[2,3-c]pyridin-7-ylmethyl)amino]-1-piperidinyl}methyl)-3-fluoro-5,6-dihydro-4H,8H-pyrido[3,2,1-de]-1,5-naphthyridin-8-one Enantiomer 2;
6-({4-[(2,3-Dihydro[1,4]dioxino[2,3-c]pyridin-7-ylmethyl)amino]-1-piperidinyl}methyl)-3-fluoro-5,6-dihydro-4H,8H-pyrido[3,2,1-de]-1,5-naphthyridin-8-one Enantiomer 1;
(6R)-6-({4-[(2,3-Dihydro[1,4]dioxino[2,3-c]pyridin-7-ylmethyl)amino]-1-piperidinyl}methyl)-8-oxo-5,6-dihydro-8H-[1,4]oxazino[2,3,4-de]-1,5-naphthyridine-3-carbonitrile;
(3R)-3-({(3R,4S)-4-[(2,3-Dihydro[1,4]dioxino[2,3-c]pyridin-7-ylmethyl)amino]-3-hydroxy-1-piperidinyl}methyl)-10-fluoro-2,3-dihydro-5H-[1,4]oxazino[2,3,4-ij]quinolin-5-one;
(3R)-3-[((3S,4S)-3-{[(2,3-Dihydro[1,4]dioxino[2,3-c]pyridin-7-ylmethyl)amino]methyl}-4-hydroxy-1-pyrrolidinyl)methyl]-10-fluoro-2,3-dihydro-5H-[1,4]oxazino[2,3,4-ij]quinolin-5-one;
(6R)-6-({(3R,4S)-4-[(2,3-Dihydro[1,4]dioxino[2,3-c]pyridin-7-ylmethyl)amino]-3-hydroxy-1-piperidinyl}methyl)-3-fluoro-5,6-dihydro-8H-[1,4]oxazino[2,3,4-de]-1,5-naphthyridin-8-one;
(6R)-3-Fluoro-6-{[(3S,4S)-3-hydroxy-4-({[(3-oxo-3,4-dihydro-2H-pyrido[3,2-b][1,4]oxazin-6-yl)methyl]amino}methyl)-1-pyrrolidinyl]methyl}-5,6-dihydro-8H-[1,4]oxazino[2,3,4-de]-1,5-naphthyridin-8-one;
(6R)-6-({(3S,4R)-4-[(2,3-Dihydro[1,4]dioxino[2,3-c]pyridin-7-ylmethyl)amino]-3-hydroxy-1-piperidinyl}methyl)-3-fluoro-5,6-dihydro-8H-[1,4]oxazino[2,3,4-de]-1,5-naphthyridin-8-one;
(6R)-6-({(3S,4R)-4-[(3,4-Dihydro-2H-pyrano[2,3-c]pyridin-6-ylmethyl)amino]-3-hydroxy-1-piperidinyl}methyl)-3-fluoro-5,6-dihydro-8H-[1,4]oxazino[2,3,4-de]-1,5-naphthyridin-8-one;
(6R)-6-[(3-{[(2,3-Dihydro[1,4]dioxino[2,3-c]pyridin-7-ylmethyl)amino]methyl}-4-hydroxy-1-piperidinyl)methyl]-3-fluoro-5,6-dihydro-8H-[1,4]oxazino[2,3,4-de]-1,5-naphthyridin-8-one;
(6R)-3-Fluoro-6-[(4-hydroxy-3-{[([1,3]oxathiolo[5,4-c]pyridin-6-ylmethyl)amino]methyl}-1-piperidinyl)methyl]-5,6-dihydro-8H-[1,4]oxazino[2,3,4-de]-1,5-naphthyridin-8-one;
3-Fluoro-6-{[(3S,4S)-3-hydroxy-4-({[(3-oxo-3,4-dihydro-2H-pyrido[3,2-b][1,4]oxazin-6-yl)methyl]amino}methyl)-1-pyrrolidinyl]methyl}-5,6-dihydro-4H,8H-pyrido[3,2,1-de]-1,5-naphthyridin-8-one;
3-Fluoro-6-{[(3S,4S)-3-hydroxy-4-({[(2-oxo-2,3-dihydro-1H-pyrido[3,4-b][1,4]oxazin-7-yl)methyl]amino}methyl)-1-pyrrolidinyl]methyl}-5,6-dihydro-4H,8H-pyrido[3,2,1-de]-1,5-naphthyridin-8-one;
6-({(3R,4S)-4-[(2,3-dihydro[1,4]dioxino[2,3-c]pyridin-7-ylmethyl)amino]-3-hydroxy-1-piperidinyl}methyl)-3-fluoro-5,6-dihydro-4H,8H-pyrido[3,2,1-de]-1,5-naphthyridin-8-one;
(6R)-6-({4-[(2,3-dihydro[1,4]dioxino[2,3-c]pyridin-7-ylmethyl)amino]-1-piperidinyl}methyl)-3-fluoro-5,6-dihydro-8H-[1,4]thiazino[2,3,4-de]-1,5-naphthyridin-8-one;
(6R)-6-({4-[(6,7-dihydro[1,4]dioxino[2,3-c]pyridazin-3-ylmethyl)amino]-1-piperidinyl}methyl)-3-fluoro-5,6-dihydro-8H-[1,4]thiazino[2,3,4-de]-1,5-naphthyridin-8-one;
10-Chloro-3-({4-[(2,3-dihydro[1,4]dioxino[2,3-c]pyridin-7-ylmethyl)amino]-1-piperidinyl}methyl)-2,3-dihydro-5H-[1,4]oxazino[4,3,2-de]quinoxalin-5-one or an enantiomer thereof;
(6R)-6-({4-[(2,3-dihydro[1,4]dioxino[2,3-c]pyridin-7-ylmethyl)(methyl)amino]-1-piperidinyl}methyl)-3-fluoro-5,6-dihydro-8H-[1,4]oxazino[2,3,4-de]-1,5-naphthyridin-8-one;
(6S)-6-({4-[(2,3-dihydro[1,4]dioxino[2,3-c]pyridin-7-ylmethyl)amino]-1-piperidinyl}methyl)-3-fluoro-5,6-dihydro-8H-[1,4]oxazino[2,3,4-de]-1,5-naphthyridin-8-one;
(6S)-6-({4-[(6,7-dihydro[1,4]dioxino[2,3-c]pyridazin-3-ylmethyl)amino]-1-piperidinyl}methyl)-3-fluoro-5,6-dihydro-8H-[1,4]oxazino[2,3,4-de]-1,5-naphthyridin-8-one;
(6S)-3-fluoro-6-({4-[([1,3]oxathiolo[5,4-c]pyridin-6-ylmethyl)amino]-1-piperidinyl}methyl)-5,6-dihydro-8H-[1,4]oxazino[2,3,4-de]-1,5-naphthyridin-8-one;
(6R)-6-[(3-{[(2,3-dihydro[1,4]dioxino[2,3-c]pyridin-7-ylmethyl)amino]methyl}-4-hydroxy-1-piperidinyl)methyl]-3-fluoro-5,6-dihydro-8H-[1,4]oxazino[2,3,4-de]-1,5-naphthyridin-8-one;
(6R)-6-[(3-{[(3,4-dihydro-2H-pyrano[2,3-c]pyridin-6-ylmethyl)amino]methyl}-4-hydroxy-1-piperidinyl)methyl]-3-fluoro-5,6-dihydro-8H-[1,4]oxazino[2,3,4-de]-1,5-naphthyridin-8-one;
(6R)-6-{[4-[(2,3-Dihydro[1,4]dioxino[2,3-c]pyridin-7-ylmethyl)amino]-4-(hydroxymethyl)-1-piperidinyl]methyl}-3-fluoro-5,6-dihydro-8H-[1,4]oxazino[2,3,4-de]-1,5-naphthyridin-8-one;
N-(1-{[(6R)-3-Fluoro-8-oxo-5,6-dihydro-8H-[1,4]oxazino[2,3,4-de]-1,5-naphthyridin-6-yl]methyl}-4-piperidinyl)-3-oxo-3,4-dihydro-2H-pyrido[3,2-b][1,4]oxazine-6-carboxamide;
N-(1-{[(6R)-3-fluoro-8-oxo-5,6-dihydro-8H-[1,4]oxazino[2,3,4-de]-1,5-naphthyridin-6-yl]methyl}-4-piperidinyl)-3-oxo-3,4-dihydro-2H-pyrido[3,2-b][1,4]thiazine-6-carboxamide;
(6R)-6-[((3S)-3-{[(2,3-Dihydro[1,4]dioxino[2,3-c]pyridin-7-ylmethyl)amino]methyl}-1-pyrrolidinyl)methyl]-3-fluoro-5,6-dihydro-8H-[1,4]oxazino[2,3,4-de]-1,5-naphthyridin-8-one;
(6R)-6-[((3S,4R)-3-{[(3,4-dihydro-2H-pyrano[2,3-c]pyridin-6-ylmethyl)amino]methyl}-4-hydroxy-1-piperidinyl)methyl]-3-fluoro-5,6-dihydro-8H-[1,4]oxazino[2,3,4-de]-1,5-naphthyridin-8-one;
(6R)-6-[((3R)-3-{[(2,3-dihydro[1,4]dioxino[2,3-c]pyridin-7-ylmethyl)amino]methyl}-1-pyrrolidinyl)methyl]-3-fluoro-5,6-dihydro-8H-[1,4]oxazino[2,3,4-de]-1,5-naphthyridin-8-one;
(6R)-6-[((3R)-3-{[(6,7-dihydro[1,4]dioxino[2,3-c]pyridazin-3-ylmethyl)amino]methyl}-1-pyrrolidinyl)methyl]-3-fluoro-5,6-dihydro-8H-[1,4]oxazino[2,3,4-de]-1,5-naphthyridin-8-one;
6-({4-[(2,3-dihydro[1,4]dioxino[2,3-c]pyridin-7-ylmethyl)amino]-1-piperidinyl}methyl)-3-(methyloxy)-5,6-dihydro-4H,8H-pyrido[3,2,1-de]-1,5-naphthyridin-8-one;
3-(Methyloxy)-6-({4-[([1,3]oxathiolo[5,4-c]pyridin-6-ylmethyl)amino]-1-piperidinyl}methyl)-5,6-dihydro-4H,8H-pyrido[3,2,1-de]-1,5-naphthyridin-8-one;
6-({4-[(2,3-dihydro[1,4]dioxino[2,3-c]pyridin-7-ylmethyl)amino]-1-piperidinyl}methyl)-3-fluoro-5,6-dihydro-4H,8H-pyrido[3,2,1-de]-1,5-naphthyridin-8-one or an enantiomer thereof;
and
(6R)-6-[((3S,4S)-3-{[(2,3-dihydro[1,4]dioxino[2,3-c]pyridin-7-ylmethyl)amino]methyl}-4-hydroxy-1-pyrrolidinyl)methyl]-3-fluoro-5,6-dihydro-4H,8H-pyrido[3,2,1-de]-1,5-naphthyridin-8-one;
or a pharmaceutically acceptable salt thereof.

10. A method of treatment of bacterial infections in mammals, particularly in man, which method comprises the administration to a mammal in need of such treatment an effective amount of a compound according to claim 1.

11-13. (canceled)

14. A pharmaceutical composition comprising a compound according to claim 1 and a pharmaceutically acceptable carrier.
